(19) United States
(12) Patent Application Publication
(10) Pub. No.: US 2006/0229316 A1
(43) Pub. Date: Oct. 12, 2006

(54) Novel antiinfective compounds, process for their preparation and pharmaceutical compositions containing them

(76) Inventors: Braj B. Lohray, Gujarat (IN); Vidya B. Lohray, Gujarat (IN); Brijesh K. Srivastava, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 10/509,892

(22) PCT Filed: Mar. 26, 2003
(86) PCT No.: PCT/IN03/00081

§ 371 Date: May 17, 2005

(30) Foreign Application Priority Data
Apr. 1, 2002 (IN) 310MUM/2002

(51) Int. Cl. A61K 31/496; C07D 413/02

(52) U.S. Cl. 514/254.02; 544/369

(57) ABSTRACT

The present invention describes novel antiinfective compounds, process for their preparation and pharmaceutical compositions containing them.

### FIELD OF INVENTION

The present invention relates to novel compounds of general formula (I), their analogs, their derivatives, their stereoisomers, tautomeric forms, novel intermediates involved in their synthesis, their pharmaceutically acceptable salts and pharmaceutical compositions containing them. The present invention also relates to a process of preparing compounds of general formula (I), their analogs, their derivatives, their stereoisomers, their tautomeric forms, their pharmaceutically acceptable salts, pharmaceutically acceptable solvates, pharmaceutical compositions containing them, and novel intermediates 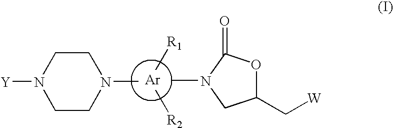
involved in their synthesis.

The compounds of the present invention are useful in the treatment of a number of human and veterinary pathogens, including aerobic as well as anaerobic Gram-positive and Gram-negative organisms.

### BACKGROUND TO THE INVENTION

Antibiotic resistance is a serious concern globally as it would result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention though being primarily effective against Gram-positive pathogens are also effective against certain Gram-negative pathogens.

Gram-positive pathogens, for example Staphylococci, Enterococci, Streptococci and Mycobacteria, are particularly important because of the development of resistant strains which are both difficult to treat and difficult to eradicate from the hospital environment once established. Examples of such strains are methicillin resistant staphylococcus (MRSA), methicillin resistant coagulase negative staphylococci (MRCNS), penicillin resistant Streptococcus pneumoniae and multiply resistant Enterococcus faecium and so on.

Antibacterial agents containing an oxazolidinone ring have been described in J. Med. Chem. 1992, 35, 2569-78 (Gregory W. A. et. al) and J. Med. Chem. 1992, 35, 1156-65 (Chung-Ho Park et. al). Also, U.S. Pat. Nos. 4,705,799 and 5,523,403 and EP0316594 disclose substituted phenyl-2-oxazolidinones. U.S. Pat. Nos. 4,948,801, 5,254,577 & 5,130,316 discloses arylbenzene oxazolidinyl compounds including substituted or unsubstituted phenyl and pyridyl groups. Heteroaryl-oxazolidinones having one to three atoms selected from the group consisting of oxygen, sulfur, nitrogen and oxygen are described in EP 0697412, 0694544, 0694543 & 0693491. Further, oxazolidinone derivatives useful as antibacterial agents are described in WO0218354, WO0218353, WO 0215980, WO 0220515, WO 0206278, WO 0181350, WO 0032599, WO 9807708, WO 9730981, WO 9721708, WO 9710235, WO 9709328, WO 9719089, WO 9710223, WO 9615130, WO 9613502, WO 9514684, WO 9507271, WO 9413649, WO9323384, WO 9309103, WO 9002744, U.S. Pat. No. 5,700,799, U.S. Pat. No. 4,801,600, U.S. Pat. No. 4,921,869, EP 0353781, EP 0316594, EP312000 etc.

Due to increase in antibiotic resistance there is a continuous need to develop more effective medicines suitable against such pathogenic organisms.

### SUMMARY OF THE INVENTION

The present invention describes a group of novel compounds useful as antibacterial agents. The novel compounds are defined by the general formula (I) below:

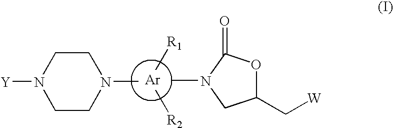

The compounds of the present invention are useful in the treatment of the human or animal body, as preventives and therapeutics for infectious diseases. The compounds of this invention have excellent antimicrobial action against various human and veterinary pathogens including but not limited to multiply-resistant staphylococci and streptococci, as well as anaerobic organisms including those of the bacteroides and clostridia species, and acid-fast Mycobacterium tuberculosis and Mycobacterium avium with better efficacy, potency and minimum toxic effects.

### OBJECTS OF THE INVENTION

The main objective of the present invention thus is to provide novel compounds of general formula (I), their analogs, their derivatives, their stereoisomers, their tautomeric forms, novel intermediates involved in their synthesis, their pharmaceutically acceptable salts, their pharmaceutically acceptable solvates and pharmaceutical compositions containing them or their mixtures suitable in the treatment of infectious diseases.

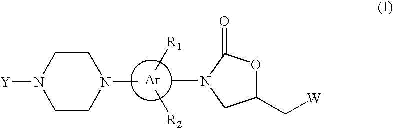

Another objective of the present invention is to provide a process for the preparation of novel compounds of general formula (I), their analogs, their derivatives, their stereoisomers, their polymorphs, their tautomeric forms, novel intermediates involved in their synthesis pharmaceutically acceptable salts, pharmaceutically acceptable solvates and pharmaceutical compositions containing them.

Yet another objective of the present invention is to provide pharmaceutical compositions containing compounds of general formula (I), their analogs, their derivatives, their stereoisomers, their polymorphs, their tautomeric forms, their pharmaceutically acceptable salts, solvates and their mixtures having pharmaceutically acceptable carriers, solvents, diluents, excipients and other media normally employed in their manufacture.

Still another objective of the present invention is to provide a method of treatment of antibiotic resistant pathogens, by administering a therapeutically effective amount of the compound of formula (I) or their pharmaceutically acceptable compositions to the mammals.

### DETAILED DESCRIPTION OF THE DESCRIPTION

The novel compounds of the present invention are defined by the general formula (I) below: 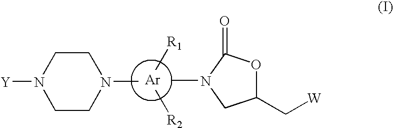
Where Ar represents an optionally substituted phenyl ring, five or six membered hetero aromatic ring which may be substituted or unsubstituted; R₁ & R₂ may be same or different and represent hydrogen, halogen, substituted or unsubstituted groups selected from alkyl, aralkyl, alkoxy, thio, amino, aminoalkyl, nitro, cyano, formyl, thioalkoxy, cycloalkyl, haloalkyl, haloalkoxy, groups;
Y represents the groups G₁, G₂ or G₃: 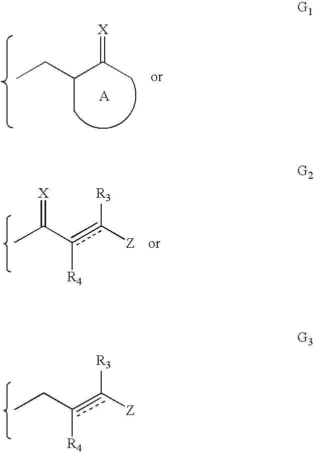
where R₃ & R₄ may be same or different and represent H, C₁-C₆ substituted or unsubstituted linear or branched alkyl group, halogen, hydroxy, cyano, haloalkyl, haloalkoxy, perhaloalkoxy, thio, substituted or unsubstituted groups selected from cycloalkyl, (C₁-C₁₂)alkoxy, cyclo(C₃-C₇)alkoxy, aryl, aryloxy, aralkyl, ar(C₁-C₁₂)alkoxy, acyl, acyloxy, carboxylic acid and its derivatives such as esters and amides, hydroxyalkyl, aminoalkyl, mono-substituted or di-substituted aminoalkyl, alkoxyalkyl, aryloxyalkyl, aralkoxyalkyl, (C₁-C₁₂)alkylthio, thio(C₁-C₁₂)alkyl & arylthio; X represents O, S or NR⁵ where R⁵ represents H or (un)substituted alkyl or aryl groups; A represents a (un)substituted, saturated or unsaturated or partially saturated single or fused ring moiety, optionally containing one or more heteroatoms selected from N, S, O; Z represents H, C₁-C₆ substituted or unsubstituted alkyl group, cyano, haloalkyl, haloalkoxy, perhaloalkoxy, substituted or unsubstituted groups selected from cycloalkyl, bicycloalkyl, (C₁-C₁₂)alkoxy, cyclo(C₃-C₇)alkoxy, aryl, aryloxy, aralkyl, ar(C₁-C₁₂)alkoxy, heterocyclyl, heteroaryl, heterocyclyl(C₁-C₂)alkyl, heteroar(C₁-C₁₂)alkyl, heteroaryloxy, heteroar(C₁-C₁₂)alkoxy, heterocycloxy, heterocyclylalkyloxy, acyl, acyloxy, acylamino, carboxylic acid and its derivatives such as esters and amides, hydroxyalkyl, aminoalkyl, mono-substituted or di-substituted aminoalkyl, alkoxyalkyl, aryloxyalkyl, aralkoxyalkyl, (C₁-C₁₂)alkylthio, thio(C₁-C₁₂)alkyl, arylthio, SOR₆ and SO₂R₆, where R₆ represents amino, optionally substituted groups selected from alkyl, aryl, heteroaryl, heterocyclyl groups; the dotted line ‘------’ represents either a bond or a no bond.

W represents OH, N₃, NH₂, NCS, OSO₂CH₃ or a moiety of general formula 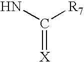
Wherein R₇ may be H, substituted or unsubstituted groups selected from amino, alkylamino, dialkylamino, aralkylamino, C₁-C₆alkoxy, C₁-C₁₂alkyl, aralkyl, C₃-C₁₂cycloalkyl, C₁-C₆thioalkyl, C₁-C₆haloalkyl, thioalkoxy, and X is selected from O, S, —NR₅ where R₅ represents H, or substituted or unsubstituted alkyl group or aryl groups.

Suitable rings representing A may be selected from but are not limited to 5-6 membered ring systems which may be single or fused and examples of ring moieties in G₁ may be cyclohexanone, cyclopentanone, α-tetralone, indanone, 6-methoxy-α-tetralone, 5-methoxy tetralone, indole, 5-methoxy indanone, dihydrobenzothiophenone and the like.

Suitable substituents on groups A & Z may be selected from cyano, nitro, halo, perhaloalkyl, carboxyl, hydrazino, azido, formyl, amino, thio, hydroxy, sulfonyl, or substituted or unsubstituted groups selected from alkyl which may be linear or branched; cycloalkyl, alkenyl, cycloalkenyl, alkynyl, hydrazinoalkyl, alkylhydrazido, hydroxylamino, acyl, acyloxy, acylamino, carboxyalkyl, haloalkyl, aminoalkyl, haloalkoxy, hydroxyalkyl, alkoxyalkyl, thioalkyl, alkylthio, alkylsulfinyl, alkylsulfonyl, alkylaminoalkyl, arylamino, alkylamino, aralkylamino, aralkoxy, haloaralkyl, aralkenyl, aryl, aralkyl, aryloxy, alkoxy, alkylcarbonyl, alkoxycarbonyl, aryloxycarbonyl, aralkoxycarbonyl, alkylcarbonylalkyl, alkoxycarbonylalkyl, 1-alkoxycarbonyloxy-alkyl, 1-cycloalkyloxycarbonyloxy-alkyl, carboxamidoalkyl, cyanoamidino, cyanoalkyl, aminocarbonylalkyl, N-aminocarbonylalkyl, N-arylaminocarbonyl, N-alkyl-N-arylaminocarbonyl, carboxyalkylaminocarboxy, N-alkylamino, N,N-dialkylamino, N-arylamino, N-aralkylamino, N-alkyl-N-aralkylamino, N-alkyl-N-arylamino, N-alkylaminoalkyl, N,N-dialkylaminoalkyl, N-arylaminoalkyl, N-aralkylaminoalkyl, N-alkyl-N-aralkylaminoalkyl, N-aralkyl-N-alkylaminoalkyl, N-alkyl-N-arylaminoalkyl, N,N-dialkylaminocarbonyl, N-alkyl-N-arylaminocarbonyl, N-alkyl-N-hydroxyaminocarbonyl, N-alkyl-N-hydroxyaminocarbonylalkyl, alkoxyalkyl, aryloxyalkyl, aralkoxyalkyl, arylthio, aralkylthio, alkoxycarbonyl, aminocarbonyl, alkoxycarbonylamino, cycloalkyl, bicycloalkyl, cycloalkoxy, bicycloalkenyl, heterocyclyl, heterocyclylalkyl, heteroaryl, heteroaralkyl, heteroaryloxy, heteroaralkoxy, heterocyclylalkyloxy, heterocycloalkoxycarbonyl, heteroaryloxycarbonyl, heteroaralkoxycarbonyl, RSO₂NH— and RSO₂O— groups wherein R represents alkyl, aryl, heteroaryl, aralkyl, heteroaralkyl, heterocyclyl, heterocyclylalkyl groups.

The term “alkyl” used herein, either alone or in combination with other radicals, denotes a linear or branched radical containing one to twelve carbons, such as methyl, ethyl, n-propyl, iso-propyl, n-butyl, sec-butyl, tert-butyl, amyl, t-amyl, n-pentyl, n-hexyl, iso-hexyl, heptyl, octyl and the like.

The term “alkenyl” used herein, either alone or in combination with other radicals, denotes a linear or branched radical containing one- to twelve carbons; such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl and the like. The term “alkenyl” includes dienes and trienes of straight and branched chains.

The term “alkynyl” used herein, either alone or in combination with other radicals, denotes a linear or branched radical containing one to twelve carbons, such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 3-hexynyl, 4-hexynyl, 5-hexynyl, and the like. The term “alkynyl” includes di- and tri-ynes.

The term “cyclo(C₃-C₇)alkyl” used herein, either alone or in combination with other radicals, denotes a radical containing three to seven carbons, such as cyclopropyl, cyclobutyl, cyclopentyl, cyclohexyl, cycloheptyl and the like.

The term “cyclo(C₃-C₇)alkenyl” used herein, either alone or in combination with other radicals, denotes a radical containing three to seven carbons, such as cyclopropenyl, 1-cyclobutenyl, 2-cylobutenyl, 1-cyclopentenyl, 2-cyclopentenyl, 3-cyclopentenyl, 1-cyclohexenyl, 2-cyclohexenyl, 3-cyclohexenyl, 1-cycloheptenyl, cycloheptadienyl, cycloheptatrienyl, and the like.

The term “alkoxy” used herein, either alone or in combination with other radicals, denotes a radical alkyl, as defined above, attached directly to an oxygen atom, such as methoxy, ethoxy, n-propoxy, iso-propoxy, n-butoxy, t-butoxy, iso-butoxy, pentyloxy, hexyloxy, and the like.

The term “alkenoxy” used herein, either alone or in combination with other radicals, denotes an alkenyl radical, as defined above, attached to an oxygen atom; such as vinyloxy, allyloxy, butenoxy, pentenoxy, hexenoxy, and the like.

The term “cyclo(C₃-C₇)alkoxy” used herein, either alone or in combination with other radicals, denotes a radical containing three to seven carbon atoms, such as cyclopropyloxy, cyclobutyloxy, cyclopentyloxy, cyclohexyloxy, cycloheptyloxy and the like.

The term “halo” or “halogen” used herein, either alone or in combination with other radicals, such as “haloalkyl”, “perhaloalkyl” etc refers to a fluoro, chloro, bromo or iodo group. The term “haloalkyl” denotes a radical alkyl, as defined above, substituted with one or more halogens; such as perhaloalkyl, more preferably, perfluoro(C₁-C₆)alkyl such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy, chloromethoxy, fluoroethoxy chloroethoxy groups, and the like. The term “perhaloalkoxy” denotes a perhaloalkyl radical, as defined above, directly attached to an oxygen atom, trifluoromethoxy, trifluoroethoxy, and the like.

The term “aryl” or “aromatic” used herein, either alone or in combination with other radicals, denotes an aromatic system containing one, two or three rings wherein such rings may be attached together in a pendant manner or may be fused, such as phenyl, naphthyl, tetrahydronaphthyl, indane, biphenyl, and the like. The term “aralkyl” denotes an alkyl group, as defined above, attached to an aryl, such as benzyl, phenethyl, naphthylmethyl, and the like. The term “aryloxy” denotes an aryl radical, as defined above, attached to an alkoxy group, such as phenoxy, naphthyloxy and the like, which may be substituted. The term “aralkoxy” denotes an arylalkyl moiety, as defined above, such as benzyloxy, phenethyloxy, naphthylmethyloxy, phenylpropyloxy, and the like, which may be substituted.

The term “heterocyclyl” or “heterocyclic” used herein, either alone or in combination with other radicals, denotes saturated, partially saturated and unsaturated ring-shaped radicals, the heteroatoms selected from nitrogen, sulfur and oxygen. Examples of saturated heterocyclic radicals include aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, and the like; examples of partially saturated heterocyclic radicals include dihydrothiophene, dihydropyran, dihydrofuran, dihydrothiazole, and the like.

The term “heteroaryl” or “heteroaromatic” used herein, either alone or in combination with other radicals, denotes unsaturated 5 to 6 membered heterocyclic radicals containing one or more hetero atoms selected from O, N or S, attached to an aryl group, such as pyridyl, thienyl, furyl, pyrrolyl, oxazolyl, thiazolyl, imidazolyl, isoxazolyl, oxadiazolyl, tetrazolyl, benzopyranyl, benzofuranyl, benzothienyl, indolinyl, indolyl, quinolinyl, pyrimidinyl, pyrazolyl, quinazolinyl, pyrimidonyl, benzoxazinyl, benzoxazinonyl, benzothiazinyl, benzothiazinonyl, benzoxazolyl, benzothizaolyl, benzimidazolyl, and the like.

The term “heterocyclyl(C₁-C₁₂)alkyl” used herein, either alone or in combination with other radicals, represents a heterocyclyl group, as defined above, substituted with an alkyl group of one to twelve carbons, such as pyrrolidinealkyl, piperidinealkyl, morpholinealkyl thiomorpholinealkyl, oxazolinealkyl, and the like, which may be substituted. The term “heteroaralkyl” used herein, either alone or in combination with other radicals, denotes a heteroaryl group, as defined above, attached to a straight or branched saturated carbon chain containing 1 to 6 cartons, such as (2-furyl)methyl, (3-furyl)methyl, (2-thienyl)methyl, (3-thienyl)methyl, (2-pyridyl)methyl, 1-methyl-1-(2-pyrimidyl)ethyl and the like. The terms “heteroaryloxy”, “heteroaralkoxy”, “heterocycloxy”, “heterocylylalkoxy” denotes heteroaryl, heteroarylalkyl, heterocyclyl, heterocylylalkyl groups respectively, as defined above, attached to an oxygen atom.

The term “acyl” used herein, either alone or in combination with other radicals, denotes a radical containing one to eight carbons such as formyl, acetyl, propanoyl, butanoyl, iso-butanoyl, pentanoyl, hexanoyl, heptanoyl, benzoyl and the like, which may be substituted.

The term “acyloxy” used herein, either alone or in combination with other radicals, denotes a radical acyl, as defined above, directly attached to an oxygen atom, such as acetyloxy, propionyloxy, butanoyloxy, iso-butanoyloxy, benzoyloxy and the like.

The term “acylamino” used herein, either alone or in combination with other radicals, denotes an acyl group as defined earlier, may be CH₃CONH, C₂H₅CONH, C₃H₇CONH, C₄H₉CONH, C₆H₅CONH and the like, which may be substituted.

The term “mono-substituted amino” used herein, either alone or in combination with other radicals, denotes an amino group, substituted with one group selected from (C₁-C₆)alkyl, substituted alkyl, aryl, substituted aryl or arylalkyl groups. Examples of monoalkylamino group include methylamine, ethylamine, n-propylamine, n-butylamine, n-pentylamine and the like.

The term “disubstituted amino” used herein, either alone or in combination with other radicals, denotes an amino group, substituted with two radicals that may be same or different selected from (C₁-C₆)alkyl, substituted alkyl, aryl, substituted aryl, or arylalkyl groups, such as dimethylamino, methylethylamino, diethylamino, phenylmethyl amino and the like.

The term “arylamino” used herein, either alone or in combination with other radicals, denotes an aryl group, as defined above, linked through amino having a free valence bond from the nitrogen atom, such as phenylamino, naphthylamino, N-methyl anilino and the like.

The term “aralkylamino” used herein, either alone or in combination with other radicals, denotes an arylalkyl group as defined above linked through amino having a free valence bond from the nitrogen atom e.g. benzylamino, phenethylamino, 3-phenylpropylamino, 1-napthylmethylamino, 2-(1-napthyl)ethylamino and the like.

The term “oxo” or “carbonyl” used herein, either alone (—C═O—) or in combination with other radicals, such as “alkylcarbonyl”, denotes a carbonyl radical (—C═O—) substituted with an alkyl radical such as acyl or alkanoyl, as described above.

The term “carboxylic acid” used herein, alone or in combination with other radicals, denotes a —COOH group, and includes derivatives of carboxylic acid such as esters and amides. The term “ester” used herein, alone or in combination with other radicals, denotes —COO— group, and includes carboxylic acid derivatives, where the ester moieties are alkoxycarbonyl, such as methoxycarbonyl, ethoxycarbonyl, and the like, which may be substituted; aryloxycarbonyl group such as phenoxycarbonyl, napthyloxycarbonyl, and the like, which may be substituted; aralkoxycarbonyl group such as benzyloxycarbonyl, phenethyloxycarbonyl, napthylmethoxycarbonyl, and the like, which may be substituted; heteroaryloxycarbonyl, heteroaralkoxycarbonyl, wherein the heteroaryl group, is as defined above, which may be substituted; heterocyclyloxycarbonyl, where the heterocyclic group, as defined earlier, which may be substituted.

The term “amide” used herein, alone or in combination with other radicals, represents an aminocarbonyl radical (H₂N—C═O—), wherein the amino group is mono- or di-substituted or unsubstituted, such as methylamide, dimethylamide, ethylamide, diethylamide, and the like. The term “aminocarbonyl” used herein, either alone or in combination with other radicals, with other terms such as “aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, substituted or unsubstituted. The terms “N-alkylaminocarbonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote amiocarbonyl radicals substituted, respectively, with one aryl radical, or one alkyl, and one aryl radical. The term “aminocarbonylalkyl” includes alkyl radicals substituted with aminocarbonyl radicals.

The term “hydroxyalkyl” used herein, either alone or in combination with other radicals, denotes an alkyl group, as defined above, substituted with one or more hydroxy radicals, such as hydroxymethyl, hydroxyethyl, hydroxypropyl, hydroxybutyl, hydroxypentyl, hydroxyhexyl and the like.

The term “aminoalkyl” used herein, alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino.

The term “alkoxyalkyl” used herein, alone or in combination with other radicals, denotes an alkoxy group, as defined above, attached to an alkyl group, such as methoxymethyl, ethoxymethyl, methoxyethyl, ethoxyethyl and the like. The term “aryloxyalkyl” used herein, alone or in combination with other radicals, includes phenoxymethyl, napthyloxymethyl, and the like. The term “aralkoxyalkyl” used herein, alone or in combination with other radicals, includes C₆H₅CH₂OCH₂, C₆H₅CH₂OCH₂CH₂, and the like.

The term “(C₁-C₁₂)alkylthio” used herein, either alone or in combination with other radicals, denotes a straight or branched or cyclic monovalent substituent comprising an alkyl group of one to twelve carbon atoms, as defined above, linked through a divalent sulfur atom having a free valence bond from the sulfur atom, such as methylthio, ethylthio, propylthio, butylthio, pentylthio and the like. Examples of cyclic alkylthio are cyclopropylthio, cyclobutylthio, cyclopentylthio, cyclohexylthio and the like, which may be substituted.

The term “thio(C₁-C₁₂)alkyl” used herein, either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be substituted.

The term “arylthio” used herein, either alone or in combination with other radicals, denotes an aryl group, as defined above, linked through a divalent sulfur atom, having a free valence bond from the sulfur atom such as phenylthio, napthylthio and the like.

The term “(C₁-C₁₂)alkoxycarbonylamino” used herein, alone or in combination with other radicals, denotes an alkoxycarbonyl group, as defined above, attached to an amino group, such as methoxycarbonylamino, ethoxycarbonylamino, and the like. The term “aryloxycarbonylamino” used herein, alone or in combination with other radicals, denotes an aryloxycarbonyl group, as defined above, attached to the an amino group, such as C₆H₅OCONH, C₆H₅OCONCH₃, C₆H₅OCONC₂H₅, C₆H₄(CH₃O)CONH, C₆H₄(OCH₃)OCONH, and the like. The term “aralkoxycarbonylamino” used herein, alone or in combination with other radicals, denotes an aralkoxycarbonyl group, as defined above, attached to an amino group C₆H₅CH₂OCONH, C₆H₅CH₂CH₂CH₂OCONH, C₆H₅CH₂OCONHCH₃, C₆H₅CH₂OCONC₂H₅, C₆H₄(CH₃)CH₂OCONH, C₆H₄(OCH₃)CH₂OCONH, and the like.

The term “aminocarbonylamino”, “alkylaminocarbonylamino”, “dialkylaminocarbonylamino” used herein, alone or in combination with other radicals, denotes a carbonylamino (—CONH₂) group, attached to amino(NH₂), alkylamino group or dialkylamino group respectively, where alkyl group is as defined above.

The term “hydrazino” used herein, either alone or in combination with other radicals, denotes —NHNH—, suitably substituted with other radicals, such as alkyl hydrazino, where an alkyl group, as defined above is attached to a hydrazino group.

The term “alkoxyamino” used herein, alone or in combination with other radicals, denotes an alkoxy group, as defined above, attached to an amino group. The term “hydroxyamino” used herein, alone or in combination with other radicals, denotes —NHOH moiety, and may be substituted.

The term “sulfenyl” or “sulfenyl and its derivatives” used herein, alone or in combination with other radicals, denotes a bivalent group, —SO— or RSO, where R is substituted or unsubstituted alkyl, aryl, heteroaryl, heterocyclyl, and the like.

The term “sulfonyl” or “sulfones and its derivatives” used herein, either alone or in combination with other radicals, with other terms such as alkylsulfonyl, denotes divalent radical —SO₂—, or RSO₂—; where R is substituted or unsubstituted groups selected from alkyl, aryl, heteroaryl, heterocyclyl, and the like. “Alkylsulfonyl” denotes alkyl radicals, as defined above, attached to a sulfonyl radical, such as methylsulfonyl, ethylsulfonyl, propylsulfonyl and the like. The term “arylsulfonyl” used herein, either alone or in combination with other radicals, denotes aryl radicals, as defined above, attached to a sulfonyl radical, such as phenylsulfonyl and the like.

Suitable groups and substituents on the groups may be selected from those described anywhere in the specification.

Particularly useful compounds of the present invention are:
(S)-N-[3-(3-Fluoro-4-{4-[3-(4-hydroxyphenyl)-acryloyl]-piperazin-1-yl]-phenyl)-2-oxo-oxazolidin-5-yl methyl]acetamide;
(S)-N-[3-(3-Fluoro-4-{4-[3-(4-hydroxyphenyl)-acryloyl]-piperazin-1-yl]-phenyl)-2-oxo-oxazolidin-5-yl methyl]thioacetamide;
(S)-N-[3-(3-Fluoro-4-{4-[3-(4-hydroxyphenyl)-acryloyl]-piperazin-1-yl]-phenyl)-2-oxo-oxazolidin-5-yl methyl thiourea;
(S)-N-[3-(3-Fluoro-4-{4-[3-(3-hydroxyphenyl)-acryloyl]-piperazin-1-yl]-phenyl)-2-oxo-oxazolidin-5-yl methyl]acetamide;
(S)-N-[3-(3-Fluoro-4-{4-[3-(3-hydroxyphenyl)-acryloyl]-piperazin-1-yl]-phenyl)-2-oxo-oxazolidin-5-yl methyl]thioacetamide;
(S)-N-[3-{4-(4-(3-Benzo[1,3]-dioxol-5-yl-acryloyl)-piperazin-1-yl}-3-fluorophenyl]-2-oxo-oxazolidin-5-yl methyl]acetamide;
(S)-N-[3-{4-(4-(3-Benzo[1,3]-dioxol-5-yl-acryloyl)-piperazin-1-yl}-3-fluorophenyl]-2-oxo-oxazolidin-5-yl methyl]thioacetamide;
(S)-N-[3-{4-(4-(3-Benzo[1,3]-dioxol-5-yl-acryloyl)-piperazin-1-yl}-3-fluorophenyl]-2-oxo-oxazolidin-5-yl methyl thiourea;
(S)-N-[3-(3-Fluoro-4-{4-[3-(thiophen-3-yl)-acryloyl]-piperazinyl]-phenyl)-2-oxo-oxazolidin-5-yl methyl]acetamide;
(S)-N-[3-(3-Fluoro-4-{4-[3-(thiophen-2-yl)-acryloyl]-piperazinyl]-phenyl)-2-oxo-oxazolidin-5-yl methyl]acetamide;
(S)-N-[3-(3-Fluoro-4-{4-[3-(thiophen-2-yl)-acryloyl]-piperazinyl]-phenyl)-2-oxo-oxazolidin-5-yl methyl]thioacetamide;
(S)-N-[3-(3-Fluoro-4-{4-[3-(thiophen-2-yl)-acryloyl]-piperazinyl]-phenyl)-2-oxo-oxazolidin-5-yl methyl]thiourea,
(S)-N-[3-(3-Fluoro-4-{4-[3-(thiophen-2-yl)-acryloyl]-piperazinyl]-phenyl)-2-oxo-oxazolidin-5-yl methyl]thiocarbamate;
(S)-N-[3-(3-Fluoro-4-{4-[3-(1H-indol-3-yl)-acryloyl]-piperazin-1-yl]-phenyl)-2-oxo-oxazolidin-5-yl methyl]acetamide;
(S)-N-[3-(3-Fluoro-4-{4-{3-(1H-indol-3-yl)-acryloyl]-piperazin-1-yl]-phenyl)-2-oxo-oxazolidin-5-yl methyl]thioacetamide;
(S)-N-[3-(3-Fluoro-4-{4-[3-(1H-indol-3-yl)-acryloyl]-piperazin-1-yl]-phenyl)-2-oxo-oxazolidin-5-yl methyl]thiourea;
(S)-N-[3-(3-Fluoro-4-{4-[3-(furan-2-yl)-acryloyl]-piperazin-1-yl]-phenyl)-2-oxo-oxazolidin-5-yl methyl]acetamide;
(S)-N-[3-(3-Fluoro-4-{4-[3-(furan-2-yl)-acryloyl]-piperazin-1-yl]-phenyl)-2-oxo-oxazolidin-5-yl methyl]thioacetamide;
(S)-N-[3-(3-Fluoro-4-{4-[3-(furan-2-yl)-acryloyl]-piperazin-1-yl]-phenyl)-2-oxo-oxazolidin-5-yl methyl]thiourea;
(S)-N-[3-(3-Fluoro-4-{4-[3-(pyridin-3-yl)-acryloyl]-piperazin-1-yl]-phenyl)-2-oxo-oxazolidin-5-yl methyl]acetamide;
(S)-N-[3-(3-Fluoro-4-{4-[3-(pyridin-3-yl)-acryloyl]-piperazin-1-yl]-phenyl)-2-oxo-oxazolidin-5-yl methyl]thioacetamide;
(S)-N-[3-(3-Fluoro-4-{4-[3-(pyridin-4-yl)-acryloyl]-piperazin-1-yl]-phenyl)-2-oxo-oxazolidin-5-yl methyl]acetamide;
(S)-N-[3-(3-Fluoro-4-{4-[3-(pyridin-4-yl)-acryloyl]-piperazin-1-yl]-phenyl)-2-oxo-oxazolidin-5-yl methyl]thioacetamide;
(S)-N-[3-(3-Fluoro-4-{4-[3-(pyridin-4-yl)-acryloyl]-piperazin-1-yl]-phenyl)-2-oxo-oxazolidin-5-yl methyl]thiourea;
(S)-N-[3-(3-Fluoro-4-{4-[3-phenyl-propanoyl]-piperazin-1-yl]-phenyl)-2-oxo-oxazolidin-5-yl methyl]acetamide;
(S)-N-[3-(3-Fluoro-4-{4-[3-phenyl-propanoyl]-piperazin-1-yl]-phenyl)-2-oxo-oxazolidin-5-yl methyl]thioacetamide;
(S)-N-[3-(3-Fluoro-4-{4-[3-(4-fluorophenyl)-acryloyl]-piperazin-1-yl]-phenyl)-2-oxo-oxazolidin-5-yl methyl]acetamide;
(S)-N-[3-(3-Fluoro-4-{4-[3-(4-fluorophenyl)-acryloyl]-piperazin-1-yl]-phenyl)-2-oxo-oxazolidin-5-yl methyl]thioacetamide;
(S)-N-[3-(3-Fluoro-4-{4-[3-(4-fluorophenyl)-acryloyl]-piperazin-1-yl]-phenyl)-2-oxo-oxazolidin-5-yl methyl]thiourea;
(S)-N-[3-(3-Fluoro-4-{4-[3-(4-fluorophenyl)-acryloyl]-piperazin-1-yl]-phenyl)-2-oxo-oxazolidin-5-yl methyl]thiocarbamate;
(S)-N-[3-(3-Fluoro-4-{4-[3-phenyl acryloyl]-piperazin-1-yl]-phenyl)-2-oxo-oxazolidin-5-yl methyl]acetamide;
(S)-N-[3-(3-Fluoro-4-{4-[3-phenyl acryloyl]-piperazin-1-yl]-phenyl)-2-oxo-oxazolidin-5-yl methyl]thioacetamide;
(S)-N-[3-(3-Fluoro-4-{4-[3-phenyl acryloyl]-piperazin-1-yl]-phenyl)-2-oxo-oxazolidin-5-yl methyl]thiourea;
(S)-N-[3-(3-Fluoro-4-{4-[3-(4-methoxyphenyl)acryloyl)-piperazin-1-yl]-phenyl)-2-oxo-oxazolidin-5-yl methyl]acetamide;
(S)-N-[3-(3-Fluoro-4-{4-[3-(4-methoxyphenyl)acryloyl)-piperazin-1-yl]-phenyl)-2-oxo-oxazolidin-5-yl methyl]thioacetamide;
(S)-N-[3-(3-Fluoro-4-{4-[3-(4-methoxyphenyl)acryloyl)-piperazin-1-yl]-phenyl)-2-oxo-oxazolidin-5-yl methyl]thiourea;
(S)-N-[3-(3-Fluoro-4-{4-[3-(4-acetoxyphenyl)acryloyl)-piperazin-1-yl]-phenyl)-2-oxo-oxazolidin-5-yl methyl]acetamide;
(S)-N-[3-(3-Fluoro-4-{4-[3-(4-acetoxyphenyl)acryloyl)-piperazin-1-yl]-phenyl)-2-oxo-oxazolidin-5-yl methyl]thioacetamide;
(S)-N-[3-(3-Fluoro-4-{4-[3-(4-acetoxyphenyl)acryloyl)-piperazin-1-yl]-phenyl)-2-oxo-oxazolidin-5-yl methyl]thiourea;
(S)-N-[3-(3-Fluoro-4-{4-[3-furan-3-yl-acryloyl)-piperazin-1-yl]-phenyl)-2-oxo-oxazolidin-5-yl methyl]acetamide:
(S)-N-[3-(3-Fluoro-4-{4-[3-(3,4-difluorophenyl)-acryloyl)-piperazin-1-yl]-phenyl)-2-oxo-oxazolidin-5-yl methyl]acetamide;
(S)-N-[3-(3-Fluoro-4-{4-[3-(3,4-difluorophenyl)-acryloyl)-piperazin-1-yl]-phenyl)-2-oxo-oxazolidin-5-yl methyl]thioacetamide;
(S)-N-[3-(3-Fluoro-4-{4-[3-(3,4-difluorophenyl)-acryloyl)-piperazin-1-yl]-phenyl)-2-oxo-oxazolidin-5-yl methyl]thioacetamide;
Methanesulfonic acid 4-[3-(4-{4-[5-(acetyl aminomethyl)-2-oxo-oxazolidin-3-yl]-2-fluorophenyl}piperazin-1-yl]-3-oxo-propenyl]-phenyl ester;
(S)-N-[3-(3-Fluoro-4-{4-[3-(4-methylsulfanyl-phenyl)acryloyl)-piperazin-1-yl]-phenyl)-2-oxo-oxazolidin-5-yl methyl]acetamide;
(S)-N-[3-(4-{4-[3-(3,4-dihydroxyphenyl)-acryloyl)-piperazin-1-yl]-3-fluorophenyl)-2-oxo-oxazolidin-5-yl methyl]acetamide;
(S)-N-[3-(4-{4-[3-biphenyl-4-yl-acryloyl)-piperazin-1-yl)]-3-fluorophenyl)-2-oxo-oxazolidin-5-yl methyl]acetamide
(S)-N-[3-(4-{4-but-2-enoyl-piperazin-1-yl]-3-fluorophenyl)-2-oxo-oxazolidin-5-yl methyl]acetamide;
(S)-N-[3-(4-{4-acryloyl-piperazin-1-yl]-3-fluorophenyl)-2-oxo-oxazolidin-5-yl methyl]acetamide;
(S)-N-[3-(3-Fluoro-4-{4-[2-methylacryloyl-piperazin-1-yl]-phenyl)-2-oxo-oxazolidin-5-yl methyl]acetamide;
(S)-N-[3-(-4-{4-[3-(4-benzyloxy-phenyl)-acryloyl)-piperazin-1-yl]-3-fluorophenyl)-2-oxo-oxazolidin-5-yl methyl]thiourea;
(S)-N-[3-(4-{4-[3-(4-nitrophenyl)-acryloyl)-piperazin-1-yl]-3-fluorophenyl)-2-oxo-oxazolidin-5-yl methyl]acetamide;
Carbonic acid-1-{4-[3-(4-{4-[5-(acetylamino-methyl)-2-oxo-oxazolidin-3-yl]-2-fluorophenyl}-piperazin-1-yl)-3-oxo-propenyl]-phenoxy}-ethyl ether cyclohexyl ester;
(S)-N-[3-(4-{4-[3-(4-aminophenyl)-acryloyl)-piperazin-1-yl]-3-fluorophenyl)-2-oxo-oxazolidin-5-yl methyl]acetamide;
(S)-N-[3-(4-{4-[3-(3,4-diacetoxy-phenyl)-acryloyl)-piperazin-1-yl]-3-fluorophenyl)-2-oxo-oxazolidin-5-yl methyl]acetamide;
(S)-N-[3-(4-{4-[3-benzo[1,3]-dioxol-5-yl acryloyl)-piperazin-1-yl]-3-fluorophenyl)-2-oxo-oxazolidin-5-yl methyl]thiocarbamate;
(S)-N-[3-(3-Fluoro-4-[4-(4-oxo-4-phenyl-but-2-enoyl)-piperazin-1-yl]-phenyl)-2-oxo-oxazolidin-5-yl methyl]acetamide;
(S)-N-[3-(3-Fluoro-4-[4-(4-(4-methoxyphenyl)-4-oxo-but-2-enoyl)-piperazin-1-yl]-phenyl)-2-oxo-oxazolidin-5-yl methyl]acetamide;
(S)-N-[3-(3-Fluoro-4-[4-(4-(4-methoxyphenyl)-4-oxo-but-2-enoyl)-piperazin-1-yl]-phenyl)-2-oxo-oxazolidin-5-yl methyl]thioacetamide;
(S)-N-[3-{4-[4-(4-(4-acetylaminophenyl)-4-oxo-but-2-enoyl)-piperazin-1-yl]-3-fluorophenyl)-2-oxo-oxazolidin-5-yl methyl]acetamide;
(S)-N-[3-(3-Fluoro-4-[4-(4-(4-acetylaminophenyl)acryloyl)-piperazin-1-yl]-3-fluorophenyl)-2-oxo-oxazolidin-5-yl methyl]acetamide;
(S)-N-[3-(3-Fluoro-4-[4-(3-cyclohexyl)-acryloyl-piperazin-1-yl]-3-fluorophenyl)-2-oxo-oxazolidin-5-yl methyl]acetamide;
Acetic acid-2-(4-{4-[5-(acetylaminomethyl)-2-oxo-oxazolidin-3-yl]-2-fluorophenyl][-piperazinyl-1-carbonyl-7-amino-3-oxo-5-thia-1-aza-bicyclo-[4.2.0]-oct-2-en-3-yl-methyl ester;
2,2-Dimethyl-propanoic acid-4-(3-(4-{4-[5-(acetylaminomethyl)-2-oxo-oxazolidin-3-yl]-2-fluorophenyl}piperazinyl-1-yl)-3-oxo-propenyl]-phenyl ester;
Carbonic acid-1-{4-[3-(4-{4-[5-(acetylaminomethyl)-2-oxo-oxazolidin-3-yl]-2-fluorophenyl][-piperizinyl-1-yl)-3-oxo-propenyl]-phenyl ester;
(S)-N-[3-(3-Fluoro-4-[4-(3-(5-nitrofuran-2-yl)-acryloyl-piperazin-1-yl]-3-phenyl)-2-oxo-oxazolidin-5-yl methyl]acetamide;
(S)-N-[3-(3-Fluoro-4-[4-(6-methoxy-1-oxo-1,2,3,4 tetrahydronaphthalen-2-yl methyl)-piperazin-1-yl]-phenyl)-2-oxo-oxazolidin-5-yl methyl]acetamide;
(S)-N-[3-(3-Fluoro-4-[4-(1-oxo-1,2,3,4 tetrahydronaphthalen-2-yl methyl)-piperazin-1-yl]-3-phenyl)-2-oxo-oxazolidin-5-yl methyl]acetamide;
(S)-N-[3-(3-Fluoro-4-[4-(5-methoxy-1-oxo-indan-2-yl-methyl)-piperazin-1-yl]-3-phenyl)-2-oxo-oxazolidin-5-yl methyl]acetamide;
(S)-N-[3-(3-Fluoro-4-[4-(2-oxo-cyclohexylmethyl)-piperazin-1-yl]-3-phenyl)-2-oxo-oxazolidin-5-yl methyl]acetamide;
(S)-N-[3-(3-Fluoro-4-[4-(6-methoxy-1-oxo-1,2,3,4 tetrahydronaphthalen-2-yl methyl)-piperazin-1-yl]-3-phenyl)-2-oxo-oxazolidin-5-yl methyl]thioacetamide;
(S)-N-[3-(3-Fluoro-4-[4-(5-methoxy-1-oxo-indan-2-yl-methyl)-piperazin-1-yl]-3-phenyl)-2-oxo-oxazolidin-5-yl methyl]thioacetamide;
(S)-N-[3-(3-Fluoro-4-[4-(1-hydroxyimino-6-methoxy-1,2,3,4 tetrahydronaphthalen-1-yl methyl)-piperazin-1-yl]-phenyl)-2-oxo-oxazolidin-5-yl methyl]acetamide;
(S)-N-[3-(3-Fluoro-4-[4-(4-methyl-1-oxo-1,2,3,4 tetrahydronaphthalen-2-yl methyl)-piperazin-1-yl]-phenyl)-2-oxo-oxazolidin-5-yl methyl]thioacetamide;
Trans-(S)-N-(3-{3-Fluoro-4-[4-(3-1H-pyrrol-2-yl-acryloyl)-piperazin-1-yl]-phenyl}-2-oxo-oxazolidin-5-yl-methyl)acetamide.
Cis-(S)-N-(3-{3-Fluoro-4-[4-(3-1H-pyrrol-2-yl-acryloyl)-piperazin-1-yl]-phenyl}-2-oxo-oxazolidin-5-yl-methyl)acetamide.
(S)-5-[3-(4-{4-[5-Acetylamino-methyl)-2-oxo-oxazolin-3-yl]-2-fluoro-phenyl}-piperazin-1-yl)-3-oxo-propenyl]-furan-2-carboxlic acid sodium salt
(S)-5-[3-(4-{4-[5-(Acetylamino-methyl)-2-oxo-oxazolin-3-yl]-2-fluoro-phenyl}-piperazin-1-yl)-3-oxo-propenyl]-furan-2-carboxlic acid.
(S)-N-[3-(3-Fluoro-4-{4-[3-(5-hydroxymethyl-furan-2-yl)-acryloyl]-piperazin-1-yl}-phenyl)-2-oxo-oxazolidin-5-yl methyl]acetamide.
(S)-N-[3-(3-Fluoro-4-{4-[3-(4-methanesulfonyl-phenyl)-acryloyl]-piperazin-1-yl}-phenyl)-2-oxo-oxazolidin-5-yl methyl]acetamide.
(S)-4-(4-{4-[5-(Aceylamino-methyl)-2-oxo-oxazolidin-3-yl]-2-fluoro-phenyl}-piperazin-1-yl)-4-oxo-but-2-enoic acid.
(S)-N-[3-(3-Fluoro-4-{4-[3-(5-formyl-furan-2-yl)-acryloyl]-piperazin-1-yl}-phenyl)-2-oxo-oxazolidin-5-yl methyl]acetamide.
(S)-Acetic acid-5-[3-(4-{4-[5-(Acetylamino-methyl)-2-oxo-oxazolin-3-yl]-2-fluoro-phenyl}-piperazin-1-yl)-3-oxo-propenyl]-furan-2-yl methyl ester.
(S)-4-(4-{4-[5-(Aceylamino-methyl)-2-oxo-oxazolidin-3-yl]-2-fluoro-phenyl}-piperazin-1-yl)-4-oxo-but-2-enoic acid sodium salt.
(S)-N-[3-(3-Fluoro-4-{4-[3-(5-methyl-furan-2-yl)-acryloyl]-piperazin-1-yl}-phenyl)-2-oxo-oxazolidin-5-yl methyl]acetamide.
(S)-N-[3-(3-Fluoro-4-{4-propynoyl-piperazin-1-yl]-phenyl)-2-oxo-oxazolidin-5-yl methyl]acetamide;
(S)-N-[3-(3-Fluoro-4-{4-(4-hydroxy-but-2-enoyl)-piperazin-1-yl]-phenyl)-2-oxo-oxazolidin-5-yl methyl]acetamide;
(S)-N-[3-(3-Fluoro-4-{4-(4-bromo-but-2-enoyl)-piperazin-1-yl]-phenyl)-2-oxo-oxazolidin-5-yl methyl]acetamide;
2-[4-(4-{5-(acetylamino-methyl)-2-oxo-oxazolidin-3-yl}-2-fluorophenyl)-piperazin-1-carbonyl]-3-phenyl-acrylic acid methyl ester;
2-[4-(4-{5-(acetylamino-methyl)-2-oxo-oxazolidin-3-yl}-2-fluorophenyl)-piperazin-1-carbonyl]-3-phenyl-acrylic acid;
2-[4-(4-{5-(acetylamino-methyl)-2-oxo-oxazolidin-3-yl}-9-fluorophenyl)-piperazin-1-carbonyl]-3-furane acrylic acid methyl ester;
2-[4-(4-{5-(acetylamino-methyl)-2-oxo-oxazolidin-3-yl}-2-fluorophenyl)-piperazin-1-carbonyl]-3-furane-acrylic acid;

The compounds of general formula (I) may be prepared by one or more routes or combinations of reactions given below and outlined in detail. The method comprises:

i) Route 1:

by reacting a compound of formula (1a) with a compound of formula (1b) 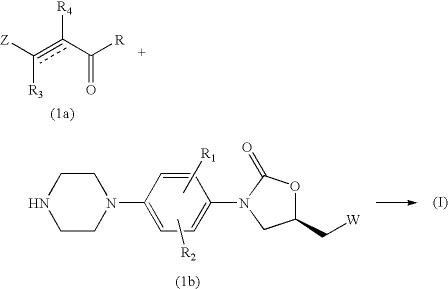
where R represents OH, halide or an acyloxy group, to yield compound of formula (I)
where Y represents G₂ and all symbols are as defined earlier.
ii) Route 2:
by reacting a compound of formula (1c) with a compound of formula (1b) 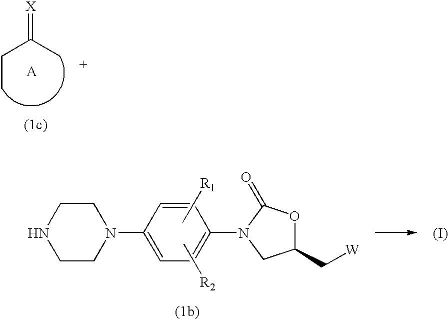
to yield compounds of formula (I); where Y represents G₁ and all symbols are as defined earlier.
iii) Route 3:

A process which comprises:
a) reaction of a compound of formula (1a) with a compound of formula (1d) to yield 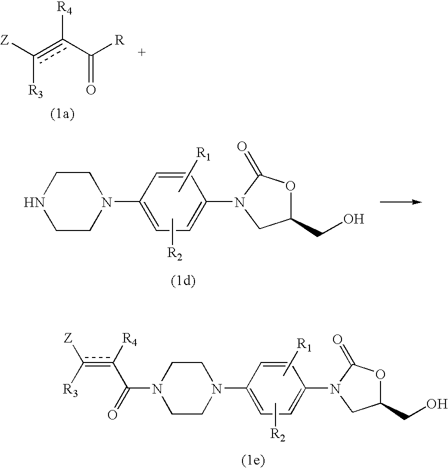
   (1e),
   where all symbols are as defined earlier;
b) Converting a compound of formula (1e) to (1f) where L represents a leaving group such as —OMs, —OTs, halides etc. and all other symbols are as defined earlier; 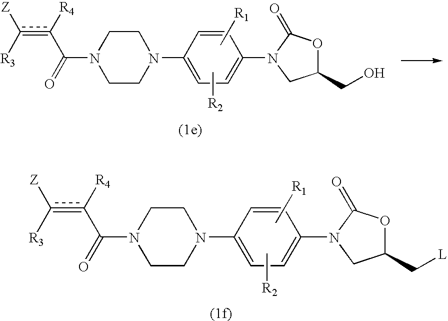
c) Converting a compound (1f) to (1g), where all symbols are as defined earlier; 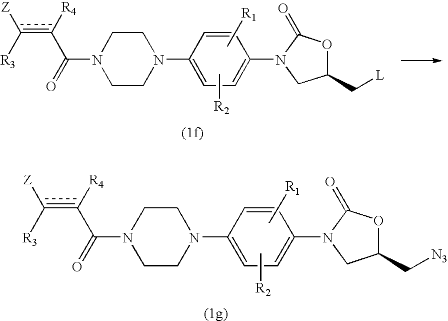
d) Converting a compound (1g) to (1h), where all symbols are as defined earlier; 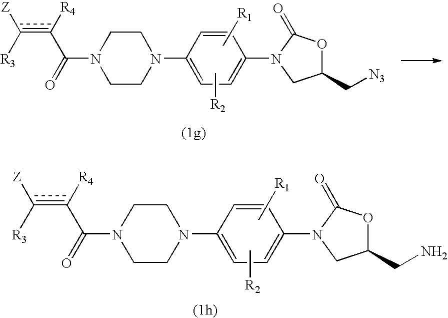
e) Converting a compound (1h) to (1i), where all symbols are as defined earlier; 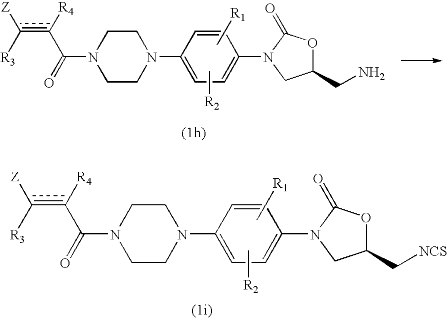
f) Converting a compound (1i) to (1j), where all symbols are as defined earlier;

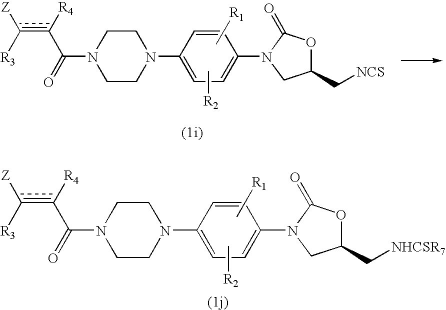

Alternatively,
g) Converting a compound (1i) to (1k) where all symbols are as defined earlier;

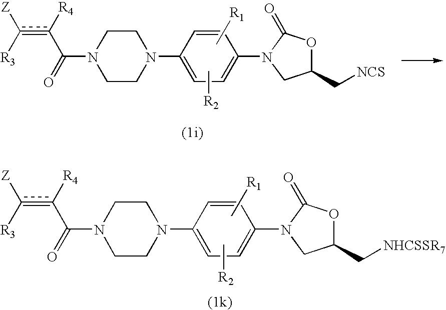

Alternatively
h) Converting a compound (1i) to (1l), where all symbols are as defined earlier;

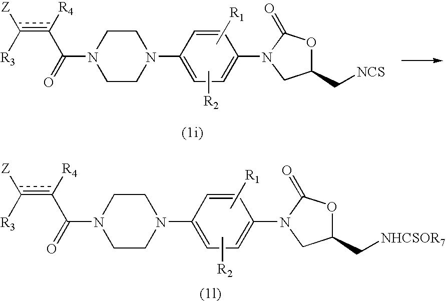

Compounds of formula (Ie), (Ig), (Ih), (Ii), (Ij), (Ik), (Il) represent compounds of formula (I), where all symbols are as defined earlier and W represents OH, N₃, NH₂, NCS, NHCSR₇, NHCSSR₇, NHCSOR₇ respectively and Y represents G₂ with X=O;

iv) Route 4:

Reacting a compound of formula (1m) with a compound of formula (1b) to give compound of formula (1n), where all symbols are as defined earlier; The compound (In) is a compound of formula (I), where Y represents G₃.

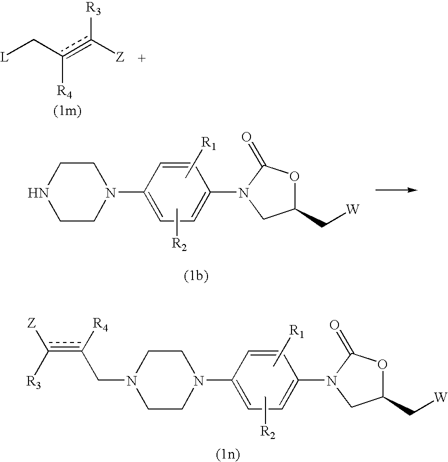

The reactions described in the processes outlined above may be performed by using the methods described herein:

Route 1:

Compounds of general formula I may be obtained from compound of general formula (Ia) by coupling with compound of general formula (Ib), employing different coupling agents depending upon the nature of (Ia) such as acid chlorides or mixed anhydrides corresponding to (Ia). Bases such as Na₂CO₃, K₂CO₃ and the like; organic bases like triethylamine, pyridine, diisopropylethylamine and the like; solvents such as acetone, THF may be used. Temperature in the range of −20° C. to reflux temperature of the solvent may be used. If (Ia) is an acid, suitable coupling agents like DCC, HOBT and the like may be used. Solvents such as dichloromethane, chloroform may be used. 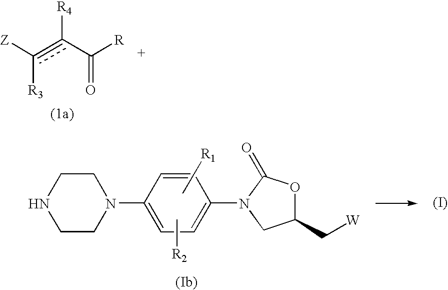
Route 2:

Compounds of general formula I may be obtained by reacting compounds of general formula (Ic) with compounds of general formula (Ib), in presence of formaldehyde or paraformaldehyde and HCl in methanol or ethereal HCl or 1,3 dioxalane and conc. HCl. Solvents such as THF, Diethyl ether may be used. Temperature in the range of 0° C. to reflux temperature of the solvent may be used. 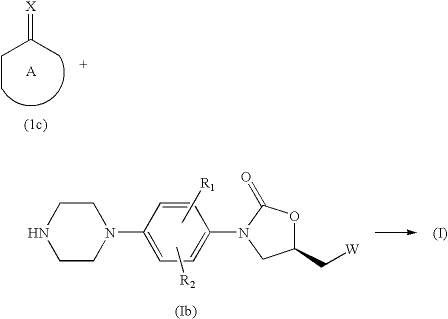
Route 3:

a) Compounds of general formula (Ie) may be obtained from compounds of general formula (Ia) by coupling with compounds of general formula (Id), employing different no sets of coupling agents depending upon the nature of (Ia) such as acid chlorides corresponding to (Ia), and bases such as Na₂CO₃, K₂CO₃ and the like; organic bases like triethylamine, pyridine, diisopropylethylamine and the like; Solvent such as acetone, THF may be used. Temperature in the range of −20° C. to reflux temperature of the solvent may be used. If (Ia) is an acid, suitable coupling agents like DCC, HOBT and the like may be used. Solvents such as dichloromethane, chloroform may be used.

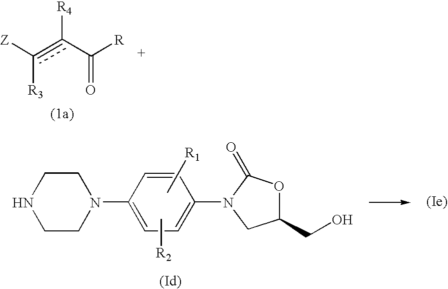

b) Compounds of general formula (If) may be obtained by treating the compounds of general formula (Ie), with appropriate sulfonyl chloride such as p-Ts-chloride, MsCl, benzene sulfonyl chloride and the like to get sulfonyl esters in presence of bases like triethylamine, pyridine, K₂CO₃ and the like or mixture thereof. Solvents such as DMF, DMSO, dichloromethane, dichloroethane, pyridine and the like and the mixtures thereof may be used. The temperature may range from 0° C. to reflux temperature of the solvent, preferably between 5° C. to 40° C.

Alternatively, the compounds of general formula (1f), where L is halide, may be obtained by treating the compounds of general formula (Ie) with SOCl₂, POCl₃, PCl₅, PBr₃ and the like, HBr/red P, in the presence of solvents such as DMF, DMSO, THF, benzene, CH₂Cl₂, dichloroethane and the like. The temperatures may range from 0° C. to 50° C. The mole ratio of halogenating agent to compounds (Ie) can range from 1:1 to 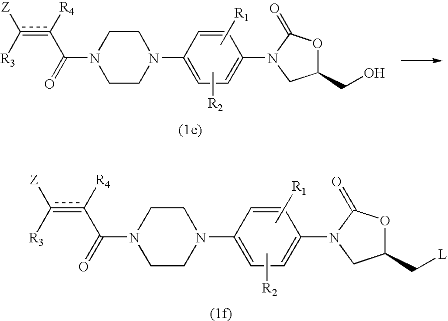
1:1.5.

c) Compounds of general formula (Ig) may be obtained by treating the compounds of general formula (If) with metal azides in solvents such as DMSO, pyridine, DMF and the like may be used. Temperature in the range of 10° C. to 120° C. may be used, preferably between 30° C. to 60° C.

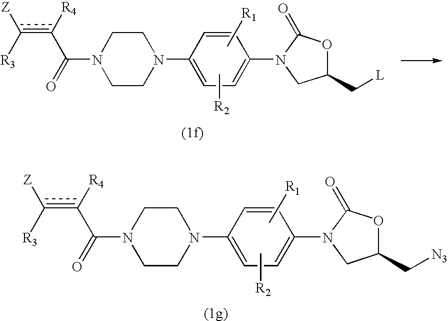

d). Compounds of general formula (Ih) can be obtained by (Ig) by use of triphenylphosphine and aquoues NH₃ or H₂O in solvents such as methanol, ethanol at temperatures between −10° C. to 30° C. The molar ratio of compounds (Ig) and reducing agent can range from 1:10 to 1:25.

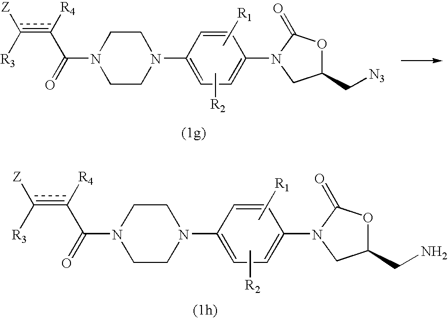

e) Compounds of general formula (Ii) can be obtained from compounds of general formula (Ih) by treating with carbon disulfide solution in presence of bases such as TEA & pyridine employing catalytic amount of esters of halogenated formic acid at temperatures between 0° C. and 50° C. depending upon the choice of bases.

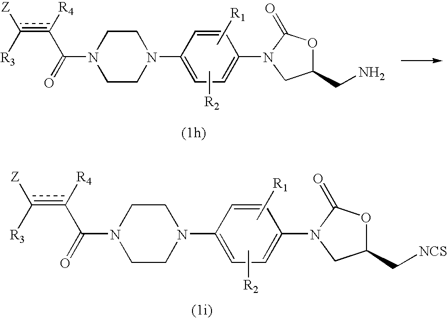

f) Compounds of general formula (Ij), where R₇ is NH₂, may be obtained from compounds of general formula (Ii) by treating it with ammonia in solvents such as methanol, ethanol and the like at temperatures ranging between −10° C. to 50° C.

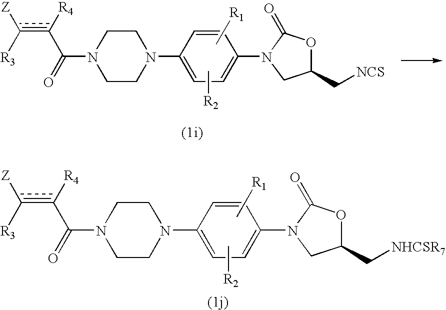

g) Alternatively, compound of general formula (Ik) may be obtained from compound of general formula (Ii) by treating it with solution of alkyl halides in solvents like ether or THF, at low temperature, preferably at 0-5° C.

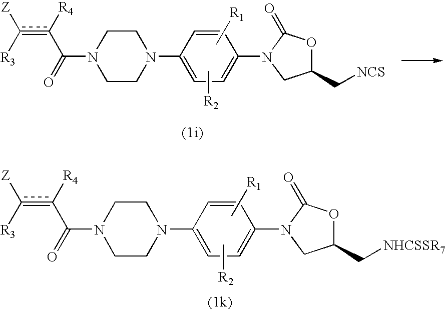

h) Alternatively, compound of general formula (Il) may be obtained from compound of general formula (Ii) by treating with metal hydrides such as sodium hydrides at low temperature in anhydrous alcohols as a solvent as well as a reactant. 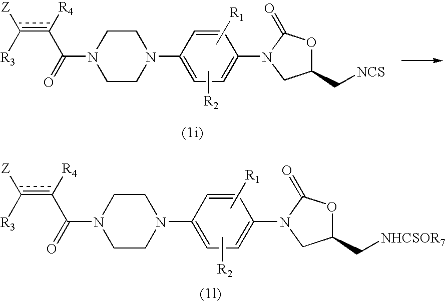
Route 4:

Compounds of general formula (Ib) when treated with compound of general formula (Im) in presence of metal carbonates such as K₂CO₃, Na₂CO₃, Cs₂CO₃ in solvents such as acetone, THF, at temperature ranging from 0-40° C. preferably at ca. 5° C., gives compound of general formula (1n). 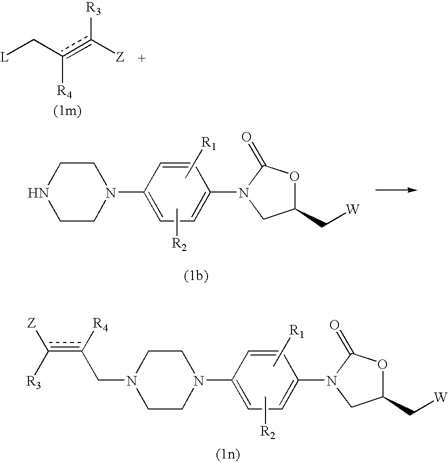
Route 5:

Compound of general formula (Ip) may be obtained from compounds of general formula (Io) by treating it with Lawesson's reagent in solvents such as THF, 1,4-dioxane, dichloromethane at temperature ranging from 30° C. to reflux temperature of the solvent 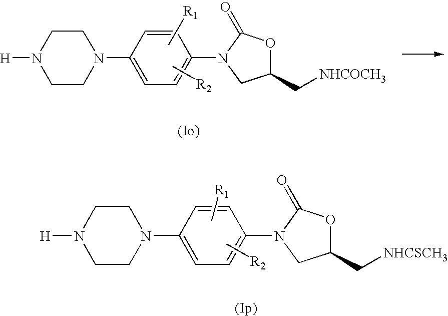
being used.

Pharmaceutically acceptable salts means salts formed by the addition of acids useful for administering the compounds of the present invention and includes hydrochloride, hydrobromide, sulfate, bisulfate, phosphate, acetate, propionate, lactate, mesylate, maleate, succinate, tartarate, citrate, 2-hydroxyalkylsulfonate, fumarate, oxalate, ascorbate and the like when a basic group is present in compound of formula (I).

These salts may be in hydrated form—some of the compounds of the invention may form metal salts such as sodium, potassium, calcium and magnesium salts and these are embraced by the term “pharmaceutically acceptable salts”.

It will be appreciated that in any of the above mentioned reactions any reactive group in the substrate molecule may be protected, according to conventional chemical practice. Suitable protecting groups in any of the above mentioned reactions are those used conventionally in the art. The methods of formation and removal of such protecting groups are those conventional methods appropriate to the molecule being protected. T. W. Greene and P. G. M. Wuts “Protective groups in Organic Synthesis”, John Wiley & Sons, Inc, 1999, 3^{rd} Ed., 201-245 along with references therein.

It will be appreciated that the above-mentioned preparation of the compounds of Formula (I), or a pharmaceutically acceptable salts thereof, and/or pharmaceutically acceptable solvates thereof employs (1d) or (1b) as a pure enantiomer to afford the compound of formula (I) as a single stereoisomer. Favorably, in a compound of formula (I) the preferred configuration at C-5 of the oxazolidinone ring of compounds claimed in the invention is (S)-under the Cahn-Ingold-Prelog nomenclature system. Since this (S)-enantiomer which is pharmacologically active. The racemic mixture is useful in the same way and for the same purpose as the pure (S)-enantiomers the difference lies in the fact that double as much racemic material will be required to produce the same antibacterial effect.

Because carbon-carbon double bond also exists in the compounds, the invention contemplates various geometric isomers and mixtures thereof resulting from the arrangement of substituents around these carbon-carbon double bonds. These substituents are designated as being in the E or Z configuration wherein the term “E” refers to higher order substituents on opposite sides of the carbon-carbon double bond, and the term “Z” refers to higher order substituents on the same side of the carbon-carbon double bond A thorough discussion of E and Z isomerism is provided in “Advanced Organic Chemistry. Reaction, Mechanisms, and Structure”, 4^{th} ed., John Wiley & Sons, New York, 1992, pp. 109-112.

Preferably the compounds of Formula (I), or a pharmaceutically acceptable salt thereof, and/or pharmaceutically acceptable solvate thereof is in optically pure form.

The absolute stereochemistry of the compounds may be determined using conventional methods, such as X-ray crystallography.

Another aspect of the present invention comprises, a pharmaceutical composition, containing at least one of the compounds of the general formula (I), their derivatives, their analogs, their tautomeric forms, their polymorphs, their prodrugs, their stereoisomers, their pharmaceutically acceptable salts, their pharmaceutically acceptable solvates thereof as an active ingredient, together with pharmaceutically employed carriers diluents and the like.

Pharmaceutical compositions containing a compound of the present invention may be prepared by conventional techniques, e.g. as described in Remington: the Science and Practice of Pharmacy, 19^{th} Ed., 1995. The compositions may be in the conventional forms, such as capsules, tablets, powders, solutions, suspensions, syrups, aerosols or topical applications. They may contain suitable solid or liquid carriers or in suitable sterile media to form injectable solutions or suspensions. The compositions may contain 0.5 to 20%, preferably 0.5 to 10% by weight of the active compound, the remaining being pharmaceutically acceptable carriers, excipients, diluents, solvents and the like.

The compounds of Formula I are useful in the treatment of microbial infections in humans and other warm blooded animals, by either oral, topical or parenteral administration.

Besides being useful for human treatment, these compounds are also useful for veterinary treatment of companion animals, exotic animals and farm animals including mammals, rodents, and the like. More preferred animals include horses, dogs and cats.

For the treatment of any of the above-mentioned diseases the compounds of formula (I) may be administered, for example, orally, topically, parenterally, in dosage unit formulations containing conventional non-toxic pharmaceutically acceptable carriers, adjuvants and vehicles.

The pharmaceutical composition is provided by employing conventional techniques. Preferably the composition is in unit dosage form containing an effective amount of the active component, that is, the compounds of formula I according to this invention.

The quantity of active component, that is, the compounds of formula I according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

In therapeutic use for treating bacterial infections in humans and animals that have been diagnosed with having bacterial infections, the compounds or pharmaceutical compositions thereof will be administered orally, parenterally and/or topically at a dosage to obtain and maintain a concentration, that is, an amount, or blood-level of active component in the animal undergoing treatment which will be antibacterially active. Generally, such antibacterially effective amount of dosage of active component will be in the range of about 0.1 to about 100 mg/kg, more preferably about 3.0 to about 50 mg/kg of body weight/day. However, it should be appreciated that the dosages may vary depending upon the requirements of the patient, the severity of the bacterial infection, and the particular compound being used. Also, it must be understood that the initial dosage administered may be increased beyond the upper level in order to rapidly achieve the desired blood level or the initial dosage may be smaller than the optimum and the and the daily dosage may be progressively increased during the course of treatment depending on the particular situation. If desired, the daily dose may also, be divided into multiple doses for administered, e.g. two to four times per day.

The compounds of the present invention may be administered alone or in combination with pharmaceutically acceptable carriers or diluents by any of the routes as previously indicated, in single or multiple doses. More specifically, the novel compounds described in the invention can be administered in a wide variety of different dosage forms, i.e., they may be combined with various pharmaceutically acceptable inert carriers in the form of tablets, capsules, lozenges, trochees, hard candies, powders, sprays, creams, salves, suppositories, jellies, gels, pastes, lotions, ointments, aqueous suspensions, injectable solutions, elixirs, syrups, and the like. The carriers may include solid diluents or fillers, sterile aqueous media and various nontoxic organic solvents etc. Moreover, for oral consumption, the pharmaceutical compositions can be suitably sweetened and/or flavored. In general, the therapeutically-effective compounds as described in the invention are present in the compositions at concentration levels ranging from 5% to 60% by weir, preferably 10% to 50% by weight.

For oral administration, the tablets may be combined with various excipients such as microcrystalline cellulose, sodium citrate, calcium carbonate, dipotassium phosphate and glycine along with various disintegrants such as starch more preferably corn, potato or tapioca starch, alginic acid, sodium carbonate and certain complex sillicates; together with binders like polyvinylpyrrolidone, sucrose, gelatin and acacia, humectants such as for example, glycerol; solution retarding agents, such as, for example paraffin; absorption accelerators such as, for example, quartenary ammonium compounds; wetting agents like cetyl alcohol and glycerol monostearate; absorbents like kaolin and bentonite clay. Additionally, magnesium stearate, sodium lauryl sulfate, talc, calcium stearate, solid polyethylene glycols and mixtures thereof are often added as lubricating agents for tabletting purposes. In the case of capsules, tablets and pills, the dosage form may also comprise buffering agents.

Similar type of solid compositions may also be employed as fillers and excipients in soft and hard gelatine capsules; preferred materials includes lactose, milk sugar or high molecular weight polyethylene glycols.

The active compounds can also be in micro-encapsulated form using one or more of the excipients noted above. The solid dosage forms of tablets, dragees, capsules, pills, and the granules can be prepared with coatings and shells such as enteric coatings, release controlling coatings and other coatings which are well known in the field of pharmaceutical formulation art. In such solid dosage forms the active compound may be admixed with at least one inert diluent such as sucrose, lactose and starch. They may also contain, additional substances for e.g. tableting lubricants and other substances like magnesium stearate and microcrystalline cellulose. In the case of capsules, tablets, and pills, the formulation may also contain buffering agents. They may also be so formulated that they release the active ingredient(s) only or preferentially in a certain part of the intestinal tract, optionally in a delayed manner. The same may be achieved using embedded agents like, for example, polymeric substances and waxes.

Liquid dosage forms for oral administration include pharmaceutically acceptable emulsions, microemulsions, solutions, suspensions, syrups, and elixirs. For such oral consumption it is desirable to combine the active ingredient with various sweetening or flavoring agents, coloring matter or dyes, if so desired. The diluents may be selected from water, ethanol, propylene glycol, isopropyl alcohol, ethyl carbonate, ethyl acetate, benzyl alcohol, benzyl benzoate, 1,3 butylene glycol, dimethyl formamide, oils for e.g. cottonseed, groundnut, corn, germ, olive, castor, sesame oils and the like, glycerol, tetrahydrofurfuryl alcohol, polyethylene glycols and esters of fatty acids like sorbitan and various combination thereof. For mammals other than humans, the composition of the active substance are suitably modified.

For parenteral administration, the solutions of the compound is prepared in either sesame or peanut oil or in aqueous propylene glycol. The aqueous solutions should be suitably buffered (preferably pH>8) if necessary, and the diluent should be first rendered isotonic. The aqueous solutions are suitable for intravenous injection purposes while the oily solutions are suitable for intra-articular, intramuscular and subcutaneous injection purposes. The aforesaid compositions can be readily prepared under sterile conditions following well known standard pharmaceutical techniques by persons skilled in the art.

For buccal administration the composition may take the form of tablets or lozenges formulated in conventional manner.

For transdermal and topical administration; the dosage forms will include ointments, pastes, creams, lotions, gels, powders, solutions, sprays and inhalants. Transdermal patches may be prepared following standard drug delivery techniques and applied to the skin of a mammal, preferably a human or a dog, to be treated. Ophthalmic solutions, ear drops, eye ointments, powders can also be used as a medium of providing therapeutic dosages to the patients as will be necessary.

The ointments, pastes, creams and gels may, in addition to the active ingredient, contain excipients like animal and vegetable fats, oils, waxes, paraffins, starch, tragacanth, cellulose derivatives, polyethylene glycols, silicones, bentonites, silicic acid, talc, zinc oxide or their mixtures.

Powders and sprays may contain, in addition to the active substance, excipients like lactose, talc, silicic acid, aluminium hydroxide, calcium silicates and polyamide powder, or their mixtures. Sprays will additionally contain propellants like chlorofluorohydrocarbons.

The pharmaceutically acceptable compounds of the present invention are useful antibacterial agents having a good spectrum of activity in vitro and against standard Gram-positive organisms, which are used to screen for activity against pathogenic bacteria. Notably, the pharmaceutically acceptable compounds of the present invention show activity against enterococci, pneumococci, and methicillin resistant strains of S.aureus and coagulase negative staphylococci, together with morganella strains. The antibacterial spectrum and potency of a particular compound may be determined in a standard test system. The activity is described in terms of the minimum inhibitory concentration (MIC) determined by microbroth dilution technique as per NCCLS standards.

Determination of Antibacterial Activity:

The minimum inhibitory concentrations (MIC's) of the compounds for the microorganisms listed in Table A were determined by preparing working solution for each compound of concentration of 128 μg/ml after dissolving it in DMSO. Two-fold serial dilution of the above solution was prepared in duplicates, using Mueller Hinton Broth, in 96 well Tissue culture plate with cover flat bottom wells to give a final volume of 150 μg/ml and concentration of compound ranging from 64 μg/ml-0.12 μg/ml. 30 μg/ml of Standard suspension of each organism which was prepared with turbidity equivalent to the 1:10 diluted 0.5 McFarland standard with density 10⁷ CFU/ml, was added to each well to get approximately a density of 10⁵ CFU/ml. These 96-well Tissue culture plate containing the test samples and positive and negative controls, were incubated at 37° C. for 16-18 hrs. The wells were visually inspected for growth and were also read at 630 nm by Automated Microplate Reader [(EL800) Trinity biotech.] and the MIC's were recorded as the lowest concentration of drug which inhibits the growth of bacteria. The compounds inhibited the growth of these bacteria with MIC's in a range of about 0.25 μg/ml to about 64 μg/ml.

Thus the compounds are useful for treating bacterial infections such as, but not limited to, those shown below in Table A.

TABLE A
Microorganism

Methicillin resistant Staphylococcus aureus
(ZYABL 006)
Staphylococcus epidermidis ATCC 12228
Enterococcus faecalis ATCC 29212
Staphylococcus aureus ATCC 33591
Staphylococcus aureus MTCC 737/ATCC 6538P

The invention is explained in detail by the examples given below, which are provided by way of illustration only and therefore should not be construed to limit the scope of the invention.

1H NMR spectral data given in the tables (vide infra) are are recorded using a 300 MHz spectrometer (Bruker AVANCE-300) and reported in δ scale. Until and otherwise mentioned the solvent used for NMR is CDCl₃ using Tetramethyl silane as the internal standard.

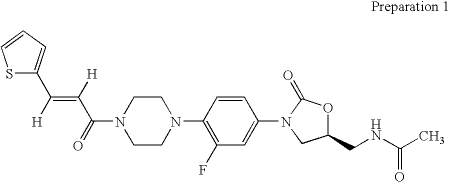

### (S)-N-[3-(3-Fluoro-4-{4-[3 thiophen-2-yl)-acryloyl]piperazinyl]-phenyl)-2-oxo-oxazolidin-5-yl-methyl]acetamide. (Compound No. 01)

To a solution of (S)-N-[[3-[3-fluoro-4-(N-piperazinyl)phenyl]-2-oxo-5-oxazolidinyl]methyl]acetamide (J. Med. Chem. 1996, 39, 673-679) (0.1 g) in chloroform (20 ml) was added. HOBt.H₂O (0.1 g), 1-(3-dimethyl aminopropyl)-3-ethylcarbodiimide hydrochloride (0.1 g) followed by 3-(2-thienyl)acrylic acid (0.045 g). The reaction mixture was stirred at ca 27° C. to which triethylamine (1 ml) was added. After stirring for 2 hrs. at ca 27° C. (TLC) the reaction mixture was diluted with CHCl₃ (30 ml.) and washed with DM water (50 ml). Organic layer was separated, dried over anhydrous sodium sulfate and concentrated under reduced pressure. The crude product was crystallized in EtOAc to afford the title compound as a white solid (75 mg, 53%) m.p. 223-225° C.

The following compounds were prepared following the above procedure.

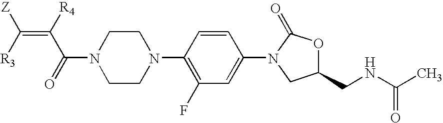

TABLE 1

| 1. | Z | R₄ | R₃₃ | Mol. Wt. | Yield |
|---|---|---|---|---|---|
| | 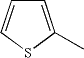 | H | H | 472 | 53% |
| | 7.8(1H d, J=15.06 Hz), 7.4(2H, dd, J=2.51 Hz), 7.3(2H, d, J=5.04 Hz), 7.2(1H, d, J=3.4 Hz), | | | | |
| | 7.0(2H, dd, J=3.48 Hz),), 6.9(t, J=9 Hz), 6.7(2H,d, J=15 Hz), 4.8(1H, m), 3.9(1H, t, J=9.0 Hz), | | | | |
| | 3.7(7H, m), 3.2(2H, m), 3.0(4H, t), 2.2(3H, s). | | | | |
| 2. | 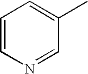 | H | H | Mol. Wt 467 | Yield 80% |
| | 8.7(1H, d, J=1.71 Hz), 8.5(1H, d, J=3.86 Hz) 8.1(1H, d, J=8.04 Hz), 7.6(2H, d, J=15.57 Hz), | | | | |
| | 7.5(2H, m), 7.2(1H, d, J=15.57 Hz), 7.1(1H, dd, J=1.86 Hz), 7.0(1H, t, J=9.12 Hz), 4.8(1H, t, | | | | |
| | J=9 Hz), 4.7(1H, m), 4.0(1H, t, J=9.0 Hz), 3.9(4H, t), 3.7(4H, m), 3.5(2H, d, J=4.95 Hz), | | | | |
| | 3.1(4H, t), 1.95(3H, s); (solvent used is CD₃OD + CDCl₃) | | | | |
| 3. | H | H | H | Mol. Wt | Yield |
| | | | | 390 | 20% |
| | 7.4(1H, dd, J=6.12 Hz), 7.0(2H, d, J=8.7 Hz), 6.9(1H; m), 6.0(1H, t), 4.7(1H, m), 4.0(1H, t), | | | | |
| | 3.7(5H, m), 3.0(8H, complex) 2.02(3H, s) | | | | |
| 4. | 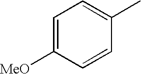 | H | H | Mol. Wt 496 | Yield 47% |
| | 7.6(1H, dd, J=15.33 Hz), 7.4(3H, m), 7.0(1H, dd, J=1.71 Hz), 6.9(1H, d, J=15.33 Hz), 6.18(1H, t, | | | | |
| | J=9.6 Hz), 6.7(1H, d, J=15.33 Hz), 6.1(1H, t), 4.8(1H, m), 4.0(1H, t), 3.7(10H, complex), 3.0(1H, t), | | | | |
| | 3.0(4H, t, J=4.53 Hz), 2.0(3H, s) | | | | |
| 5. | 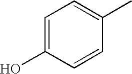 | H | H | Mol. Wt 482 | Yield 52% |
| | 7.6(1H, d, J=15.3 Hz), 7.3(4H, m), 6.9(1H, t, J=9.3 Hz), 6.8(2H, d, J=8.4 Hz), 6.7(2H, d, | | | | |
| | J=15.3 Hz), 4.7(1H, m), 3.0(4H, t), 2.0(3H, S), 4.1(2H, m), 3.8(4H, m), 3.6(4H complex) | | | | |
| | (solvent used is CD₃OD + CDCl₃) | | | | |
| 6. | 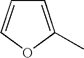 | H | H | Mol. Wt 456 | Yield 20% |
| | 7.5(3H, m), 7.0(1H, dd), 6.9(1H, t), 6.8(1H. d, J=15.0 Hz), 6.5(1H, d, 33.3 Hz), 6.4(1H, dd), | | | | |
| | 5.9(1H, t), 4.8(1H, m), 4.0(2H, t, J=8.97 Hz), 3.8(4H, d), 3.7(3H, complex), 3.0(4H, t), 2.0(3H, s). | | | | |
| 7. | 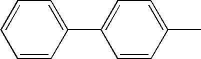 | H | H | Mol. Wt 542 | Yield 39% |
| | 7.6(1H, dd, J=12.99 Hz), 7.6(6H, m), 7.4(4H, m), 7.0(1H, dd), 6.9(1H, d, J=12.39 Hz), 5.9(1H, | | | | |
| | t), 4.7(1H, m), 4.0(1H, t, J=9 Hz), 3.8(4H, d), 3.6(3H, m), 3.1(4H, t), 2.0(3H, s). | | | | |
| 8. | Me | H | H | Mol. Wt | Yield |
| | | | | 404 | 72% |
| | 7.48(1H, dd, J=11.61 Hz, J=2.5 Hz), 7.0(2H, dd, J=8.76), 6.9(2H, m), 6.2(1H, dd, J=13.32 Hz), | | | | |
| | 1.65 Hz), 5.9(1H, t), 5.3(1H, m), 4.0(1H, t, J=8.9 Hz), 3.7(6H, m), 3.0(4H, t, J=5. Hz), 2.0(3H, s), | | | | |
| | 1.8(3H, q, J=1.56 Hz) | | | | |
| 9. | H | H | Me | Mol. Wt | Yield |
| | | | | 404 | 71% |
| | 7.4(1H, dd, J=2.55 Hz), 7.0(1H, dd, J=2 Hz), 6.51 Hz), 8.9(1H, t, J=9.0 Hz), 5.9(1H, t), 5.2(1H, t, | | | | |
| | J=9.0 Hz). 3.7(6H, m), 3.0(4H, s), 2.0(3H, s), 1.9(3H, s). | | | | |
| 10. | 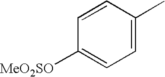 | H | H | Mol. Wt 560 | Yield 58% |
| | 7.6(1H, d, J=15.4 Hz). 7.5(1H, dd, J=11.85, Hz, 2.31 Hz), 7.4(1H, d, J=8.55 Hz), 2.31 Hz), 6.9(2H, | | | | |
| | m), 6.1(1H, t), 4.8(1H, m), 4.0(1H, t, J=9 Hz), 3.7(6H, m), 3.0(3H, S), 2.0(3H, s). | | | | |
| 11. | 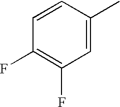 | H | H | Mol. Wt 502 | Yield 64% |
| | 7.6(1H, d, J=15.39 Hz), 7.5(1H, d, J=2.52 Hz, 11.61 Hz), 7.4(1H, d, J=2.52 Hz, 11.61 Hz), | | | | |
| | 7.3(4H, m), 7.0(2H, q, J=1.77 Hz), 6.9(1H, t, J=9.12 Hz), 6.8(1H, d, J=15.42 Hz), 4.7(1H, m), | | | | |
| | 4.0(1H, t, J=9 Hz), 3.9(4H, t), 3.6(3H, m), 3.3(4H, t), 2.0(3H, s) | | | | |
| 12. | 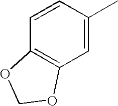 | H | H | Mol. Wt 510 | Yield 77% |
| | 7.8(1H, t), 7.6(1H, d, J=15.27 Hz), 7.0(3H, Hz), 6.7(1H, t), 6.0(2H, S), 4.7(1H, m), 4.0(1H, t, | | | | |
| | J=9.0 Hz), 3.8(4H, m), 3.5(2H, t), 3.0(4H, m), 1.9(3H, s) solvent used is | | | | |
| | CD₃OD + CDCl₃-d₆) | | | | |
| 13. | 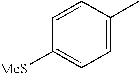 | H | H | Mol. Wt 512 | Yield 88% |
| | 7.6(1H, d, J=15.39 Hz), 7.4(3H, m), 7.2(2H, m), 7.0(1H, dd, J=1.92 Hz, 8.73 Hz), 6.9(1H, d, | | | | |
| | J=9.0 Hz), 6.8(1H, d, J=15.39 Hz), 6.0(1H, t), 3.8(1H, m), 4.0(1H, t, J=9 Hz), 3.5(6H, complex), 3.0 | | | | |
| | (4H, t, J=4.83 Hz), 2.5(3H, S), 2.0(3H, s) | | | | |
| 14. | 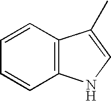 | H | H | Mol. Wt 505 | Yield 73% |
| | 7.9(1H, d, J=15.3 Hz), 7.5(2H, d), 7.45(2H, m), 7.40(1H, d, J=6.93 Hz), 7.0(1H, dd, J=2.04 Hz), | | | | |
| | 6.9(2H, d, J=9 Hz), 3.7(4H, t), 3.6(3H, m), 3.1(4H, t), 2.02(3H, s). | | | | |
| 15. | 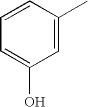 | H | H | Mol. Wt 482 | Yield 70% |
| | 7.6(1H, d, J=15.39 Hz), 7.5(1H, dd, J=2.55 Hz, 11.69 Hz), 7.1(1H, t, J=8.0 Hz), 6.8(4H, m), | | | | |
| | 4.7(1H, m), 4.0(1H, t, J=9 Hz), 3.6(6H complex), 3.0(4H, J=4.8 Hz), 2.0(3H, s)( (solvent used is | | | | |
| | CD₃OD + CDCl₃-d₆) | | | | |
| 16. | 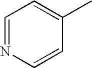 | H | H | Mol. Wt 467 | Yield 47% |
| | 8.6(2H, d, J=5.9 Hz), 8.2(1H, t, J=5.8 Hz), 7.6(2H, d, J=6.0 Hz), 7.5(3H, m), 7.1(1H, dd, | | | | |
| | J=6.6 Hz, 2.2 Hz), 7.0(t, 1H, J=9.0 Hz), 4.6(1H, m), 4.0(2H, t), 3.6(5H, complex), | | | | |
| | 2.9(4H, t), 1.8(3H, S). (solvent used is DMSO-d₆) | | | | |
| 17. | 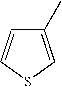 | H | H | Mol. Wt 472 | Yield 82% |
| | 7.7(1H, d, J=15.3 Hz), 7.5(4H, m), 7.2(1H, d), 6.9(1H, t, J=9.0 Hz), 6.7(d, J=15.27 Hz), 4.7(1H, m), | | | | |
| | 3.0(1H, t, J=9.0 Hz), 3.4(7H, m), 3.0(4H, t, J=4.82 Hz), 2.0(3H, s) (solvent used is | | | | |
| | CD₃OD + CDCl₃) | | | | |
| 18. | 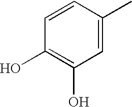 | H | H | Mol. Wt 498 | Yield 25% |
| | 7.7(1H, t), 7.5(1H, d, J=15.3 Hz), 7.4(1H, dd, J=2.5 Hz), 7.0(1H, dd, J=2 Hz), 6.9(3H, m), 6.8(1H, | | | | |
| | d, J=8.16 Hz), 6.7(1H, d, J=15.3 Hz), 4.7(1H, m), 4.0(1H, t, J=9 Hz), 3.6(5H, complex), 3.0(4H, s), | | | | |
| | 2.0(3H, s) (solvent used is CD₃OD + CDCl₃) | | | | |
| 19. | 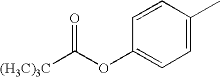 | H | H | Mol. Wt 566 | Yield 85% |
| | 7.5(1H, d, J=15 Hz), 7.5(2H, d, J=8.4 Hz), 7.49(1H, dd, J=11.64 Hz & 2.52 Hz), 7.1(4H, dd, J=8.4 | | | | |
| | Hz), 6.9(1H, t, J=9 Hz), 6.8(1H, d, J=14.9 Hz), 6.1(1H, t), 4.7(1H, m), 4(1H, t, J=9 Hz), 3.7(7H, m), | | | | |
| | 3.1(4H, t, J=1.77 Hz), 2(3H, s), 1.4(9H, s). | | | | |
| 20. | 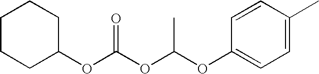 | H | H | Mol. Wt 653 | Yield 66% |
| | 7.7(1H, d, J=15.4 Hz); 7.5(2H, d, J=8.65 Hz); 7.4(1H, dd, J=11.73 & 2.5 Hz); 7.0(1H, dd, J=2 Hz); | | | | |
| | 6.8(2H, d, J=15.14 Hz); 5.9(1H, t); 4.7(2H, m); 4.0(1H, t, J=9); 3.7(7H, m); 3(4H, t); 1.98(5H, m); | | | | |
| | 1.7(2H, t); 1.3(4H, m). | | | | |
| 21. | 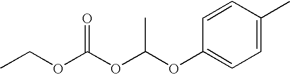 | H | H | Mol. Wt 598 | Yield 70% |
| | 7.6(1H, d, J=8.58 Hz); 7.5(2H, d, J=14.9 Hz); 7.4(1H, dd, J=11.64 & 2.5 Hz); 7.2(2H, d, J=15 Hz); | | | | |
| | 7.1(1H, dd J=6.9 & 1.8 Hz); 6.9(2H, m); 5.9(1H t); 4.7(1H, m); 4.35(3H, q); 4(1H, t, J=9 Hz); | | | | |
| | 3.7(3H, m); 3.1(4H, t, J=4.9 Hz); 2.01(3H, s); 1.4(3H, t, J=7 Hz). | | | | |
| 22. | 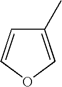 | H | H | Mol. Wt 456 | Yield 41% |
| | 8.24(1H, d, J=5.7 Hz) 8.03(1H, S), 7.7(1H, d), 7.51(1H, d, J=1.8 Hz), 7.13(1H, d, J=12.9 Hz), | | | | |
| | 7.43(1H, d, J=15.9 Hz), 7.04(t, 141, 4.6(1H, m), 4.07(1H, t), 3.69(4H, t), 2.97(4H, t), 3.8(2H, t), | | | | |
| | 3.38(2H, t), 1.81(3H, s) (solvent used is DMSO-d₆) | | | | |
| 23. | 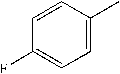 | H | H | Mol. Wt 484 | Yield 73% |
| | 7.6(1H, d, J=15.4 Hz); 7.5(2H, m); 7.4(1H, d, J=2.9 Hz); 7.0(4H, m); 6.9(1H, t, J=9.1 Hz); 6.8(1H, | | | | |
| | d, J=15 Hz); 4.7(1H, m); 4(1H, t, J=9 Hz); 3.7(5H, m); 3.6(2H, m); 3.1(4H, t, J=1.77 Hz); 2(3H, s). | | | | |
| 24. | 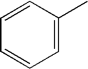 | H | H | Mol. Wt 466 | Yield 80% |
| | 7.6(1H, d, J=15.4 Hz), 7.5(2H, m), 7.4(1H, dd, J=2.52 Hz), 7.38(3H, m), 7.0(1H, dd, J=1.86 Hz), | | | | |
| | 6.9(1H, m), 6.0(1H, t), 4.8(1H, m), 4.0(1H, t, J=8.94 Hz), 3.9(4H, d), 3.6(3H, m), 3.0(4H, t, | | | | |
| | J=4.6 Hz), 2.0(3H, s). | | | | |
| 25. | 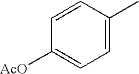 | H | H | Mol. Wt 524 | Yield 57% |
| | 7.65(1H, d, J=15.4 Hz), 7.5(2H, d, J=8.7 Hz), 7.4(1H, dd, J=2.4 & 11.7 Hz), 7.1(2H, d, J=8.7 Hz); | | | | |
| | 7.0(1H, d, J=2.1 Hz); 6.9(1H, d, J=9 Hz); 6.8(1H, d, J=15 Hz), 6.3(1H, t, J=6.3 Hz); | | | | |
| | 4.7(m, 1H); 4(1H, t, J =9 Hz); 3.8(4H, m); 3.09(4H m); 2.3(3H, s); 2.0(3H, s). | | | | |
| 26. | 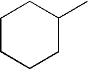 | H | H | Mol. Wt 472 | Yield 60% |
| | 7.49(1H, dd, J=11.64 Hz, 2.46 Hz), 7.09(1H, dd, J=1.65 Hz, 8.73 Hz), 6.8(2H, m), 6.2(1H, dd, | | | | |
| | J=14.1 Hz), 4.79(1H, m), 4.02(1H, t), 3.9(1H, t, J=8.9 Hz), 3.7(7H, m), 3.0(4H, t, J=4.7 Hz), | | | | |
| | 2.02(3H, s), 2.1(1H, m), 1.7(4H, m), 1.19(6H, m). | | | | |
| 27. | 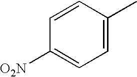 | H | H | Mol. Wt 511 | Yield 55% |
| | 8.2(2H, d, J=8.76 Hz), 7.7(3H, m), 7.5(2H, m), 7.12(2H, m), 6.9(1H, t, J=9.1 Hz), 4.8(1H, m), | | | | |
| | 4.0(1H, t, J=9 Hz), 3.8(5H, m), 3.6(2H, t, J=5.5 Hz), 3.1(4H, m), 1.9(2H, s). (solvent used is | | | | |
| | CDCl₃ + DMSO-d₆) | | | | |
| 28. | 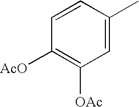 | H | H | Mol. Wt 582 | Yield 81% |
| | 7.6(1H, d, J=15.3 Hz), 7.45(1H, dd, J=2.7 & 11.7 Hz), 7.38(1H, d), 7.2(1H, d, J=8.1 Hz), | | | | |
| | 7.09(dd, J=2 & 6.3 Hz), 6.95(1H, t, J=9 Hz), 6.8(1H, d, J=15.3 Hz), 6.1(1H, t), 4.76(1H, m), | | | | |
| | 4.04(1H, t), 3.75-3.5(3H, complex), 3.07(5H, t), 2.3(7H,s), 2(4H, s), 1.65(2H, s). | | | | |
| 29. | 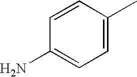 | H | H | Mol. Wt 481 | Yield 6.9% |
| | 7.52-7.46(2H, dd, J=2.4, 2.7 Hz); 7.39-7.34(1H, d, J=15 Hz), 718-7.15(1H, dd, J=2.4, 2.1 Hz), | | | | |
| | 6.93-6.8(1H, d, J=15 Hz), 6.55-6.52(2H, d, J=8.4 Hz), 4.69-4.6(1H, m), | | | | |
| | 4.10-4.04(1H, t, J=9 Hz), 4-3.66(4H, t), 2.95(4H, b), 1.81(3H, s) (solvent used is DMSO-d₆) | | | | |
| 30. | 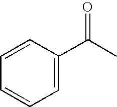 | H | H | Mol. Wt 494 | Yield 40% |
| | 8.22(1H, t), 8.04-8.01(2H, t, J=7.2 Hz), 7.81-7.76(1H, d, J=15.3 Hz), 7.59-7.54(2H, t, J=7.2 Hz), | | | | |
| | 7.51-7.46(2H, dd, J=2.4 Hz), 7.50-7.45(1H, d, J=15.3 Hz), 7.16-7.15(1H, d, J=2.1 Hz), | | | | |
| | 4.69(1H, m), 4.10-4.04(1H, t, J=8.7, 9 Hz), 3.71(4H, b), 3.0(4H, b), 1.81(3H, s). | | | | |
| | (solvent used is DMSO-d₆) | | | | |
| 31. | 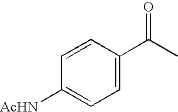 | H | H | Mol. Wt 551 | Yield 12% |
| | 8.03(1H, d); 7.81(1H, d); 7.52(1H, d); 7.47(1H, d); 7.19(1H, d); 7.1(1H, t); 4.71(1H, m); 4.1(1H, t); | | | | |
| | 3.73(4H, m); 3.23(4H, m); 2.02(3H, s); 1.82(3H, s). (solvent used is DMSO-d₆) | | | | |
| 32. | 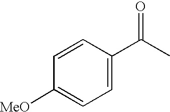 | H | H | Mol. Wt 524 | Yield 13% |
| | 8.06(2H, d, J=8.7 Hz), 8.03(1H, d, J=14 Hz), 7.53(1H, d, J=14 Hz), 7.5(1H, dd, J=2.4 & 10 Hz), | | | | |
| | 7.1(1H, dd, J=2 & 6 Hz), 6.98(2H, d, J=9.2 Hz). 6.92(1H, t, J=9.2 Hz), 5.9(1H, t, J=6 Hz), | | | | |
| | 4.7(1H, m), 4.02(1H, t, J=9 Hz), 3.81(3H, t), 3.1(5H, t), 2.1(4H, t), 1.52(6H, s) | | | | |
| 33. | 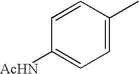 | H | H | Mol. Wt 523 | Yield 27% |
| | 7.60-7.65(1H, d, J=15.6 Hz), 7.46-7.52(3H, complex), 7.06-7.10(1H, dd, J=2.1, 6.6 Hz), | | | | |
| | 6.83-6.88(1H, d, J=15.3 Hz), 4.7(1H, m), 3.98-4.0(1H, t, J=9 Hz), 3.08(4H, s), 2.1(3H, s), | | | | |
| | 1.98(3H, s). (solvent used is CDCl₃ + DMSO-d₆) | | | | |
| 34. | 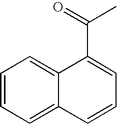 | H | H | Mol. Wt 544 | Yield 21% |
| | (1H, d, J=8.1 Hz), 8.1(1H, d, J=8.4 Hz), 7.9(1H, d, J=8.1 Hz) 7.72(1H, ddd, J=7.2 Hz), 7.62(1H, | | | | |
| | dd, J=6.9 Hz), 7.52(1H, d, J=8.1 Hz), 7.5(1H, dd, J=2 & 9.8 Hz), 3.9(4H, b.s.), 3.7(9.1 H,), | | | | |
| | 3.6(1H, complex), 3.39(2H, s), 3.08(2H, b), 2.6(6H, s) 7.1(1H, dd, J=2 Hz & 7.2 Hz), 7(1H, t), | | | | |
| | 4.7(1H, m); 4.6(9.1 H), 4.05(1H, t), 2.01(3H, s) | | | | |
| 35. | 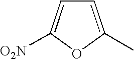 | H | H | Mol. Wt 501 | Yield 40% |
| | 7.5(1H, d, J=15.3 Hz), 7.4(1H, d, J=14.1 Hz); 7.36(1H, d, J=3.73 Hz), 7.10(1H, d, J=8.7 Hz), | | | | |
| | 6.9(1H, t, J=9 Hz), 6.7(1H, d, J=3.6 Hz) 4.7(1H, m) (1H, J=9 Hz); 3.7(4H, m); 3.1(4H, m); | | | | |
| | 2.02(3H, s). | | | | |
| 36. | 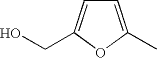 | H | H | Mol. Wt 486 | Yield 41% |
| | 7.48(1H, dd, J=17.04 Hz), 7.42(1H, d, J=15.05 Hz); 7.08(1H, dd, J=9.1 Hz); 6.93(1H, t, J=6.02 Hz); | | | | |
| | 6.84(1H, d, J=15 Hz); 6.51(1H, d); 6.35(1H, d, J=9.1 Hz); 4.76(1H, m); 4.64(2H, s); | | | | |
| | 4.05(1H, t, J=6 Hz); 3.88(2H, m); 3.82(1H, m); 3.76(4H, m); 3.06(4H, m). | | | | |
| 37. | 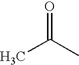 | H | H | Mol. Wt 432 | Yield 33% |
| | 7.49(1H, dd, J =2 Hz & 11.6 Hz), 7.2(1H, d, J=16 Hz), 7.1(1H, dd, J=2 & 7.5 Hz), 6.9(1H, t, | | | | |
| | J=9.06 Hz), 4.78(1H, m), 4.05(1H, t), 3.7(3H, t), 7.06(1H, d, J=15.36 Hz), 6.3, (1H, t) | | | | |
| | 3.89(2H, t), 3.6(2H, complex) (solvent used is DMSO-d₆) | | | | |
| 38. | 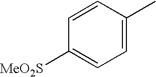 | H | H | Mol. Wt 544 | Yield 31% |
| | 8.01(2H, d, J=8.51 Hz), 7.94(2H, d, J=8.48 Hz), 7.61(1H, d, J=15.43 Hz), 7.52(1H, d, J=15.43 Hz), | | | | |
| | 7.47(1H, dd, J=17.31 Hz), 7.19(1H, d, J=11.16 Hz), 7.11(1H, t, J=6.15 Hz) 4.72(1H, m). | | | | |
| | 4.10(1H, t, J=5.98 Hz), 3.88(2H, m), 3.73(4H, m), 3.40(2H, m), 3.31(3H, s) 3.0(4H, m), 2.02(3H, s). | | | | |
| | (solvent used is DMSO-d₆) | | | | |
| 39. | 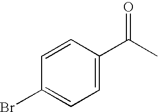 | H | H | Mol. Wt 572 | Yield 45% |
| | 7.9(3H t), 7.6(2H, d, J=8.58 Hz), 7.5(1H, d, J=14.9 Hz), 4(1H dd, J=11.64, 2.52 Hz). 7.0(1H, dd, | | | | |
| | J=6.9, 1.8 Hz), 6.9(1H t, J=9 Hz), 5.9(1H, t), 4.7(1H, m), 4.0(1H t, J=9 Hz), 3.9(2H, t). | | | | |
| | 3.8(2H, t), 3.6(3H, m), 3.0(4H, t, J=4.9 Hz), 2.0(3H, s). | | | | |
| 40 | 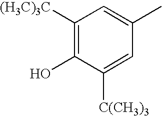 | H | H | Mol. Wt 594 | Yield 40% |
| | 7.6(1H, d, J=15.3 Hz), 7.4(1H, dd, J=2.4 & 11.7 Hz), 7.3(3H, s), 7(1H, dd, J=2.1 & 6.9 Hz), | | | | |
| | 6.8(1H, t, J=9 Hz), 6.7(1H, d, J=15, 5.4 Hz), (1H, s), 4.7(1H, m), 3.9 (1H, t, J=10.2 Hz); | | | | |
| | 3.8(4H, br), 3.6-3.7(3H, complex), 3(4H, br), 2(3H, s), 1.46(18H, s). | | | | |
| 41. | 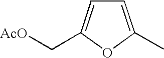 | H | H | Mol. Wt 528 | Yield 55% |
| | 7.5(2H, t, J=2.55 Hz), 7.4(1H, t, 2.55 Hz), 7.0(1H, dd, 1.86, 6.9 Hz), 6.9(1H, t, J=9.0 Hz), | | | | |
| | 6.8(1H, d, J=15 Hz), 6.4(2H, dd, J=3.3 Hz), 6.0(1H, m), 5.0(2H, s), 4.7(1H, m), 2.1(3H, s), | | | | |
| | 2.0(3H, s). | | | | |
| 42. | 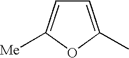 | H | H | Mol. Wt 470 | Yield 33% |
| | 7.48(1H, dd, J=2.52 Hz), 7.45(2H, d, J=15 Hz), 7.0(1H, d, J=3.18 Hz), 6.9(1H, t, J=9.1 Hz), | | | | |
| | 6.7(1H, d, J=14.9 Hz), 6.4(1H, d, J=3.18 Hz), 6.0(2H, q), 4.7(1H, m), 4.0(1H, t, J=9 Hz), | | | | |
| | 3.8(4H, s), 3.6(3H, m), 3.0(4H, t), 2.3(3H, s), 2.0(3H, s). | | | | |
| 43. | 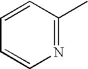 | H | H | Mol. Wt 467 | Yield 54% |
| | 8.6(1H, d, J=4 Hz), 7.6(3H, m), 7.3(1H, d, J=7.7 Hz), 6.9(1H, t, J=9 Hz), 7.0(1H, dd, J=2, | | | | |
| | 11.6 Hz), 7.4(1H, dd, J=2.52, 11.6 Hz), 6.0(1H, t), 4.7(1H, m), 4.0(1H, t, J=9 Hz), 3.9(4H, | | | | |
| | t, J=6.78 Hz), 3.7(3H, m), 3.0(4H, t, J=5 Hz), 2.0(3H s). (solvent used is DMSO-d₆) | | | | |
| 44. | 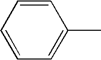 | — | — | Mol. Wt. 464 | Yield 57.97% |
| | 7.5(2H, t, J=6.57 Hz), 7.4(3H, m), 7.0(1H, dd, J=1.9, 8.7 Hz), 6.9(1H, t), 6.5(1H, t) 4.7(2H, m), | | | | |
| | 4.0(3H, t, J=4.4 Hz), 3.8(2H, t J=5.0 Hz), 3.6(3H, m), 2.0(3H, s) | | | | |
| 45. | H | H | 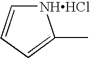 | Mol. Wt. 455 | Yield 80% |
| | 7.49(1H, dd, J=16.74 Hz), 7.06(1H, dd, J=10.1 Hz), 6.95(1H, t, J=6.0 Hz), 6.91(1H, m), 6.75(1H, d, | | | | |
| | J=12.6 Hz), 6.44(1H, m), 6.23(1H, m), 5.84(1H, d, J=12.57 Hz), 4.9(1H, m), 4.02(1H, t, J=6.1 | | | | |
| | Hz), 3.80(4H, m), 3.75(2H, m), 3.62(1H, m), 3.07(4H, m), 2.02(3H, s) | | | | |
| 46. | 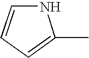 | H | H | Mol. Wt. 455 | Yield 90% |
| | 11.3(br, 1H), 8.24(1H, t, J=11.43 Hz), 7.52(1H, dd), 7.38(1H, d, J=15.21 Hz), 7.1(1H, dd, | | | | |
| | J=2, 9.3 Hz), 6.9(1H, d, J=15.21 Hz), 6.1(1H, br), 4.7(1H, m), 4.0(1H, t, J=9.15, 9 Hz), 3.7(4H, m), | | | | |
| | 3.31(4H, m), 1.8(3H, m) (solvent used is DMSO-d₆) | | | | |
| 47. | —COOH | H | H | Mol. Wt. | Yield |
| | | | | 434 | 38% |
| | 12.9(br, 1H), 8.23(1H, t), .51(1H, dd, J=14.32, 2.37 Hz), 7.47(1H, dd), 7.43(1H, d, | | | | |
| | J=15.39 Hz), 7.09(1H, t), 6.54(1H, d, J=15.36 Hz), 4.71(1H, m), 4.10(1H, t), 3.69(bs, 4H), | | | | |
| | 3.4(4H, m), 1.81(3H, s). solvent used is DMSO-d₆) | | | | |
| 48. | 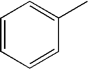 | CN | H | Mol. Wt. 491 | Yield 32% |
| | 7.8(1H, dd, J=2.1, 1.2 Hz), 7.4(4H, m), 7.0(1H, dd, J=1.8 Hz), 4.0(1H, t), 3.8(4H, m), | | | | |
| | 3.6(3H, m), 3.1(4H,br, 2.02(3H, s). | | | | |
| 49. | 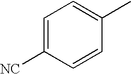 | H | H | Mol. Wt. 491 | Yield 60% |
| | 8.22(1H, br), 7.85(2H, d, J=8.46 Hz), 7.51(1H, dd, J=17.19 Hz), 7.40(1H, d, J=15.27 Hz), | | | | |
| | 7.46(2H, d, J=8.46), 7.18(1H, dd, 11.1), 7.10(1H, d, J=15.27), 7.05(1H, t, J=6.15), 4.69(1H, m), | | | | |
| | 4.04(1H, t, J=5.99), 3.71(4H, M), 3.68(1H, m), 3.40(2H, m), 2.99(4H, m), 1.81(3H, s). | | | | |
| | (solvent used is DMSO-d₆) | | | | |
| 50. | 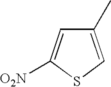 | H | H | Mol. Wt. 517 | Yield 22% |
| | 8.0(1H, d, J=4.3), 7.7(1H, d, J=15.2), 7.5(1H, dd, J=11.7, 2.5), 6.9(2H, m), 5.9(1H, m), | | | | |
| | 4.7(1H, m), 3.9(1H, t, J=9), 3.8(2H, s), 3.7(5H, m), 3.1(4H, s,), 2.0(3H, s). | | | | |
| 51. | 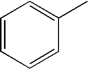 | CO₂Me | H | Mol. Wt. 524 | Yield 70% |
| | 7.76(1H, s), 7.55(1H, dd, J=16.3), 7.39(5H, m), 7.26(1H, dd, J=10), 6.77(1H, t, J=5.98), | | | | |
| | 6.15(1H, m), 4.02(1H, t, J=5.98), 3.99(1H, m), 3.93(4H, m), 3:39(4H, m), 3.07(2H, m), | | | | |
| | 2.96(3H, s), 2.04(3H, s). | | | | |
| 52. | 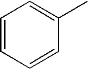 | COOH | H | Mol. Wt. 510 | Yield 60% |
| | 8.22(1H, bs), 2.99(4H, m), 7.5(1H, s), 7.48(1H, J=16.3), 7.39(5H, m), 7.13(1H, dd,. J=10), | | | | |
| | 6.92(1H, t, J=5.98), 4.81(1H, m), 4.04(1H, t, J=5.98), 3.99(1H, m), 3.83(4H, m), 3.39(4H, m), | | | | |
| | 3.07(2H, m), 2.043H, s). (solvent used is DMSO-d₆) | | | | |
| 53. | 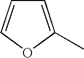 | COOMe | H | Mol. Wt. 514 | Yield 35% |
| | 7.53(1H, s), 7.5(1H, d, J=3.5), 7.46(1H, dd, J=16.27); 7.06(1H, dd, J=11.21), 6.92(1H, | | | | |
| | t, J=6.05), 6.76(1H, t, J=6.05), 6.51(1H, m), 6.04(1H, bs), 4.07(1H, m), 4.04(1H, t, J=5.98), | | | | |
| | 3.77(4H, m), 3.71(3H, m), 3.62(1H, m), 3.60(2H, m), 3.12(4H, m), 2.01(3H, s). | | | | |
| 54. | 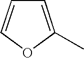 | COOH | H | Mol. Wt. 500 | Yield 31% |
| | 8.26(1H, br), 7.81(1H, s), 7.5(1H, dd, J=17.16), 7.22(1H, m), 7.16(1H, dd, J=17.16), | | | | |
| | 7.06(1H, dd, J=11.5), 6.76(1H, d, J=3.48), 6.59(1H, m), 6.04(1H, bs), | | | | |
| | 4.68(1H, m), 4.05(1H, t, J=5.59), 3.77(4H, m), 3.62(1H, m), 3.39(2H, m), 3.0(4H, m). | | | | |
| | (solvent used is DMSO-d₆) | | | | |
| 55. | 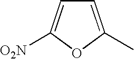 | COOMe | H | Mol. Wt. 559 | Yield 47% |
| | 8.24(bs, 1H), 7.76(1H, d, J=3.87), 7.45(1H, dd, J=17.25), 7.29(1H, dd, J=3.94), | | | | |
| | 7.17(1H, dd, J=11.09), 7.0491H, t, J=6.19), 4.71(1H, m), 4.09(1H, t, J=5.99), | | | | |
| | 3.79(4H, m), 3.65(1H, m), 3.44(2H, m), 3.32(3H, s), 2.86(4H, m), 1.81(3H, s). | | | | |
| | (solvent used is DMSO-d₆) | | | | |
| 56. | 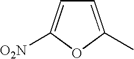 | COOH | H | Mol. Wt. 545 | Yield 62% |

8.98(1H, bs), 8.23(1H, s), 7.76(1H, d, J=3.87), 7.45(1H, dd, J=17.17), 7.22(1H, d, J=3.94),
7.17(1H, dd, J=11.09), 7.04(1H, t, J=8.19), 4.71(1H, m), 4.99(1H, t, J=5.99), 3.79(4H, m),
3.65(1H, m), 3.44(2H, m), 2.86(4H, m), 1.81(3H, s). (solvent used is DMSO-d₆)

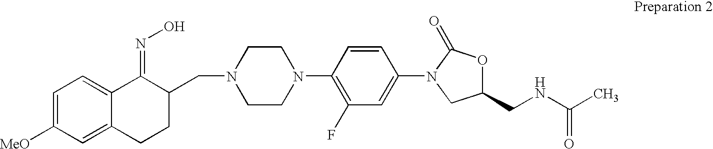

### (S)-N-(3-{3-Fluoro-4-[4-(6-methoxy-1-oxo-1,2,3,4-tetrahydronapthalen-2-yl methyl)-piperazin-1-yl]-phenyl]-2-oxo-oxazolidin-5-yl methyl)acetamide (Compound No. 61)

A cold solution of (S)-N-[[3-[3-fluoro-4-(N-1-piperazinyl)-phenyl]-2-oxo-5-oxazolidinyl]methyl acetamide (0.17 g) in methanol (5 ml) was added gradually to a stirred, cold solution of 37% aq. Formaldehyde (2 ml) in methanol (5 ml). The reaction mixture was kept in a freezing mixture of ice-salt (−10° C. to −15° C.) for 1 hour. The solvents were removed in vacuum and the residue was dissolved in methanol (5 ml). The resulting solution was cooled in a freezing mixture and a solution of dry HCl (g) in diethyl ether was added. The solvents were removed in vacuum and a solution of 6-methoxy-α-tetralone (0.039 g) in methanol (2 ml) was added to the resulting mass. The reaction mixture was heated on a water bath for 15-20 minutes. The solid separated was filtered to afford a sticky solid which was chromatographed on silica gel with 0-3% MeOH/CHCl₃ gradient to give the title compound as a white solid (50 mg, 18%).

The following compounds were prepared following a similar procedure as described above:

TABLE 2

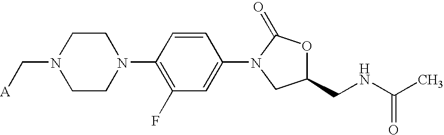

| 57. | A | Mol. Wt. | Yield (%) |
|---|---|---|---|
| | 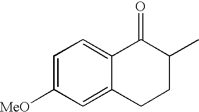 | Mol. Wt. 524.58 | Yield 29% |
| | 7.98(1H, d, J-8.8 Hz), 7.61(1H, d, J =10.8 Hz), 6.85(1H; d, | | |
| | J=7.59 Hz), 4.78(1H, m); 3.8(3H, s), 2.04(3H, s), 6.03(1H, m); | | |
| | 3.0(4H, broad d); 3.4(4H, complex); 7,(2H, bs); 1.23(4H, | | |
| | complex). 1.5(16H, br) | | |
| 58. | 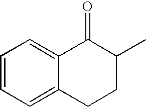 | Mol. Wt 494.56 | Yield 20% |
| | 7.95(1H; dd, J=1.5 & 6.8 Hz), 7.5(2H, m), 7.32(2H, t), | | |
| | 7.14(1H; dd, J =2.1 & 8 Hz); 7.08(1H; t, 9=9.1 Hz), 4.8(2H, m), | | |
| | 4.1(1H, t), 3.78(1H, quart), 3.55(2H, d), 2.6(3H, m), 2.6(3H, m), | | |
| | 3.08(7H, m), 1.9(4H, complex), 4.6(1H, s) (solvent used is | | |
| | CD₃OD) | | |
| 59. | 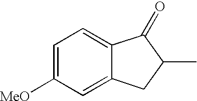 | Mol. Wt 510.56 | Yield 20% |
| | 7.69(1H; dd, J=7.89 Hz), 7.54(1H; d, J =12.6H); 6.94(1H; d, | | |
| | J=7.8 Hz), 6.43(1H, m), 4.8(1H, m), 3.8(3H, 3), 3.4(7H, | | |
| | complex), 3.3(8H, complex), 7.0(3H, t), 1.73(3H, bs), 4.02(2H, s) | | |
| 60. | 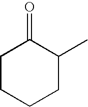 | Mol. Wt 446.5 | Yield 22% |
| | 7.5(1H, d), 6.9(2H, t), 4.7(1H, m), 2.01(3H, s), 3.4(2H, t), | | |
| | 2.03(3H, s), 4.03(1H, t), 3.8(complex), 3.37(4H, complex), | | |
| | 3.14(1H, bs,), 2.5(2H, bs), 1.37(6H, t) | | |
| 61. | 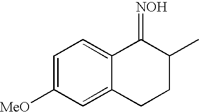 | Mol. Wt 540 | Yield 59% |
| | 7.44(1H; dd, J=2.4 & 11.6 Hz), 7.07(1H, dd, J=2.1 & 6.78 Hz), | | |
| | 6.95(1H; t, J=9.09 H.z), 4.84(1H, m), 3.35(4H, t), 3.10(4H, t), | | |
| | (3H, s), 3.7(5H, m), 3.71(2H, s), 1.5(2H, complex) | | |
| | (the solvent used is CDCl₃ ÷ CD₃OD) | | |
| 62. | 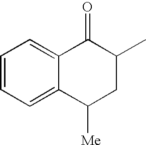 | Mol. Wt 508.5 | Yield 12% |

8.02(1H, d J=8.76 Hz), 7.75(1H; t, J=2.5 & 11.6 Hz),
7.04(1H; dd, J=2.2 & 6.8 Hz), 6.9(1H; t, J=9.12 Hz); 6.84(1H; dd,
J=2.49 & 6.27 Hz); 6.7(1H. d, J=2.37 Hz), 4.8(1H, m), 4.3(1H,
dd), 2.37(1H, m)

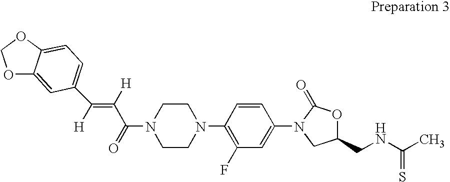

### (S)-N-[3-{4-(4-(3-Benzo[1,3]-dioxol-5-yl acryloyl)-piperazin-1-yl]-3-fluorophenyl]-2-oxo-oxazolidin-5-yl methyl]thioacetamide (Compound No. 63)

A stirred suspension of (S)-N-[[3-Fluoro-4-(N-1-piperazinyl)-phenyl]-2-oxo5-oxazolidinyl]methyl acetamide (0.2 g) in toluene (25 ml) was treated with Lawesson's reagent (0.24 g) under nitrogen atmosphere and refluxed for 5 hrs (TLC). The solvents were evaporated and the residue was chromatographed on silica gel using eluent 0-1% methanolic ammonia/CH₂Cl₂. The resulting solution was concentrated and was taken as such for reaction.

(S)-N-[[3-Fluoro-4-(N-1-piperazinyl)-phenyl]-2-oxo5-oxazolidinyl]methyl thioacetamide prepared as above (0.2 g) was taken in dichlormethane (50 ml). To this solution was added HOBt.H₂O (0.2 g) and 1-(3-Dimethylaminopropyl)-3-ethylcarbodiimide hydrochloride (0.2 g) followed by 3,4-methylene dioxycinnamic acid (0.109 g). The reaction mixture was stirred at ca 27° C. to which triethylamine (1 ml) was added. The reaction mixture was stirred for 3 hrs at 27° C. [TLC]. The reaction mixture was washed with DM water, organic layer was separated and dried over anhydrous sodium sulfate and solvents were evaporated. The resulting residue was chromatographed over silica gel with mobile phase 0-5% methanol/CH₂Cl₂. The resulting solution was concentrated to afford the title compound (0.1 g, 33%).

The following compounds were prepared according to the above procedure.

TABLE 3

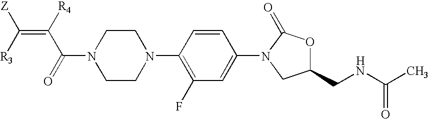

| 63. | Z | R₄ | R₃ | Mol. Wt. | Yield |
|---|---|---|---|---|---|
| | 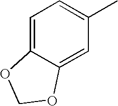 | H | H | 526 | 31% |
| | 7.41-7.46(1H, d, J=15.6 Hz), 7.05-7.19(4H, complex), 6.09(1H, | | | | |
| | d), 6.05(2H, s), 4.9(1H, m), 4.09-4.15(1H, t, J=9.0, 9.3 Hz), | | | | |
| | 3.8(4H, b), 2.97(4H, b), 2.42(3H, s) (the solvent used is | | | | |
| | DMSO-d₆) | | | | |
| 64. | 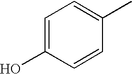 | H | H | Mol. Wt 498 | Yield 78% |
| | 7.6(1H, d, J=15.33 Hz), 7.4(3H, t), 7.1(1H, d, J=8.82 Hz), | | | | |
| | 6.9(1H, t, J=9.0 Hz). 6.8(2H, d, J=8.52 Hz), 6.7(1H, d, J=15.36 Hz), | | | | |
| | 4.9(1H, 2), 4.1(3H, m), 3.1(s, 4H), 2.5(s, 3H). (the solvent used is | | | | |
| | CDCl₃ + drops of CD₃OD) | | | | |
| 65. | 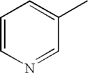 | H | H | Mol. Wt 483 | Yield 43% |
| | 8.7(1H, d, J=1.74 Hz), 8.5(2H, 2, J=1.44 Hz), 7.8(1H, d), | | | | |
| | 7.7(1H, d, J=15.54 Hz), 7.5(1H, q, J=2.5 m, 11.73 Hz), 7.0(2H, m), | | | | |
| | 5.0(1H, m), 3.8(8H, m), 3.1(4H, t), 2.5(8H, 2). (the solvent used is | | | | |
| | CDCl₃ + drops of CD₃OD) | | | | |
| 66. | 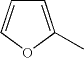 | H | H | Mol. Wt 472 | Yield 62% |
| | 7.7(1H, 3), 7.4(2H, m), 7.0(1H, dd, J=2.22 Hz), 6.9(1H, t), | | | | |
| | 6.8(1H, d, J=15.0 Hz), 6.5(1H, d, J=3.36 Hz), 6.4(1H, q, J=1.86 Hz), | | | | |
| | 4.9(1H, m), 4.2(2H, m), 4.0(8H, m), 3.0(4H, t, J=4.45 Hz), | | | | |
| | 2.6(3H, s). | | | | |
| 67. | 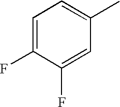 | H | H | Mol. Wt 518 | Yield 68% |
| | 7.6(1H, d, J=15.42 Hz), 7.48(1H, dd, J=2.52 Hz), 11.58 Hz), | | | | |
| | 7.1(2H, m), 7.0(1H, q, J=1.83 Hz), 6.8(2H, d, J=15.36 Hz), 4.3(1H, | | | | |
| | m), 4.0(7H, m), 3.4(1H, s), 3.1(4H, s), 2.6(3H, s) (the solvent used is | | | | |
| | used is CDCl₃ + drops of CD₃OD) | | | | |
| 68. | 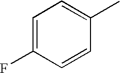 | H | H | Mol. Wt 500 | Yield 49% |
| | 7.7(1H, d, J=15.42 Hz), 7.5(2H, m), 7.1(3H, m), 6.8(1H, d, | | | | |
| | J=15.39 Hz), 6.9(1H, t, J=9.0 Hz), (1H, m), 4.2(1H, m), 4.0(2H, | | | | |
| | m), 3.8(5H, complex), 3.0(4H, t, J=4.4 Hz), 2.6(3H, s) | | | | |
| 69. | 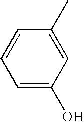 | H | H | Mol. Wt 498 | Yield 9% |
| | 7.47-7.53(1H, dd, J=2.4 Hz), (1H, dd, J=2.4 Hz), 7.38-7.43(1H, d, | | | | |
| | J=15.4 Hz), 7.19-7.21(1H, t, J=4.2, 3.6 Hz), 7.11-7.16(1H, d, | | | | |
| | J=14.7 Hz), 6.76-6.79(1H, dd), 4.92(1H, m), 4.12(1H, t), | | | | |
| | 3.77-3.9(3H, complex), 3.72-3.75(4H, b.t,), 2.98(4H, b.t), | | | | |
| | 2.4(3H, s). (the solvent used is DMSO-d₆) | | | | |
| 70. | 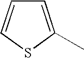 | H | H | Mol. Wt 488 | Yield 70% |
| | 2.60(3H, s), 3.08(4H, m), 3.87(4H, m), 4.02(1H, m), 4.07(H, m), | | | | |
| | 4.11(1H, t, J=4.5 Hz), 4.99(1H, m), 6.68(1H, d, J=15.06 Hz), | | | | |
| | 6.95(1H, t, J=4.56 Hz), 7.06(1H, t, J=6.1 Hz), 7.22(1H, dd, | | | | |
| | J=10.8 Hz), 7.24(1H, d, J=3.4 Hz), 7.32(1H, d, J=5 Hz), 7.42(1H, | | | | |
| | dd, J=16.5 Hz), 7.81(1H, d, J=15 Hz), 7..96(1H, br) | | | | |
| 71. | 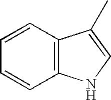 | H | H | Mol. Wt 521 | Yield 31% |
| | 10.35(1H, s), 8.3(1H, d), 7.8(1H, d, J=2.4 Hz), 7.75(1H, d), | | | | |
| | 7.4(1H, d, J=6.9 Hz), 7.1(5H, complex), 4.9(1H, m), 3.8(1H, t), | | | | |
| | 3.7(4H, bt), 3.0(4H, t), 2.4(3H, s). (the solvent used is DMSO-d₆) | | | | |
| 72. | 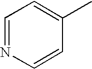 | H | H | Mol. Wt 483 | Yield 47% |
| | 8.7(1H, d, J=1.74 Hz), 8.5(2H, 2, J=1.44 Hz), 7.8 (1H, d), | | | | |
| | 7.7(1H, d, J=15.54 Hz), 7.5(1H, q, J=2.5 Hz, 11.73 Hz), 7.0 (2H, m), | | | | |
| | 5.0(1H, m), 3.8(8H, m), 3.1(4H, t), 2.5(5H, s). | | | | |
| 73. | 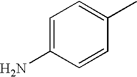 | H | H | Mol. Wt 497 | Yield 8% |
| | 7.61-7.78(1H, d, J=15 Hz), 7.47-7.48(1H, dd, J=2.7 Hz), | | | | |
| | 7.35-7.38(1H, d, J=8.4 Hz), 7.05-7.08(1H, dd, J=1.8, 6.9 Hz), | | | | |
| | 6.89-6.95(1H, t, J=9 Hz), 6.64-6.67(1H, d, J=8.7 Hz), 4.9(1H, m), | | | | |
| | 4.0-4.1(3H, complex), 3.8(4H, br), 3.0(3H, t), 2.5(3H, s). | | | | |
| | (the solvent used is DMSO-d₆) | | | | |
| 74. | 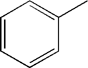 | H | H | Mol. Wt 482 | Yield 79% |
| | 2.56(3H, s), 3.07-3.10(4H, m), 3.78-3.83(4H, m), | | | | |
| | 4.04−4.01(1H, m), 4.10(2H, m), 4.25(1H, t, J=5.9 Hz), 4.97(1H, m), | | | | |
| | 6.88-6.93(1H, t, J=4.48 Hz), 6.95-6.88(1H, d, J=13.23 Hz), | | | | |
| | 7.05(1H, dd, J=10 Hz) 7.37-7.38(5H, m), 7.52(1H, dd, J=15.42 Hz), | | | | |
| | 7.67-7.73(1H, d, J=15.42 Hz), 7.90(1H, bs) | | | | |
| 75. | 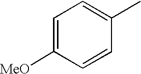 | H | H | Mol. Wt 512 | Yield 80% |
| | (3H, S) 2.68(3H, S) 3.08(4H, m) 3.84(4H, m), 4.01(1H, m), | | | | |
| | 4.06(2H, m), 4.10(1H, t, J=4.35 Hz), 5.0(1H, m), 6.83(1H, d, | | | | |
| | J=15.33 Hz), 6.92(2H, d, J=9 Hz), 6.97(1H, t, J=6.1 Hz), | | | | |
| | 7.09(1H, dd, J=10.62 Hz), 7.42-7.47(1H, dd, J=16.2 Hz), | | | | |
| | 7.51(2H, d, J=8.52 Hz), 7.61(1H, d, J=15.33 Hz), 10(1H, br) | | | | |
| | (the solvent used is CDCl₃ drop of DMSO-d₆) | | | | |
| 76. | 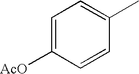 | H | H | Mol. Wt 540 | Yield 63% |
| | 7.76-7.79(2H, d, J-8.4 Hz), 7.52-7.54(1H, t, J=2.4 Hz), | | | | |
| | 7.47-7.49(1H, d, J=4.2 Hz), 7.25-7.30(1H, d, J=15.6 Hz), | | | | |
| | 7.19-7.20(1H, d, J=2.4 Hz), 7.14-7.19(1H, d, J=15 Hz), | | | | |
| | 7.12-7.14(JH, d, J=8.4 Hz), 4.88-4.94(1H, m), 4.09-4.15(1H, t, | | | | |
| | J=9 Hz), 3.7(4H, b), 2.9(4H, br), 2.4(3H, s), 2.2(3H, s). | | | | |
| | (the solvent used is DMSO-d₆) | | | | |
| 77. | 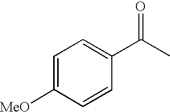 | H | H | Mol. Wt 540 | Yield 15% |
| | 8.06(2H, d, J=2.2 Hz), 7.9-8.0(1H, d, J=15.9 Hz), 7.49-7.53(1H, | | | | |
| | d, J=12 Hz), 7.44-7.49(1H, dd, J=2.7 Hz), 7.06-7.07(1H, d, | | | | |
| | J=1.8 Hz), 4.9(1H, m), 3.87-3.92(4H, t, J=6.6 Hz), 3.07-3.11(4H, | | | | |
| | t, J=5.1 Hz), 2.6(3H, s), 5.0(solvent used is DMSO-d₆) | | | | |
| 78. | 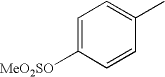 | H | H | Mol. Wt 576 | Yield 40% |
| | 7.6(1H, d, J=15.33 Hz), 7.4(3H, t), 7.1(1H, d, J=8.82 Hz), 6.9(1H, | | | | |
| | t, J=9.0 Hz), 6.8(2H, d, J=8.52 Hz), 6.7(1H, d, J=15.36 Hz), | | | | |
| | 4.9(1H, s), 4.1(3H), 3.1(4H, s), 3.0(3H, s) 2.5(3H, s). | | | | |
| 79 | 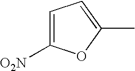 | H | H | Mol. Wt 517 | Yield 36% |
| | 7.4(2H, m), 7.3(2H, q, J=3.78 Hz), 6.9(2H, t, J=9 Hz), 6.7(2H, | | | | |
| | d, J=3.78 Hz), 7.0(1H, dd, J=1.8, 6.9 Hz), 4.9(1H, m), 4.5(2H, s), | | | | |
| | 4.0(4H, m), 3.8(1H, m), 2.6(4H, br), 2.6(3H, s). | | | | |
| 80. | 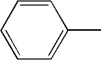 | — | — | Mol. Wt. 480 | Yield 44% |

7.57(2H, dd, J=1.4, 6.51 Hz), 7.42(4H, m), 7.0(1H, dd,
J=8.7 & 1.9 Hz), 6.9(1H, t, J=9 Hz), 4.9(1H, m), 4.3(1H, m),
4.08(2H, t, J=8.85 Hz), 4.01(1H, t, J=5.3 Hz), 3.8(m, 3H), 3.1(2H, t,
J=5.0 Hz), 3.0(2H, t, J=5.0 Hz), 2.6(3H, s)

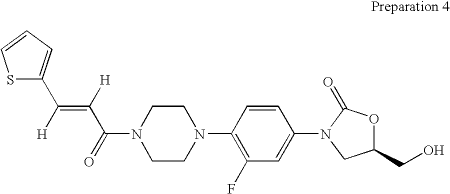

### (S)-N-[3-(3-fluoro-4-{4-[3-(thiophen-2-yl)-acryloyl]-piperazinyl]-phenyl)-2-oxo-oxazolidin-5-yl-methyl alcohol (Compound No. 81)

To a solution of (S)-N-[[3-[3-fluoro-4-(N-piperazinyl)]-phenyl]-2-oxo-5-oxazolidinyl]methyl alcohol (2 g) in dichloromethane (50 ml) was added HOBt.H₂O (1.0 g), 1-(3-Dimethyl aminopropyl)-3-ethyl carbodiimide hydrochloride (1.0 g) followed by 3-(2-thienyl)acrylic acid (1.04 g). The reaction mixture was stirred at ca. 27° C. to which triethylamine (4 ml) was added.

After stirring for 2 hrs. at ca. 27° C. (TLC) the reaction mixture was filtered to give white cake and cake was washed with chilled dichloromethane (20 ml) to afford the title compound (2.13 g, 73%) m.p. 230-235° C.

The following compounds were prepared following the above procedure.

TABLE 4

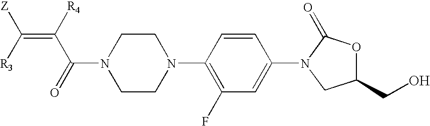

| 81. | Z | R₄ | R₃ | Mol. Wt. | Yield |
|---|---|---|---|---|---|
| | 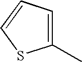 | H | H | 431 | 73% |
| | 7.7(1H), d, J=15.06 Hz), 7.5(1H), dd, J=2.4, 12.6 Hz), 7.4(1H, | | | | |
| | d, J=3.6 Hz), 7.11(1H), dd, J=3.6, 1.5 Hz), 7.0(1H, d, J=9 Hz), | | | | |
| | 6.9(1H, d, J=15 Hz), 4.6(1H, m), 4.0(1H, t, J=9 Hz), | | | | |
| | 3.7(4H, m), 2.9(4H, brs). (the solvent used was DMSO-d₆) | | | | |
| 82. | 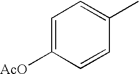 | H | H | Mol. Wt 483 | Yield 86% |
| | 7.76(1H, d, J=8.28 Hz), 7.56(1H, dd, J=17.37 Hz), 7.49(1H, d, | | | | |
| | J=15.43 Hz), 7.21(1H, dd, J=10.12 Hz), 7.17(1H, d, J=15.43 Hz), | | | | |
| | 7.07(1H, t, J=6.1 Hz), 6.76(2H, d, J=8.28 Hz), 5.20(1H, br), | | | | |
| | 4.06(1H, t, J=5.98 Hz), 3.81(4H, m), 3.76(2H, m), 3.63(1H, m), | | | | |
| | 3.03(4H, m), 2.26(3H, s) (the solvent used was DMSO-d₆) | | | | |
| 83. | 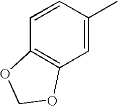 | H | H | Mol. Wt 450 | Yield 85% |
| | 7.56(1H, dd, , J=17.34 Hz), 7.48(1H, m), 7.4(1H, d, J=15.24 | | | | |
| | Hz), 7.19(2H, m), 7.18(1H, dd, J=10.34 Hz), 7.16(1H, t, J=6.2 | | | | |
| | Hz), 7.13(1H, d, J=9.15 Hz), 7.07(1H, d, J=15.24 Hz), 6.94(1H, | | | | |
| | d, J=7.98 Hz), 6.05(2H, s), 4.82(1H, m), 4.03(1H, t, J=6.01 Hz), | | | | |
| | 3.85(2H, m), 3.78(4H, m), 3.68(1H, m), 2.97(4H, m). | | | | |
| | (the solvent used was DMSO-d₆) | | | | |
| 84. | 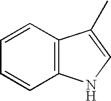 | H | H | Mol. Wt 464 | Yield 95% |
| | 10.71(1H, s)., 7.98(1H, d, J=15.27 Hz), 7.57(1H, dd, J=17.34 | | | | |
| | Hz), 7.47(1H, m), 7.44(2H, m), 7.22(2H, m), 7.10(1H, dd, | | | | |
| | J=10.80 Hz), 7.09(1H, t, J=6.39 Hz), 6.88(1H, d, J=15.27 Hz), | | | | |
| | 4.7(1H, m), 4.00(1H, t, J=6.15 Hz), 3.90(2H, m), 3.84(4H, m), | | | | |
| | 3.70(1H, m), 3.10(4H, m). (solvent used is CDCl₃ + DMSO) | | | | |
| 85. | 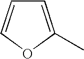 | H | H | Mol. Wt 415 | Yield 79% |
| | 7.78(1H, d, J=1.2 Hz), 7.55(1H, dd, J=17.36 Hz), 7.38(1H, d, | | | | |
| | J=15.19 Hz), 7.18(1H, dd, J=10.74 Hz), 7.09(1H, t, J=6 Hz), | | | | |
| | 6.97(1H, d, J=15.19 Hz), 6.87(1H, d, J=3.31 Hz), 6.60(1H, m), | | | | |
| | 5.21(1H, br), 4.80(1H, m), 1.06(1H, t, J=5.99 Hz), 3.80(4H, m), | | | | |
| | 3.64(2H, m), 3.55(1H, m), 2.97(4H, m) (the solvent used was | | | | |
| | DMSO-d₆) | | | | |
| 86. | 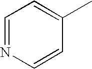 | H | H | Mol. Wt 426 | Yield 84 |
| | 8.61(2H, d, J=5.88 Hz), 7.70(2H, d, J=6.0 Hz), 7.56(1H, d, | | | | |
| | J=15.40 Hz), 7.50(1H, dd, J=14.90 Hz,), 7.43(1H, d, J=15.40 Hz), | | | | |
| | 7.22(1H, dd, J=10.84 Hz), 7.04(1H, t, J=6.20 Hz), 5.20(1H, brs), | | | | |
| | 4.68(1H, m), 4.06(1H, t, J=5.98 Hz), 3.87(2H, m), 3.77(4H, m), | | | | |
| | 3.61(1H, m), 2.99(4H, m) (the solvent used was DMSO-d₆) | | | | |
| 87. | 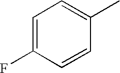 | H | H | Mol. Wt 443 | Yield 74% |
| | δ 7.81(1H, d, J=8.7 Hz), 7.56(2H, d, J=8.7 Hz), 7.50(1H, dd, | | | | |
| | J=17.31 Hz), 7.29(2H, d, J=8.7 Hz) 7.27(1H, dd, J=10.11 Hz), | | | | |
| | 7.21(1H, d, J=14.35 Hz), 7.18(1H, t, J=6.31 Hz), 4.82(1H, m), | | | | |
| | 4.03(1H, t, J=6.12 Hz), 3.80(2H, m), 3.77(4H, m), 3.72(1H, m), | | | | |
| | 2.98(4H, m). (the solvent used was DMSO-d₆) | | | | |
| 88. | 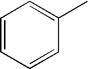 | H | H | Mol. Wt 425 | Yield 83% |
| | 7.71(1H, d, J=14.71 Hz), 7.54(5H, m), 7.50(1H, dd, J=17.05 Hz), | | | | |
| | 7.38(1H, d, J=14.71 Hz), 7.28(1H, dd, J=10.15 Hz), 7.08(1H, t, | | | | |
| | J=6.19 Hz), 4.8(1H, m), 4.03(1H, t, J=6.01 Hz), 3.80(2H, m), | | | | |
| | 3.75(1H, m), 3.62(4H, m), 2.98(4H, m) (the solvent used was | | | | |
| | DMSO-d₆). | | | | |
| 89. | 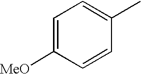 | H | H | Mol. Wt 455 | Yield 86% |

7.69(1H,. d, J=8.74 Hz), 7.55 (1H, dd, J=10.7 Hz), 7.45(1H, d,
J=15.51 Hz), 7.19(1H, d, J=15.51 Hz), 7.18(1H, dd, J=11.77 Hz),
7.12(1H, t, J=5.28 Hz), 6.94(2H, d, J=8.74 Hz), 4.68(1H, m),
4.0(1H, t, J=6.68 Hz), 3.84(2H, m), 3.71(1H, m), 3.62(4H, m),
3.01(4H, m). (the solvent used was DMSO-d₆)

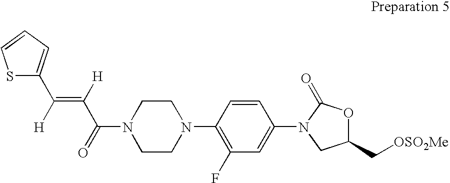

### (S)-N-[3-fluoro-4-{4-[3-(thiophen-2-yl)-acryloyl]-piperazinyl]-phenyl]-2-oxo-oxazolidin-5-yl-methyl methano sulfonate (Compound No. 90)

(S)-N-[3-(3-Fluoro-4-{4-(3-(thiophen-2-yl)-acryloyl}-piperazinyl]-phenyl)-2-oxo-oxazolidin-5-yl-methyl alcohol (2 g) was taken in pyridine (10 ml) and dichloromethane (25 ml) to which was added triethylamine (10 ml). The reaction mixture was cooled to 5° C. and methane sulfonyl chloride (1.5 ml) was added slowly. The reaction mixture was stirred for 3 hrs. at 0-5° C. (TLC). The reaction mixture was washed with DM water (50 ml). The organic layer was separated and dried over anhy. sodium sulfate. After evaporation of solvents the residue was titurated with diethyl ether to afford the title compound as brown solid (2.14 g, 90%) mp. 166-170° C.

The following compounds prepared following the above procedure.

TABLE 5

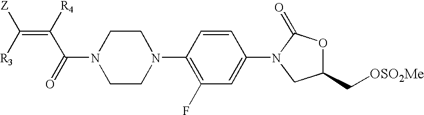

| 90. | Z | R₄ | R₃ | Mol. Wt. | Yield |
|---|---|---|---|---|---|
| | 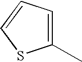 | H | H | 509 | 90% |
| | 7.8(1H, d, J=15.06 Hz), 7.4(1H, dd, J=2.4, 11.7 Hz), 7.3(1H, d, | | | | |
| | J=4.8 Hz), 7.2(1H, d, J=3.3 Hz), 7.0(1H, dd, J=9, 6 Hz), | | | | |
| | 6.7(1H, d, J=15.06 Hz), 4.9(1H, m), 4.1(1H, t, J=9 Hz), | | | | |
| | 3.8(4H, m), 3.15(4H, m), 3.10(3H, s). (the solvent used was | | | | |
| | DMSO-d₆) | | | | |
| 91. | 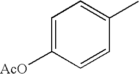 | H | H | Mol. Wt 561 | Yield 75% |
| | 7.71(1H, d, J=15.42 Hz)., 7.56(2H, d, J=8.55 Hz), 7.44(1H, dd, | | | | |
| | J=17.32 Hz), 7.26(1H, dd, J=10.41 Hz), 7.10(2H, d, J=8.55 Hz), | | | | |
| | 6.97(1H, t, J=6.62 Hz), 6.88(1H, d, J=15.42 Hz), 4.92(1H, m), | | | | |
| | 4.47(2H, m), 4.15(1H, t, J=6.1 Hz), 3.82(4H, m), 3.75(1H, | | | | |
| | m), 3.10(4H, m), 3.07(3H, s), 2.32(3H, s) (the solvent used | | | | |
| | was DMSO-d₆) | | | | |
| 92. | 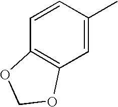 | H | H | Mol. Wt 547 | Yield 83% |
| | 7.58(1H, d, J=18.15 Hz), 7.48(1H, dd, J=14.16 Hz), 7.14(1H, dd, | | | | |
| | J=12.18 Hz), 7.10(1H, dd, J=9.81 Hz), 7.03(1H, t, J=5.91 Hz), | | | | |
| | 6.97(1H, d, 9.11 Hz), 6.83(1H, d, J=18.15 Hz), 6.80(1H, d, J=8.1 | | | | |
| | Hz), 6.01(2H, s), 4.92(1H, m), 4.47(2H, m), 4.20(1H, t, J=6.14 | | | | |
| | Hz), 3.93(1H, m), 3.84(4H, m), 3.14(3H, s), 3.08(4H, m). | | | | |
| | solvent used is DMSO-d₆) | | | | |
| 93. | 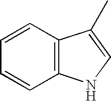 | H | H | Mol. Wt 542 | Yield 85% |
| | 10.72(1H, s), 8.0(1H, d, J=15.30 Hz), 7.50(1H, dd, J=16.74 Hz), | | | | |
| | 7.44(1H, m), 7.42(2H, m), 7.26(2H, m), 7.10(1H, dd, J=10.21 | | | | |
| | Hz), 6.98(1H, t, J=6.12 Hz), 6.92(1H, d, J=15.30 Hz), 4.92 (1H, | | | | |
| | m), 4.43(2H, m), 4.13(1H, t, J=5.06 Hz), 3.90(1H, m), | | | | |
| | 3.49(1H, m), 3.14(3H, m), 3.10(4H, m) | | | | |
| 94. | 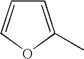 | H | H | Mol. Wt 493 | Yield 83% |
| | 7.52(1H, d, J=15.06 Hz), 7.49(1H, dd, J=17.37 Hz), 7.45(1H, d, | | | | |
| | J=3.33 Hz), 7.09(1H, t, J=10.41 Hz), 6.96(1H, t, J=6.02 Hz), | | | | |
| | 6.85(1H, d, J=15.06 Hz), 6.56(1H, d, 3.33 Hz), 6.45(1H, m), | | | | |
| | 4.92(1H, m), 4.45(1H, m), 4.15(1H, t, J=6.08 Hz), 3.93(1H, m), | | | | |
| | 3.90(4H, m), 3.10(4H, m), 3.08(3H, s). | | | | |
| 95. | 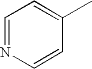 | H | H | Mol. Wt 504 | Yield 100% |
| | 7.59(1H, d, J=15.59 Hz), 8.61(2H, d, J=15.76 Hz), 7.70(2H, d, | | | | |
| | J=6.03 Hz), 7.48(1H, dd, J=15.11 Hz), 7.43(1H, d, J=15.59 Hz), | | | | |
| | 7.09(1H, t, J=6.18 Hz), 5.0(1H, m), 4.45(2H, m), 4.18(1H, t, | | | | |
| | J=6.22 Hz), 3.82(1H, m), 3.73(4H, m), 3.24(3H, s), 3.0(4H, m) | | | | |
| | (the solvent used was DMSO-d₆) | | | | |
| 96. | 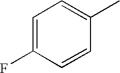 | H | H | Mol. Wt 521 | Yield 94% |
| | 7.67(1H, d, J=15.97 Hz), 7.56(2H, d, J=8.7 Hz), 7.47(1H, | | | | |
| | dd, J=16.65 Hz), 7.13(2H, d, J=8.7 Hz), 7.10(1H, dd, J=10.71 Hz), | | | | |
| | 7.10(1H, t, J=5.69 Hz), 6.85(1H, d, J=15.39 Hz), 4.95(1H, m), | | | | |
| | 4.46(2H, m), 4.18(1H, t, J=6.12), 3.94(1H, m), 3.89(4H, m), | | | | |
| | .87(3H, s), 3.09(4H, m). (the solvent-used was DMSO-d₆) | | | | |
| 97. | 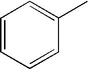 | H | H | Mol. Wt 503 | Yield 95% |
| | 7.73(1H, d, J=15.42 Hz), 7.52(2H, m), 7.45(1H, dd, J=17.3 Hz), | | | | |
| | 7.38(3H, m), 7.26(1H, dd, J=10.8 Hz), 6.97(1H, t, J=6.09 Hz), | | | | |
| | 6.89(1H, d, J=15.42 Hz), 4.91(1H, m), 4.45(2H, m), 4.12(1H, t, | | | | |
| | J=6.1 Hz, 3.95(1H, m), 3.92(4H, m); 3.15(3H, s), 3.08(4H, m). | | | | |
| 98. | 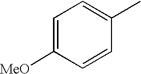 | H | H | Mol. Wt 533 | Yield 91% |

7.70(1H, d, J=15.33 Hz), 7.50(2H, d, J=8.74 Hz), 7.45(1H, dd,
J=17.32 Hz), 7.11(1H, dd, J=10.62 Hz), 6.97(1H, t, J=5.14 Hz),
6.94(2H, d, J=8.74 Hz), 6.76(1H, d, J=15.36 Hz), 4.91(1H, m),
4.45(2H, m), 4.15(1H, t, J=6.08 Hz), 3.95(1H, m), 3.93(3H, s),
3.90(4H, m), 3.14(3H, s), 3.07(4H, m).

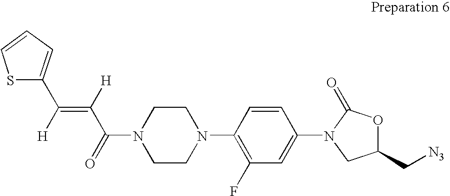

### (S)-N-[3-(3-Fluoro-4-{4-[3-(thiophen-2-yl)-acryloyl]-piperazinyl]-phenyl)-2-oxo-oxazolidin-5-yl-methyl azide. (Compound No. 99)

(S)-N-[3-(3-Fluoro-4-{4-[3-(thiophen-2-yl)-acryloyl]-piperazinyl]-phenyl)-2-oxo-oxazolidin-5-yl-methyl methane sulphonate (2 g) was taken in dimethyl formamide (38 ml) and sodium azide (0.97 g) was added. The reaction mixture was heated to 70-75° C. over a period of 3 hrs. (TLC) and cooled to ca 30° C. The mixture was diluted with ethylacetate (500 ml) and washed with DM water (200 ml). The organic layer was separated and dried over anhydrous sodium sulphate. After evaporation of solvents, the residue obtained was triturated with petroleum ether to afford the title compound as an offwhite solid (1.5 g, 83%), mp 164-172° C.

The following compounds were synthesized following the above procedure.

TABLE 6

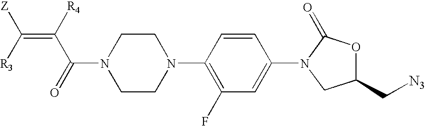

| 99. | Z | R₄ | R₃ | Mol. Wt. | Yield |
|---|---|---|---|---|---|
| | 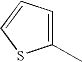 | H | H | 456 | 83% |
| | 7.8(1H, d, J=15.06 Hz), 7.4(1H, dd, J=2.4, 11.7 Hz), 7.3(1H, d, | | | | |
| | J=5.1 Hz), 7.2(1H, d, J=3.3 Hz), 7.0(1H, dd, J=3.6, 4.9 Hz), | | | | |
| | 6.9(1H, t), 6.6(1H, d, J=15 Hz), 4.7(1H, m), 4.0(1H, t, J=9 Hz), | | | | |
| | 3.7(1H, m), 3.1(1H, m) (the solvent used was DMSO-d₆) | | | | |
| 100. | 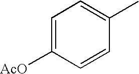 | H | H | Mol. Wt 508 | Yield 80% |
| | 7.71(1H, d, J=15.43 Hz), 7.66(2H), d, J=8.52 Hz), 7.50(1H, dd, | | | | |
| | J=17.31 Hz), 7.10(1H, d, J=8.52 Hz), 6.97(1H, dd, J=10.27 Hz), | | | | |
| | 6.09(1H, t, J=6.1 Hz), 6.83(1H, d, J=15.42 Hz), 4.92(1H, m), | | | | |
| | 4.08(1H, t, J=5.95 Hz), 3.80(4H, m), 3.68(1H, m), 3.09(4H, m), | | | | |
| | 2.33(3H, s) (the solvent used was DMSO-d₆) | | | | |
| 101. | 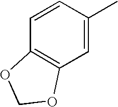 | H | H | Mol. Wt 594 | Yield 89% |
| | 7.65(1H, d, J=15.24 Hz), 7.50(1H, dd, J=16.5 Hz), 7.14(1H, dd, | | | | |
| | J=10.11 Hz), 7.10(1H, dd, J=11.16 Hz), 7.03(1H, t, J=6.12 Hz), | | | | |
| | 6.96(1H, d, J=9.15 Hz), 6.79(1H, d, J=8.11 Hz), 6.76(1H, d, | | | | |
| | J=15.24 Hz), 6.0(2H, s), 4.78(1H, m), 4.08(1H, t, J=5.92 Hz), | | | | |
| | 3.85(2H, m), 3.82(4H, m), 3.73(1H, m), 3.10(4H, m) | | | | |
| 102. | 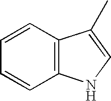 | H | H | Mol. Wt 489 | Yield 86% |
| | [symbol] H, s), 7.99(1H, d, J=15.3 Hz), 7.58(1H, dd, J =17.31 Hz), | | | | |
| | 7.48(1H, m), 7.45(2H, m), 7.23(2H, m), 7.10(1H, dd, J=17.31 | | | | |
| | Hz), 7.09(1H, t, J=6.0 Hz), 6.90(1H, J=15.3 Hz), 4.8(1H, m), | | | | |
| | 4.05(1H, t, J=6.11 Hz), 3.92(2H, m), 3.89(4H, m), 3.72(1H, m), | | | | |
| | 3.1(4H, m). | | | | |
| 103. | 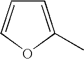 | H | H | Mol. Wt 440 | Yield 84% |
| | 7.52(1H, d, J=15.43 Hz), 7.49(1H, dd, J=17.34 Hz), 7.45(1H, d, | | | | |
| | J=3.2 Hz), 7.10(1H, dd, J=10.37 Hz), 6.93(1H, t, J=6.03 Hz), | | | | |
| | 6.80(1H, d, J=15.43 Hz), 6.57(1H, d, J=3.3 Hz), 6.45(1H, m), | | | | |
| | 4.92(1H, m), 4.08(1H, t, J=5.94 Hz), 3.90(2H, m), 3.90(4H, m), | | | | |
| | 3.73(1H, m), 3.09(4H, m) | | | | |
| 104. | 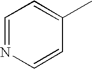 | H | H | Mol. Wt 451 | Yield 84% |
| | 8.61(2H, d, J=5.88 Hz), 7.70(2H, d, J=5.99 Hz), 7.59(1H, d, | | | | |
| | J=15.66 Hz), 7.54(1H, dd, J=1,4.8 Hz), 7.43(1H, d, J=15.6 Hz), | | | | |
| | 7.22(1H, dd, J=11.0 Hz), 7.09(1H, t, J=6.17 Hz), 4.89(1H, m), | | | | |
| | 4.07(1H, t, J=6.10 Hz), 3.77(2H, m), 3.72(4H, m), 3.65(1H, m), | | | | |
| | 3.0(4H, m) (the solvent used was DMSO-d₆) | | | | |
| 105. | 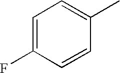 | H | H | Mol. Wt 468 | Yield 70% |
| | 7.69(1H, d, J=15.42 Hz), 7.54(2H, d, J=8.7 Hz), 7.44(1H, dd, | | | | |
| | J=16.68 Hz), 7.11(2H, d, J=8.7 Hz), 7.07(1H, dd, J=10.11 Hz), | | | | |
| | 6.93(1H, t, J=6.11 Hz), 6.80(1H, d, J=15.42 Hz), 4.78(1H, m), | | | | |
| | 4.08(1H, t, J=5.93 Hz), 3.90(2H, m), 3.80(4H, m), 3.60(1H, m), | | | | |
| | 3.09(4H, m). | | | | |
| 106. | 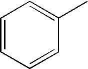 | H | H | Mol. Wt 462 | Yield 85% |
| | 7.73(2H, m), 7.59(1H, dd, J=15.14 Hz), 7.38(3H, m), 7.22(1H, | | | | |
| | d, J=15.14 Hz) 7.18(1H, dd, J=9.78 Hz), 7.09(1H, t, J=6.01 Hz), | | | | |
| | 4.88(1H, m), 4.10(1H, t, J=6.10 Hz), 3.86(4H, m), 3.73(2H, m), | | | | |
| | 3.65(1H, m), 2.99(4H, m) (the solvent used was DMSO-d₆) | | | | |
| 107. | 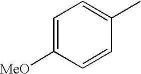 | H | H | Mol. Wt 492 | Yield 84% |

7.65(1H, d, J=15.3 Hz), 7.50(2H, d, J=8.64 Hz), .44(1H, dd,
J=16.2 Hz), 7.04(1H, dd, J=9.14 Hz), 6.94(2H, d, J=8.64 Hz),
6.91(1H, t, J=6.02 Hz) 6.80(1H, d, J=15.2 Hz), 4.9(1H, m),
4.32(1H, t, J=6.1), 3.98(4H, m), 3.92(2H, m), 3.89(1H. m),
3.07(4H, m) (the solvent used was DMSO-d₆)

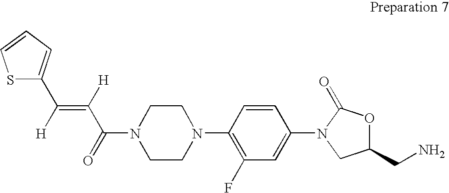

### (S)-N-[3-(3-Fluoro-4-{4-[3-(thiophen-2-yl)-acryloyl]-piperazinyl}-phenyl)-2-oxo-oxazolidin-5-yl-methyl amine (Compound No. 108)

(S)-N-[3-(3-Fluoro-4-{4-[3-(thiophen-2-yl)-acryloyl]-piperazinyl]-phenyl)-2-oxo-oxazolidin-5-yl-methyl azide (1.25 g) and triphenylphosphine (0.860 g) were taken in a mixture of 1,4-dioxane:methanol (25 mL:5 mL) at ca 27° C. and stirred for 1 hour. To this was added aqueous ammonia (8 mL) at ca 27° C. and stirred for another 1 hour (TLC). The solvents were removed under reduced pressure to afford crude oil, which was triturated with diisopropyl ether to afford title compound (1 g, 85%), m.p. 195-200° C.

The following compounds were made following above procedure.

TABLE 7

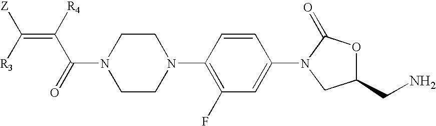

| 108. | Z | R₄ | R₃ | Mol. Wt. | Yield |
|---|---|---|---|---|---|
| | 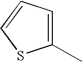 | H | H | 430 | 85% |
| | 7.8(1H, d, J=15.06 Hz), 7.5(1H, dd, J=2.7, 11.7 Hz), 7.3(1H, d, | | | | |
| | J=5.1 Hz), 7.2(1H, d, J=3.3 Hz), 7.1(1H, dd, J=2.4 Hz), | | | | |
| | 7.03(1H, t, J=4.8, 3.6 Hz), 6.6(1H, d, J=15.06 Hz), 4.6(1H, m), | | | | |
| | 4.0(1H, t, J=8.7 Hz), 3.8(4H, m), 3.0(4H, m) (the solvent used | | | | |
| | is DMSO-d₆) | | | | |
| 109. | 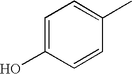 | H | H | Mol. Wt 440 | Yield 69% |
| | 9.67(1H, br), 7.67(1H, d, J=15.42 Hz), 7.53(2H,d, J=7.88 Hz), | | | | |
| | 7.45(1H, dd, J =17.32 Hz), 7.17(1H, dd, J=10.32 Hz), 7.11(1H, | | | | |
| | d, J=15.42 Hz), 7.08(1H, t, J=4.99 Hz), 6.78(2H, d, J=7.88 Hz), | | | | |
| | 4.73(1H, m), 4.11(1H, t, J=5.38 Hz), 3.97(2H, m), 3.82(1H, m), | | | | |
| | 3.80(4H, m), 3.10(4H, m). (solvent used is DMSO-d₆) | | | | |
| 110. | 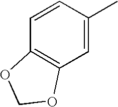 | H | H | Mol. Wt 468 | Yield 80% |
| | 7.65(1H, d, J=15.27 Hz), 7.46(1H, dd, J=16.8 Hz), 7.12(1H, d, | | | | |
| | J=10.12 Hz), 7.0(1H, dd, J=15.27 Hz), 6.96(1H, t, J=6.21 Hz), | | | | |
| | 6.92(1H, d, J=9.15 Hz), 6.79(1H, d, J=7.95 Hz.), 6.71(1H, d, | | | | |
| | J=15.27 Hz), 6.0(2H, s), 4.91(1H, m), 4.04(1H, t, J=5.8 Hz), | | | | |
| | 3.89(2H, m), 3.85(1H, m), 3.80(4H, m), 3.09(4H, m). | | | | |
| | (the solvent used is DMSO-d₆) | | | | |
| 111. | 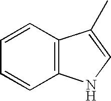 | H | H | Mol. Wt 463 | Yield 80% |
| | 11.65(1H, s), 7.96(1H, d, J=15.3 Hz), 7.76(1H, dd, J=17.32 Hz), | | | | |
| | 7.45(1H, m), 7.42(2H, m), 7.21(2H, m), 7.13(1H, dd, J=10.62 | | | | |
| | Hz), 7.09(1H, t, J=5.88 Hz), 6.96(1H, d, J=15.3 Hz), | | | | |
| | 4.82(1H, m), 4.05(1H, t, J =6.02 Hz), 3.84(4H, m), | | | | |
| | 3.82(4H, m), 3.79(1H, m), 3.0(4H, m). (the solvent used is | | | | |
| | DMSO-d₆) | | | | |
| 112. | 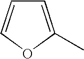 | H | H | Mol. Wt 386 | Yield 82% |
| | 7.54(1H, dd, J=17.37 Hz), 7.49(1H, d, J=2.38 Hz), 7.38(1H, d, | | | | |
| | J=15.17 Hz), 7.18(1H, dd, J=10.18 Hz), 7.06(1H, t, J=6.12 Hz), | | | | |
| | 6.92(1H, d, J=15.17 Hz), 6.86(1H, d, J=2.38 Hz), 6.60(1H, m), | | | | |
| | 4.58(1H, m), 4.04(1H, t, J=4.58 Hz), 3.803(4H, m), | | | | |
| | 3.78(2H, m), 3.146(1H, m), 2.97(4H, m), 3.10(4H, m). | | | | |
| | (the solvent used is DMSO-d₆). | | | | |
| 113. | 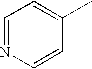 | H | H | Mol. Wt 425 | Yield 82% |
| | 8.61(2H, d, J=8.88 Hz), 7.70(2H, d, J=6 Hz), 7.5(1H, d, J=15.36 | | | | |
| | Hz), 7.54(1H, dd, J=16.38 Hz), 7.48(1H, dd, J=16.38 Hz), | | | | |
| | 7.48(1H, d, J=15.38 Hz), 7.19(1H, dd, J=10.11 Hz), | | | | |
| | 4.59(1H, m), 4.05(1H, m), 4.05(1H, t, J=5.93 Hz), 3.86(2H, m), | | | | |
| | 3.81(4H, m), 3.72(1H, m), 2.98(4H, m). (the solvent used is | | | | |
| | DMSO-d₆) | | | | |
| 114. | 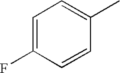 | H | H | Mol. Wt 442 | Yield 82% |
| | 7.70(1H, d, J=15.39 Hz), 7.54(2H, d, J=8.43 Hz), 7.47(1H, dd, | | | | |
| | J=16.11 Hz), 7.12(2H, d, J=8.43 Hz), 7.07(1H, dd, J=9.78 Hz), | | | | |
| | 6.93(1H, t, J=6.13 Hz), 6.81(1H, d, J=15.39 Hz), 4.66(1H, m), | | | | |
| | 4.04(1H, t, J=5.8 Hz), 3.98(2H, m), 3.83(4H, m), 3.80(1H, m), | | | | |
| | 3.10(4H, m) | | | | |
| 115. | 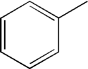 | H | H | Mol. Wt 436 | Yield 76% |
| | 7.73(2H, m), 7.54(1H, d, J=15.31 Hz), 7.42(1H, dd, | | | | |
| | J=16.37 Hz), 7.38(3H, m), 7.29(1H, d, J=15.31 Hz), 7.18(1H, | | | | |
| | dd, J=10.86 Hz), 7.05(1H, t, J=6.15 Hz), 4.64(1H, m), | | | | |
| | 4.08(1H, t, J=5.97 Hz), 3.85(4H, m), 3.81(2H, m), 3.72(1H, m), | | | | |
| | 2.98(4H, m) (the solvent used is DMSO-d₆) | | | | |
| 116. | 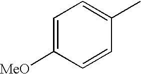 | H | H | Mol. Wt 464 | Yield 75% |

7.676(1H, d, J=15.4 Hz), 7.52(2H, d, J=8.52 Hz), 7.41(1H, dd,
J=17.2 Hz), 7.08(1H, dd, J=10.14 Hz), 7.1(2H, d, J=8.52 Hz),
6.98(1H, t, J=6.1 Hz), 6.78(1H, d, J=15.4 Hz), 4.9(1H, m),
4.25(1H, t, J=6.2 Hz), 4.11(4H, m), 3.99(2H, m), 3.90(1H, m)
3.09(4H, m)

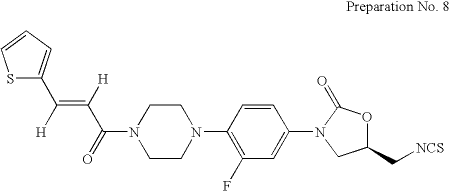

### (S)-N-[3-(3-Fluoro-4-{4-[3-(thophen-2-yl)-acryloyl]-piperazinyl]-phenyl)-2-oxo-oxazolidin-5-yl-methyl thioisocynate (Compound No. 117)

A mixture of (S)-N-[3-(3-Fluoro-4-{4-[3-thiophen-2yl)-acryloyl]-piperazinyl]-phenyl)-2-oxo-oxazolidin-5-yl methyl amine (1 g) CS₂ (0.13 ml) and Et₃N (0.5 ml) in THF (10 ml) was stirred at ca 30° C. for 5 hours. Then ethyl chloroformate (0.30 ml) was added to the mixture and stirred at the same temp for 1 hour (TLC). The mixture was qunched with DM water (25 ml) and extracted with EtOAc (100 ml). The extract was washed with brine (25 ml), again separated the organic layer, dried and concentrated under vacuum initially afforded an oil, which was triturated with diisopropyl ether to give title compound (1 g, 91%).

The following compounds were made following above procedure

TABLE 8

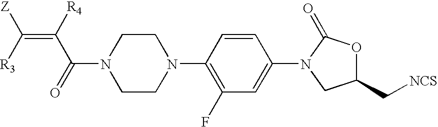

| 117. | Z | R₄ | R₃ | Mol. Wt. | Yield |
|---|---|---|---|---|---|
| | 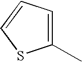 | H | H | 472 | 91% |
| | 7.8(1H, d, J=15.06 Hz), 7.4(1H, dd, J=2.4 Hz), 7.3(1H, d, J=5.1 Hz), 7.2(1H, t, J=8.1, 3.3 Hz), 7.0(1H, dd, J=3.9, 1.2 Hz), 6.9(1H, t, J=9.3 Hz), 6.6(1H, d, J=15.06 Hz), 4.8 (1H, m), 4.1(1H, t, J=9 Hz), 3.8(4H, m), 3.09(4H,m). (solvent used is DMSO-d₆) | | | | |
| 118. | 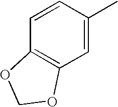 | H | H | Mol. Wt 450 | Yield 86% |
| | 7.4(1H, dd, J=6.12 Hz), 7.0(2H, d, J=8.7 Hz), 6.9(1H,m), 6.0 (1H, t), 4.7(1H, m), 4.0(1H, t), 2.02(3H, s), 3.0(8H, complex) 3.7(3H, m) | | | | |
| 119. | 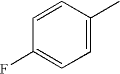 | H | H | Mol. Wt 484 | Yield 32% |

7.8(2H, d, J=8.59 Hz), 7.53(1H, d, J=15 Hz), 7.48(1H, dd, J=16.31 Hz) 7.23(2H,d), 7.13(1H, dd, J=10 Hz), 7.06(1H, t) 4.96(1H, m), 4.08(1H, t), 3.85(4H, m), 3.75(1H, m), 3.44(2H, m), 3.09(4H, m) (solvent used is DMSO-d₆)

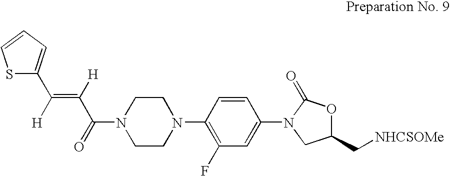

### (S)-N-[3-(3-Fluoro-4-{4-{3-(thiophen-2-yl)-acryloyl]-piperzinyl}-phenyl)-2-oxo-oxazoldin-5-yl-methyl thiocarbamate (Compound No. 120).

To solution of NaH (60% in oil, 0.10 g) in methanol (10 ml), a mixture of compound No. 117 (1 g) in methanol (10 ml) was added under ice cooling followed by stirring of ca 27° C. for 3 h (TLC). The reaction mixture was poured into ice water and adjusted pH 7 with dilute HCl. The solid collected was purified through column chromatography using eluant 0-4% methanol in CHCl₃. The solution was concentrated to afford the title compound (300 mg, 29%) mp 180-185° C.

The following compounds were made following above procedure.

TABLE 9

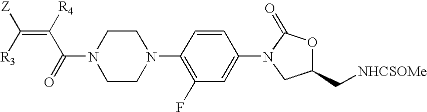

| 120. | Z | R₄ | R₃ | Mol. Wt. | Yield |
|---|---|---|---|---|---|
| | 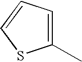 | H | H | 504 | 78% |
| | 7.8(1H, d, J=15.06 Hz), 7.4(1H, d, J=11.7 & 2.7 Hz), 7.3(1H, d, J=5.1 Hz), 7.2 (1H, t, J =6.6 & 3.3 Hz), 6.6(1H, d, J=15.06 Hz), 4.9(1H, m), 4.0(4H, m), 3.8(4H, m) (the solvent used was DMSO-d₆) | | | | |
| 121. | 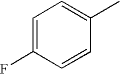 | H | H | Mol. Wt 493 | Yield 83% |
| | 8.7(1H,d, J=1.71 Hz), 8.5(1H, d, J=3.86 Hz), 8.1(1H, d, J=8.04 Hz), 7.6 (2H, d, J=15.57 Hz), 7.2(1H, d, 5=15, 57 Hz), 7.0(1H, t, J=9.12 Hz), 4.8(1H, t, J=9 Hz), 3.5(2H, d, J=4.95 Hz), 1.95 (3H, s), 7.5(2H, m), 7.1(1H, dd, J=1.86 Hz), 4.0(1H, t, J=9.0 Hz), 3.9(4H, t), 3.7(1H, m), 3.1(4H, t), 4.7(1H, m) | | | | |
| 122. | 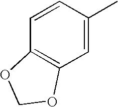 | H | H | Mol. Wt 450 | Yield 86% |

7.4(1H1, dd, J=6.12 Hz), 7.0(2H, d, J=8.7 Hz), 6.9(1H, m), 6.0 (1H, t), 4.7(1H, m,) 4.0(1H, t), 2.02(3H, s), 3.0(SH, complex) 3.7 (3H, m) (the solvent used is DMSO-d₆)

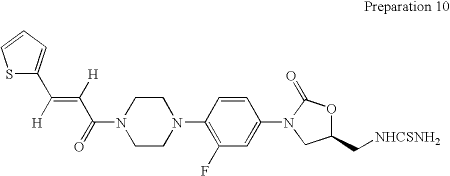

### (3/-N-[3-(3-Fluoro-4-{4-(thiophen-2-yl)-acryloyl]-piperazineyl]-phenyl)-2-oxo-oxazolidin-5-yl-methyl]thiourea (Compound No. 123)

A mixture of (S)-N-[3-(3-Fluoro-4-{4-[3-(thiophen-2-yl)-acryloyl]-piperazinyl]-phenyl)-2-oxo-oxazolidin-5-yl-methylamine (0.5 g) CS₂ (0.09 ml) and Et₃N (0.25 ml) in THF. (5 ml) was stirred at ca 30° C. for 5 hours. Then ethylchloroformate (0.15 ml) was added to the mixture and stirred at ca 30° C. for 1 hour (TLC). The mixture was quenched with DM water (10 ml) and extracted with EtOAc (50 ml). The extract was dried over anhy. Na₂SO₄ and concentrated under vacuum to afford oil (0.5 g) which was taken in methanol (10 ml) and to this stirred solution added a solution of 16% ammonia gas in methanol (10 ml) for 1 hour at Ca 27° C. (TLC) solid began to separate, which was filtered to afford the title compound as white solid (0.25 g, 60%) mp 154-157° C.

The following compounds prepared following the above procedure.

TABLE 10

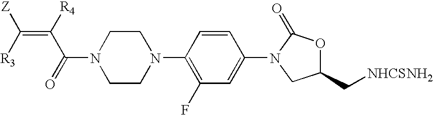

| 123. | Z | R₄ | R₃ | Mol. Wt | Yield |
|---|---|---|---|---|---|
| | H | H | 489 | 60% | |
| | 7.83 (1H, d, J=15.06 Hz), 7.72(1H, br), 7.39(1H, d, J=5.04 Hz), 7.34(1H, dd, J=7.32 Hz), 7.22(1H, d, J=3.4 Hz), 7.07(1H, m), 7.04(1H, dd, J=8.7 Hz), 6.92(1H, t, J=6.1 Hz), 6.72 (1H, d, J=15.06 Hz), 6.32 (2H1, br), 4.91(1H, m), 4.36(1H, t, J=5.98 Hz), 4.11(2H, m), 4.04(1H, m), 3.91(4H, m), 3.06(4H, m) (the solvent used is DMSO-d₆) | | | | |
| 124. | 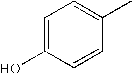 | H | H | Mol. Wt 499 | Yield 40% |
| | 7.90(1H, d, J=15.2 Hz), 7.56(2H, d, J=8.44 Hz), 7.45(1H, dd, 7.03(1HI, d, J=15.2 Hz), 6.78(2H, d, J=8.44 Hz), 4.53(1H, m), 4.08(1H, t, J=5.78 Hz), 4.0(2H, m), 3.98 (1H, m), 3.80(4H, m), 3.31(4H, m) (the solvent used is DMSO-d₆) | | | | |
| 125. | 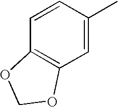 | H | H | Mol. Wt 527 | Yield 45% |
| | 7.61(1H, d, J=15.31 Hz), 7.38(1H, dd, J=15.9 Hz), 7.07(1H, d, J=9.16 Hz), 7.02(1H, dd, J=8.16 Hz), 6.99(1H, s), 6.91(1H, dd, J=10.2 Hz), 6.85(1H, t, J=6.0 Hz), 6.76(1H, d, J=15.3 Hz), 6.71 (2H, br), 5.99(2H, s), 4.90(1H, m), 4.15(1H,t, J=6.11 Hz), 3.89 (1H, m), 3.84(4H, m), 3.05(4H, m). | | | | |
| 126. | 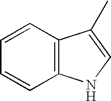 | H | H | Mol. Wt 522 | Yield 53% |
| | 10.38(1H, s), 7.94(1H, d, J=15.27 Hz), 7.51(1H, dd, J=17.32 Hz), 7.47(2H, m), 7.44 (1H,s), 7.35(2H, m), 7.22(1H, dd, J=10.5 Hz), 7.07(1H, t, J=6.05 Hz), 6.94(1H, d, J=15.51 Hz), 6.89(2H, br), 4.87(1H, m), 4.15(1H, t, J=4.76 Hz), 4.08(2H, m), 4.01(1H, m), 3.92(4H, M), 3.10(4H, m) (solvent used is CDCl₃ + DMSO-d6). | | | | |
| 127. | 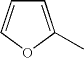 | H | H | Mol. Wt 473 | Yield 40% |
| | 7.72(1H, br), 7.48(1H, d, J=15.02 Hz). 7.45(1H, m), 7.39(1H, dd, J=16.38 Hz), 7.04(1H, dd, J=10.81 Hz), 6.89(1H, t, J=6.1 Hz), 6.79(1H, d, J=15.01 Hz), 6.58(1H1, d, J=3.3 Hz), 6.47(1H, d, J=3.24 Hz), 6.32(2H, br), 4.91(1H, m), 4.08(1H, t, J=5.9 Hz), 4.02(1H, m), 3.91(2H, m), 3.79(4H, m), 3.05(4H, m). | | | | |
| 128. | 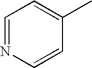 | H | H | Mol. Wt 484 | Yield 53% |
| | 8.61(2H, d, J=5.93 Hz), 7.70(2H, d, J=6.01 Hz), 7.59(1H, d, J=15.35 Hz), 7.54(1H, dd, J=16.28 Hz). 7.48(1H, d, J=15.35 Hz), 7.20(1H, dd, J=10.95 Hz), 7.05(1H, t, J=6.16 Hz), 4.81(1H, m), 4.11(1H, t, J=5.97 Hz), 3.87(2H, m), 3.79(4H, m), 3.50(1H, m), 2.99(4H, m). (solvent used is CDCl₃ + DMS0-d₆). | | | | |
| 129. | 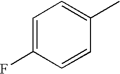 | H | H | Mol. Wt 501 | Yield 79% |
| | 7.80(2H, d, J=9.0 Hz), 7.53(1H, dd, J=17.31 Hz), 7.23(1H, dd, J=10.81 Hz), 7.22(1H. t, J=6.09 Hz), 7.11(1H, d, J=9 Hz), 7.08 (1H, d, J=15.22 Hz), 4.81(1H, m), 4.0(1H, t, J=5.98 Hz), 3.84(4H, m), 3.79(1H, m), 3.73(4H, m), 2.98(4H, m). (solvent used is DMSO-d₆) | | | | |
| 130. | 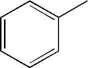 | H | H | Mol. Wt 483 | Yield 67% |
| | 7.68-7.70(1H, d, J=15.42 Hz), 7.52-7.55(1H, dd, J=17.4 Hz), 7.3 (5H, m), 7.0(1H, dd, J=10.56 Hz), 6.9(1H, d, J=15.33 Hz), 6.89(1H, t, J=7.66 Hz), 6.27(2H, br), 4.9(1H, m), 4.60(1H, t, J=5.9 Hz), 4.15(2H, m), 4.10 (2H, m), 4.06(4H, m), 2.99(4H, m). | | | | |
| 131. | 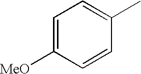 | H | H | Mol. Wt 513 | Yield 70% |

7.6(1H, d, J=15.27 Hz),7.5(1H, d, J=8.52 Hz), 7.3(1H, dd, J=16.08 Hz), 7.0(1H, dd, J=7.14 Hz), 6.9(2H, d, J=8.52 Hz), 6.91(1H, t, J=5.21 Hz), 6.7(1H, d, J=15.3 Hz), 6.2(2H, br), 4.9(1H, m), 4.35(1H, t, J=5.91 Hz), 4.11(2H, m), 3.98(1H, m), 3.9(4H, m), 3.92(3H, s), 3.08(4H, m)

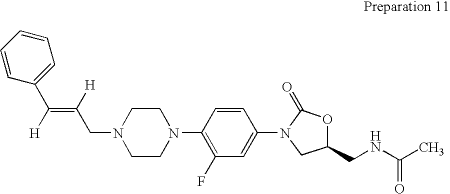

### N-(3-{3-fluoro-4-[4-(3-phenyl-allyl)-piperazin-1-yl]phenyl}-2-oxo-oxazolidin-5-yl-methyl)acetamide. (Compound No. 132)

A mixture of 3-(3-Fluoro-4-piperazinyl-phenyl)-2-oxo-5-oxazolidinyl)acetamide (0.5 g), 10 mL acetone and potassium carbonate (0.205 g) was stirred at ca 27° C. for 1 hour. The Cinnamoyl chloride (0.226 g) was added to this mixture at ca 27° C. and left the reaction mixture overnight (TLC). The mixture was quenched with DM water (25 mL) and extracted with 50 mL of chloroform. The organic layer was separated and dried over anhydrous Na₂SO₄ and concentrated under vacuum to afford an oil. The crude product was purified through column chromatography by using eluent as 0-3.5% MeOH in CHCl₃. The distillation of solvents afforded the title compound as white solid (0.15 g, 22%), m.p 134-136° C.

| | | | | | |
|---|---|---|---|---|---|
| 132. | 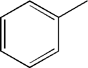 | H | H | Mol. Wt 452 | Yield 44% |

7.45(5H, m), 7.3(2H, t, J=6.99) 7.0(1H, dd, J=2.2, 8.7), 6.9(1H, t, J=9), 6.5(1H, d, J=15.84), 6.0(1H, t), 4.7(1H, m), 4.0(1H, t, J=9), 3.7(3H, m), 3.2(2H, d), 3.1(4H, s), 2.2(4H, s), 2.0(3H, s).

The compounds of the present invention have useful activity against a variety of organisms. The in vitro activity of compounds of the present invention can be assessed by standard testing procedures such as the determination of minimum inhibitory concentration (MIC) by standard “Microdilution method” as described elsewhere in the specification. The pharmacokinetic profiling of the compounds were also done according to the protocol described in this specification. The activities of representative compounds of the present invention are given below in the following table.

Guide to Table Abbreviations:

MRSA: Methicillin resistant Staphylococus aureus 653SP

SE: Staphylococcus epidermidis ATCC 12228

EF: Enterococcus faecalis ATCC 29212

SA: Staphylococus aureus ATCC 33591

TABLE
MIC (μg/ml) in vitro activity in gram positive organisms.

| Sl. No. | Compound No. | SA | SE | EF | SA |
|---|---|---|---|---|---|
| 1. | 01 | 0.5 | 1 | 1 | — |
| 2. | 05 | 1 | 0.5 | 0.5 | 2 |
| 3. | 64 | 0.5 | 0.25 | 0.25 | 0.25 |
| 4. | 66 | 1 | 0.5 | 0.5 | 0.5 |
| 5. | 70 | 1 | 2 | 0.5 | 0.5 |
| 6. | 123 | 1 | 0.5 | 0.25 | 1 |
| 7. | 124 | 1 | 0.5 | 1 | 1 |
| 8. | 125 | 1 | 2 | 0.5 | 4 |
| 9. | 126 | 1 | 0.5 | 1 | 1 |
| 10. | 127 | 2 | 0.5 | 1 | 2 |
| 11 | Linezolid | 2 | 4 | 4 | 4 |
| 12. | Eperzolid | 2 | 4 | 2 | 4 |

We claim:

1. A compound of formula (I), their analogs, their stereoisomers, tautomeric forms, their pharmaceutically acceptable salts, their pharmaceutically acceptable solvates, and pharmaceutical compositions containing them, 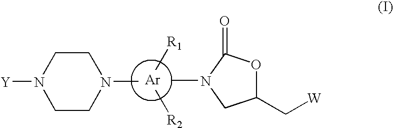
Where Ar represents an optionally substituted phenyl ring, five or six membered hetero aromatic ring which may be substituted or unsubstituted; R₁ & R₂ may be same or different and represent hydrogen, halogen, substituted or unsubstituted groups selected from alkyl, aralkyl, alkoxy, thio, amino, aminoalkyl, nitro, cyano, formyl, thioalkoxy, cycloalkyl, haloalkyl, haloalkoxy, groups;
Y represents the groups G₁, G₂ or G₃: 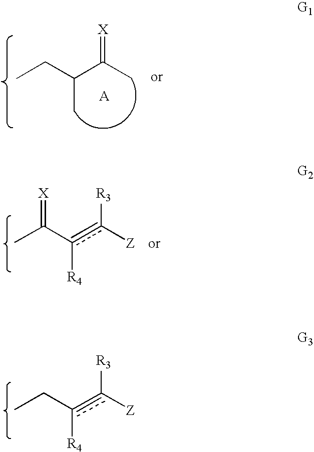
where R₃ & R₄ may be same or different and represent H, C₁-C₆ substituted or unsubstituted linear or branched alkyl group, halogen, hydroxy, cyano, haloalkyl, haloalkoxy, perhaloalkoxy, thio, substituted or unsubstituted groups selected from cycloalkyl, (C₁-C₁₂)alkoxy, cyclo(C₃-C₇)alkoxy, aryl, aryloxy, aralkyl, ar(C₁-C₁₂)alkoxy, acyl, acyloxy, carboxylic acid and its derivatives such as esters and amides, hydroxyalkyl, aminoalkyl, mono-substituted or di-substituted aminoalkyl, alkoxyalkyl, aryloxyalkyl, aralkoxyalkyl, (C₁-C₁₂)alkylthio, thio(C₁-C₁₂)alkyl & arylthio; X represents O, S or NR⁵ where R⁵ represents H or (un)substituted alkyl or aryl groups; A represents a (un)substituted, saturated or unsaturated or partially saturated single or fused ring moiety, optionally containing one or more heteroatoms selected from N, S, O; Z represents H, C₁-C₆ substituted or unsubstituted alkyl group, cyano, haloalkyl, haloalkoxy, perhaloalkoxy, substituted or unsubstituted groups selected from cycloalkyl, bicycloalkyl, (C₁-C₁₂)alkoxy, cyclo(C₃-C₇)alkoxy, aryl, aryloxy, aralkyl, ar(C₁-C₁₂)alkoxy, heterocyclyl, heteroaryl, heterocyclyl(C₁-C₁₂)alkyl, heteroar(C₁-C₁₂)alkyl, heteroaryloxy, heteroar(C₁-C₁₂)alkoxy, heterocycloxy, heterocyclylalkyloxy, acyl, acyloxy, acylamino, carboxylic acid and its derivatives such as esters and amides, hydroxyalkyl, aminoalkyl, mono-substituted or di-substituted aminoalkyl, alkoxyalkyl, aryloxyalkyl, aralkoxyalkyl, (C₁-C₁₂)alkylthio, thio(C₁-C₁₂)alkyl, arylthio, SOR₆ and SO₂R₆, where R₆ represents amino, optionally substituted groups selected from alkyl, aryl, heteroaryl, heterocyclyl groups; the dotted line ‘------’ represents either a bond or a no bond.
W represents OH, N₃, NH₂, NCS, OSO₂CH₃ or a moiety of general formula 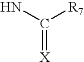
Wherein R₇ may be H, substituted or unsubstituted groups selected from amino, alkylamino, dialkylamino, aralkylamino, C₁-C₆alkoxy, C₁-C₁₂alkyl, aralkyl, C₃-C₁₂cycloalkyl, C₁-C₆thioalkyl, C₁-C₆haloalkyl, thioalkoxy, and X is selected from O, S, —NR₅ where R₅ represents H, or substituted or unsubstituted alkyl group or aryl groups.

2. A compound as defined in claim 1 wherein substituents on groups A & Z are selected from cyano, nitro, halo, perhaloalkyl, carboxyl, hydrazino, azido, formyl, amino, thio, hydroxy, sulfenyl, or substituted or unsubstituted groups selected from alkyl which may be linear or branched; cycloalkyl, alkenyl, cycloalkenyl, alkynyl, hydrazinoalkyl, alkylhydrazido, hydroxylamino, acyl, acyloxy, acylamino, carboxyalkyl, haloalkyl, aminoalkyl, haloalkoxy, hydroxyalkyl, alkoxyalkyl, thioalkyl, alkylthio, alkylsulfinyl, alkylsulfonyl, alkylaminoalkyl, arylamino, alkylamino, aralkylamino, aralkoxy, haloaralkyl, aralkenyl, aryl, aralkyl, aryloxy, alkoxy, alkylcarbonyl, alkoxycarbonyl, aryloxycarbonyl, aralkoxycarbonyl, alkylcarbonylalkyl, alkoxycarbonylalkyl, 1-alkoxycarbonyloxy-alkyl, 1-cycloalkyloxycarbonyloxy-alkyl, carboxamidoalkyl, cyanoamidino, cyanoalkyl, aminocarbonylalkyl, N-aminocarbonylalkyl, N-arylaminocarbonyl, N-alkyl-N-arylaminocarbonyl, carboxyalkylaminocarboxy, N-alkylamino, N,N-dialkylamino, N-arylamino, N-aralkylamino, N-alkyl-N-aralkylamino, N-alkyl-N-arylamino, N-alkylaminoalkyl, N,N-dialkylaminoalkyl, N-arylaminoalkyl, N-aralkylaminoalkyl, N-alkyl-N-aralkylaminoalkyl, N-aralkyl-N-alkylaminoalkyl, N-alkyl-N-arylaminoalkyl, N,N-dialkylaminocarbonyl, N-alkyl-N-arylaminocarbonyl, N-alkyl-N-hydroxyaminocarbonyl, N-alkyl-N-hydroxyaminocarbonylalkyl, alkoxyalkyl, aryloxyalkyl, aralkoxyalkyl, arylthio, aralkylthio, alkoxycarbonyl, aminocarbonyl, alkoxycarbonylamino, cycloalkyl, bicycloalkyl, cycloalkoxy, bicycloalkenyl, heterocyclyl, heterocyclylalkyl, heteroaryl, heteroaralkyl, heteroaryloxy, heteroaralkoxy, heterocyclylalkyloxy, heterocycloalkoxycarbonyl, heteroaryloxycarbonyl, heteroaralkoxycarbonyl, RSO₂NH— and RSO₂O— groups wherein R represents alkyl, aryl, heteroaryl, aralkyl, heteroaralkyl, heterocyclyl, heterocyclylalkyl groups.

3. A compound as claimed in claim 1 where R¹ is hydrogen and R² is halo.

4. A compound as claimed in claim 1 where Ar represents a phenyl ring.

5. A composition comprising a compound of formula (I), or a therapeutically acceptable salt or prodrug thereof and a therapeutically acceptable excipient.

6. A pharmaceutical composition according to claim 6, in the form of a tablet, capsule, powder, granules, syrup, solution or suspension.

7. A method for treating bacterial infections, psoriasis, arthritis in mammals comprising administering a therapeutically acceptable amount of compound of formula (I), or a therapeutically acceptable salt or prodrug thereof.

8. The method as claimed in claim 7 wherein the compound is administered orally, nasally, parenterally, topically, transdermally, or rectally.

9. A method for treating toxicity due to chemotherapy in a patient comprising administering a therapeutically acceptable amount of compound of formula (I), or a therapeutically acceptable salt or prodrug thereof.

10. The method as claimed in claim 9 wherein the compound is administered orally, nasally, parenterally, topically, transdermally, or rectally.

11. A compound according to claim 1 which is selected from:
(S)-N-[3-(3-Fluoro-4-{4-[3-(4-hydroxyphenyl)-acryloyl]-piperazin-1-yl]-phenyl)-2-oxo-oxazolidin-5-yl methyl]acetamide;
(S)-N-[3-(3-Fluoro-4-{4-[3-(4-hydroxyphenyl)-acryloyl]-piperazin-1-yl]-phenyl)-2-oxo-oxazolidin-5-yl methyl]thioacetamide;
(S)-N-[3-(3-Fluoro-4-{4-[3-(4-hydroxyphenyl)-acryloyl]-piperazin-1-yl]-phenyl)-2-oxo-oxazolidin-5-yl methyl]thiourea;
(S)-N-[3-(3-Fluoro-4-{4-[3-(3-hydroxyphenyl)-acryloyl]-piperazin-1-yl]-phenyl)-2-oxo-oxazolidin-5-yl methyl]acetamide;
(S)-N-[3-(3-Fluoro-4-{4-[3-(3-hydroxyphenyl)-acryloyl]-piperazin-1-yl]-phenyl)-2-oxo-oxazolidin-5-yl methyl]thioacetamide;
(S)-N-[3-{4-(4-(3-Benzo[1,3]-dioxol-5-yl-acryloyl)-piperazin-1-yl}-3-fluorophenyl]-2-oxo-oxazolidin-5-yl methyl]acetamide;
(S)-N-[3-{4-(4-(3-Benzo[1,3]-dioxol-5-yl-acryloyl)-piperazin-1-yl}-3-fluorophenyl]-2-oxo-oxazolidin-5-yl methyl]thioacetamide;
(S)-N-[3-{4-(4-(3-Benzo[1,3]-dioxol-5-yl-acryloyl)-piperazin-1-yl}-3-fluorophenyl]-2-oxo-oxazolidin-5-yl methyl]thiourea;
(S)-N-[3-(3-Fluoro-4-{4-[3-(thiophen-3-yl)-acryloyl]-piperazinyl]-phenyl)-2-oxo-oxazolidin-5-yl methyl]acetamide;
(S)-N-[3-(3-Fluoro-4-{4-[3-(thiophen-2-yl)-acryloyl]-piperazinyl]-phenyl)-2-oxo-oxazolidin-5-yl methyl]acetamide;
(S)-N-[3-(3-Fluoro-4-{4-[3-(thiophen-2-yl)-acryloyl]-piperazinyl]-phenyl)-2-oxo-oxazolidin-5-yl methyl]thioacetamide;
(S)-N-[3-(3-Fluoro-4-{4-[3-(thiophen-2-yl)-acryloyl]-piperazinyl]-phenyl)-2-oxo-oxazolidin-5-yl methyl]thiourea;
(S)-N-[3-(3-Fluoro-4-{4-[3-(thiophen-2-yl)-acryloyl]-piperazinyl]-phenyl)-2-oxo-oxazolidin-5-yl methyl]thiocarbamate;
(S)-N-[3-(3-Fluoro-4-{4-[3-(1H-indol-3-yl)-acryloyl]-piperazin-1-yl]-phenyl)-2-oxo-oxazolidin-5-yl methyl]acetamide;
(S)-N-[3-(3-Fluoro-4-{4-[3-(1H-indol-3-yl)-acryloyl]-piperazin-1-yl]-phenyl)-2-oxo-oxazolidin-5-yl methyl]thioacetamide;
(S)-N-[3-(3-Fluoro-4-{4-[3-(1H-indol-3-yl)-acryloyl]-piperazin-1-yl]-phenyl)-2-oxo-oxazolidin-5-yl methyl]thiourea;
(S)-N-[3-(3-Fluoro-4-{4-[3-(furan-2-yl)-acryloyl]-piperazin-1-yl]-phenyl)-2-oxo-oxazolidin-5-yl methyl]acetamide;
(S)-N-[3-(3-Fluoro-4-{4-[3-(furan-2-yl)-acryloyl]-piperazin-1-yl]-phenyl)-2-oxo-oxazolidin-5-yl methyl]thioacetamide;
(S)-N-[3-(3-Fluoro-4-{4-[3-(furan-2-yl)-acryloyl]-piperazin-1-yl]-phenyl)-2-oxo-oxazolidin-5-yl methyl]thiourea;
(S)-N-[3-(3-Fluoro-4-{4-[3-(pyridin-3-yl)-acryloyl]-piperazin-1-yl]-phenyl)-2-oxo-oxazolidin-5-yl methyl]acetamide;
(S)-N-[3-(3-Fluoro-4-{4-[3-(pyridin-3-yl)-acryloyl]-piperazin-1-yl]-phenyl)-2-oxo-oxazolidin-5-yl methyl]thioacetamide;
(S)-N-[3-(3-Fluoro-4-{4-[3-(pyridin-4-yl)-acryloyl]-piperazin-1-yl]-phenyl)-2-oxo-oxazolidin-5-yl methyl]acetamide;
(S)-N-[3-(3-Fluoro-4-{4-[3-(pyridin-4-yl)-acryloyl]-piperazin-1-yl]-phenyl)-2-oxo-oxazolidin-5-yl methyl]thioacetamide;
(S)-N-[3-(3-Fluoro-4-{4-[3-(pyridin-4-yl)-acryloyl]-piperazin-1-yl]-phenyl)-2-oxo-oxazolidin-5-yl methyl]thiourea;
(S)-N-[3-(3-Fluoro-4-{4-[3-phenyl-propanoyl]-piperazin-1-yl]-phenyl)-2-oxo-oxazolidin-1 5-yl methyl]acetamide;
(S)-N-[3-(3-Fluoro-4-{4-[3-phenyl-propanoyl]-piperazin-1-yl]-phenyl)-2-oxo-oxazolidin-5-yl methyl]thioacetamide;
(S)-N-[3-(3-Fluoro-4-{4-[3-(4-fluorophenyl)-acryloyl]-piperazin-1-yl]-phenyl)-2-oxo-oxazolidin-5-yl methyl]acetamide;
(S)-N-[3-(3-Fluoro-4-{4-[3-(4-fluorophenyl)-acryloyl]-piperazin-1-yl]-phenyl)-2-oxo-oxazolidin-5-yl methyl]thioacetamide;
(S)-N-[3-(3-Fluoro-4-{4-[3-(4-fluorophenyl)-acryloyl]-piperazin-1-yl]-phenyl)-2-oxo-oxazolidin-5-yl methyl]thiourea;
(S)-N-[3-(3-Fluoro-4-{4-[3-(4-fluorophenyl)-acryloyl]-piperazin-1-yl]-phenyl)-2-oxo-oxazolidin-5-yl methyl]thiocarbamate;
(S)-N-[3-(3-Fluoro-4-{4-[3-phenyl acryloyl]-piperazin-1-yl]-phenyl)-2-oxo-oxazolidin-5-yl methyl]acetamide;
(S)-N-[3-(3-Fluoro-4-{4-[3-phenyl acryloyl]-piperazin-1-yl]-phenyl)-2-oxo-oxazolidin-5-yl methyl]thioacetamide;
(S)-N-[3-(3-Fluoro-4-{4-[3-phenyl acryloyl]-piperazin-1-yl]-phenyl)-2-oxo-oxazolidin-5-yl methyl]thiourea;
(S)-N-[3-(3-Fluoro-4-{4-[3-(4-methoxyphenyl)acryloyl)-piperazin-1-yl]-phenyl)-2-oxo-oxazolidin-5-yl methyl]acetamide;
(S)-N-[3-(3-Fluoro-4-{4-[3-(4-methoxyphenyl)acryloyl)-piperazin-1-yl]-phenyl)-2-oxo-oxazolidin-5-yl methyl]thioacetamide;
(S)-N-[3-(3-Fluoro-4-{4-[3-(4-methoxyphenyl)acryloyl)-piperazin-1-yl]-phenyl)-2-oxo-oxazolidin-5-yl methyl]thiourea;
(S)-N-[3-(3-Fluoro-4-{4-[3-(4-acetoxyphenyl)acryloyl)-piperazin-1-yl]-phenyl)-2-oxo-oxazolidin-5-yl methyl]acetamide;
(S)-N-[3-(3-Fluoro-4-{4-[3-(4-acetoxyphenyl)acryloyl)-piperazin-1-yl]-phenyl)-2-oxo-oxazolidin-5-yl methyl]thioacetamide;
(S)-N-[3-(3-Fluoro-4-{4-[3-(4-acetoxyphenyl)acryloyl)-piperazin-1-yl]-phenyl)-2-oxo-oxazolidin-5-yl methyl]thiourea;
(S)-N-[3-(3-Fluoro-4-{4-[3-furan-3-yl-acryloyl)-piperazin-1-yl]-phenyl)-2-oxo-oxazolidin-5-yl methyl]acetamide;
(S)-N-[3-(3-Fluoro-4-{4-[3-(3,4-difluorophenyl)-acryloyl)-piperazin-1-yl]-phenyl)-2-oxo-oxazolidin-5-yl methyl]acetamide;
(S)-N-[3-(3-Fluoro-4-{4-[3-(3,4-difluorophenyl)-acryloyl)-piperazin-1-yl]-phenyl)-2-oxo-oxazolidin-5-yl methyl]thioacetamide;
(S)-N-[3-(3-Fluoro-4-{4-[3-(3,4-difluorophenyl)-acryloyl)-piperazin-1-yl]-phenyl)-2-oxo-oxazolidin-5-yl methyl]thioacetamide;
Methanesulfonic acid 4-[3-(4-{4-[5-(acetyl aminomethyl)-2-oxo-oxazolidin-3-yl]-2-fluorophenyl}piperazin-1-yl]-3-oxo-propenyl]-phenyl ester,
(S)-N-[3-(3-Fluoro-4-{4-[3-(4-methylsulfanyl-phenyl)-acryloyl)-piperazin-1-yl]-phenyl)-2-oxo-oxazolidin-5-yl methyl]acetamide;
(S)-N-[3-(4-{4-[3-(3,4-dihydroxyphenyl)-acryloyl)-piperazin-1-yl]-3-fluorophenyl)-2-oxo-oxazolidin-5-yl methyl]acetamide;
(S)-N-[3-(4-{4-[3-biphenyl-4-yl-acryloyl)-piperazin-1-yl]-3-fluorophenyl)-2-oxo-oxazolidin-5-yl methyl]acetamide;
(S)-N-[3-(4-{4-but-2-enoyl-piperazin-1-yl]-3-fluorophenyl)-2-oxo-oxazolidin-5-yl methyl]acetamide;
(S)-N-[3-(4-{4-acryloyl-piperazin-1-yl]-3-fluorophenyl)-2-oxo-oxazolidin-5-yl methyl]acetamide;
(S)-N-[3-(3-Fluoro-4-{4-[2-methylacryloyl-piperazin-1-yl]-phenyl)-2-oxo-oxazolidin-5-yl methyl]acetamide;
(S)-N-[3-(-4-{4-[3-(4-benzyloxy-phenyl)-acryloyl)-piperazin-1-yl]-3-fluorophenyl)-2-oxo-oxazolidin-5-yl methyl]thiourea;
(S)-N-[3-(4-{4-[3-(4-nitrophenyl)-acryloyl)-piperazin-1-yl]-3-fluorophenyl)-2-oxo-oxazolidin-5-yl methyl]acetamide;
Carbonic acid-1-{4-[3-(4-{4-[5-(acetylamino-methyl)-2-oxo-oxazolidin-3-yl]-2-fluorophenyl}-piperazin-1-yl)-3-oxo-propenyl]-phenoxy}-ethyl ether cyclohexyl ester;
(S)-N-[3-(4-{4-[3-(4-aminophenyl)-acryloyl)-piperazin-1-yl]-3-fluorophenyl)-2-oxo-oxazolidin-5-yl methyl]acetamide;
(S)-N-[3-(4-{4-[3-(3,4-diacetoxy-phenyl)-acryloyl)-piperazin-1-yl]-3-fluorophenyl)-2-oxo-oxazolidin-5-yl methyl]acetamide;
(S)-N-[3-(4-{4-[3-benzo[1,3]-dioxol-5-yl acryloyl)-piperazin-1-yl]-3-fluorophenyl)-2-oxo-oxazolidin-5-yl methyl]thiocarbamate;
(S)-N-[3-(3-Fluoro-4-[4-(4-oxo-4-phenyl-but-2-enoyl)-piperazin-1-yl]-phenyl)-2-oxo-oxazolidin-5-yl methyl]acetamide;
(S)-N-[3-(3-Fluoro-4-[4-(4-(4-methoxyphenyl)-4-oxo-but-2-enoyl)-piperazin-1-yl]-phenyl)-2-oxo-oxazolidin-5-yl methyl]acetamide;
(S)-N-[3-(3-Fluoro-4-[4-(4-(4-methoxyphenyl)-4-oxo-but-2-enoyl)-piperazin-1-yl]-phenyl)-2-oxo-oxazolidin-5-yl methyl]thioacetamide;
(S)-N-[3-{4-[4-(4-(4-acetylaminophenyl)-4-oxo-but-2-enoyl)-piperazin-1-yl]-3-fluorophenyl)-2-oxo-oxazolidin-5-yl methyl]acetamide;
(S)-N-[3-(3-Fluoro-4-[4-(4-(4-acetylaminophenyl)-acryloyl)-piperazin-1-yl]-3-fluorophenyl)-2-oxo-oxazolidin-5-yl methyl]acetamide;
(S)-N-[3-(3-Fluoro-4-[4-(3-cyclohexyl)-acryloyl-piperazin-1-yl]-3-fluorophenyl)-2-oxo-oxazolidin-5-yl methyl]acetamide;
Acetic acid-2-(4-{4-[5-(acetylaminomethyl)-2-oxo-oxazolidin-3-yl]-2-fluorophenyl][-piperazinyl-1-carbonyl-7-amino-3-oxo-5-thia-1-aza-bicyclo-[4.2.0]-oct-2-en-3-yl-methyl ester;
2,2-Dimethyl-propanoic acid-4-(3-(4-{4-[5-(acetylaminomethyl)-2-oxo-oxazolidin-3-yl]-2-fluorophenyl}piperazinyl-1-yl)-3-oxo-propenyl]phenyl ester;
Carbonic acid-1-{4-[3-(4-{4-[5-(acetylaminomethyl)-2-oxo-oxazolidin-3-yl]-2-fluorophenyl][-piperazinyl-1-yl)-3-oxo-propenyl]phenyl ester;
(S)-N-[3-(3-Fluoro-4-[4-(3-(5-nitrofuran-2-yl)-acryloyl-piperazin-1-yl]-3-phenyl)-2-oxo-oxazolidin-5-yl methyl]acetamide;
(S)-N-[3-(3-Fluoro-4-[4-(6-methoxy-1-oxo-1,2,3,4 tetrahydronaphthalen-2-yl methyl)-piperazin-1-yl]-phenyl)-2-oxo-oxazolidin-5-yl methyl]acetamide;
(S)-N-[3-(3-Fluoro-4-[4-(1-oxo-1,2,3,4 tetrahydronaphthalen-2-yl methyl)-piperazin-1-yl]-3-phenyl)-2-oxo-oxazolidin-5-yl methyl]acetamide;
(S)-N-[3-(3-Fluoro-4-[4-(5-methoxy-1-oxo-indan-2-yl-methyl)-piperazin-1-yl]-3-phenyl)-2-oxo-oxazolidin-5-yl methyl]acetamide;
(S)-N-[3-(3-Fluoro-4-[4-(2-oxo-cyclohexylmethyl)-piperazin-1-yl]-3-phenyl)-2-oxo-oxazolidin-5-yl methyl]acetamide;
(S)-N-[3-(3-Fluoro-4-[4-(6-methoxy-1-oxo-1,2,3,4 tetrahydronaphthalen-2-yl methyl)-piperazin-1-yl]-3-phenyl)-2-oxo-oxazolidin-5-yl methyl]thioacetamide;
(S)-N-[3-(3-Fluoro-4-[4-(5-methoxy-1-oxo-indan-2-yl-methyl)-piperazin-1-yl]-3-phenyl)-2-oxo-oxazolidin-5-yl methyl]thioacetamide;
(S)-N-[3-(3-Fluoro-4-[4-(1-hydroxyimino-6-methoxy-1,2,3,4 tetrahydronaphthalen-1-yl methyl)-piperazin-1-yl]-phenyl)-2-oxo-oxazolidin-5-yl methyl]acetamide;
(S)-N-[3-(3-Fluoro-4-[4-(4-methyl-1-oxo-1,2,3,4 tetrahydronaphthalen-2-yl methyl)-piperazin-1-yl]-phenyl)-2-oxo-oxazolidin-5-yl methyl]thioacetamide;
Trans-(S)-N-(3-{3-Fluoro-4-[4-(3-1H-pyrrol-2-yl-acryloyl)-piperazin-1-yl]-phenyl}-2-oxo-oxazolidin-5-yl-methyl)acetamide.
Cis-(S)-N-(3-{3-Fluoro-4-[4-(3-1H-pyrrol-2-yl-acryloyl)-piperazin-1-yl]-phenyl}-2-oxo-oxazolidin-5-yl-methyl)acetamide.
(S)-5-[3-(4-{4-[5-(Acetylamino-methyl)-2-oxo-oxazolin-3-yl]-2-fluoro-phenyl}-piperazin-1-yl)-3-oxo-propenyl]-furan-2-carboxlic acid sodium salt
(S)-5-[3-(4-{4-[5-(Acetylamino-methyl)-2-oxo-oxazolin-3-yl]-2-fluoro-phenyl}-piperazin-1-yl)-3-oxo-propenyl]-furan-2-carboxlic acid.
(S)-N-[3-(3-Fluoro-4-{4-[3-(5-hydroxymethyl-furan-2-yl)-acryloyl]-piperazin-1-yl}-phenyl)-2-oxo-oxazolidin-5-yl methyl]acetamide.
(S)-N-[3-(3-Fluoro-4-{4-[3-(4-methanesulfonyl-phenyl)-acryloyl]-piperazin-1-yl}-phenyl)-2-oxo-oxazolidin-5-yl methyl]acetamide.
(S)-4-(4-{4-[5-(Aceylamino-methyl)-2-oxo-oxazolidin-3-yl]-2-fluoro-phenyl}-piperazin-1-yl)-4-oxo-but-2-enoic acid.
(S)-N-[3-(3-Fluoro-4-{4-[3-(5-formyl-furan-2-yl)-acryloyl]-piperazin-1-yl}-phenyl)-2-oxo-oxazolidin-5-yl methyl]acetamide.
(S)-Acetic acid-5-[3-(4-{4-[5-(Acetylamino-methyl)-2-oxo-oxazolin-3-yl]-2-fluoro-phenyl}-piperazin-1-yl)-3-oxo-propenyl]-furan-2-yl methyl ester.
(S)-4-(4-{4-[5-(Aceylamino-methyl)-2-oxo-oxazolidin-3-yl]-2-fluoro-phenyl}-piperazin-1-yl)-4-oxo-but-2-enoic acid sodium salt.
(S)-N-[3-(3-Fluoro-4-{4-[3-(5-methyl-furan-2-yl)-acryloyl]-piperazin-1-yl}-phenyl)-2-oxo-oxazolidin-5-yl methyl]acetamide.
(S)-N-[3-(3-Fluoro-4-{4-propynoyl-piperazin-1-yl]-phenyl)-2-oxo-oxazolidin-5-yl methyl]acetamide;
(S)-N-[3-(3-Fluoro-4-{4-(4-hydroxy-but-2-enoyl)-piperazin-1-yl]-phenyl)-2-oxo-oxazolidin-5-yl methyl]acetamide;
(S)-N-[3-(3-Fluoro-4-{4-(4-bromo-but-2-enoyl)-piperazin-1-yl]-phenyl)-2-oxo-oxazolidin-5-yl methyl]acetamide;
2-[4-(4-{5-(acetylamino-methyl)-2-oxo-oxazolidin-3-yl}-2-fluorophenyl)-piperazin-1-carbonyl]-3-phenyl-acrylic acid methyl ester;
2-[4-(4-{5-(acetylamino-methyl)-2-oxo-oxazolidin-3-yl}-2-fluorophenyl)-piperazin-1-carbonyl]-3-phenyl-acrylic acid;
2-[4-(4-{5-(acetylamino-methyl)-2-oxo-oxazolidin-3-yl}-2-fluorophenyl)-piperazin-1-carbonyl]-3-furane acrylic acid methyl ester;
2-[4-(4-{5-(acetylamino-methyl)-2-oxo-oxazolidin-3-yl}-2-fluorophenyl)-piperazin-1-carbonyl]-3-furane-acrylic acid;

12. A pharmaceutical composition, which comprises a compound as defined in claim 11, and a pharmaceutically acceptable carrier, diluents or excipients or solvate.

13. A pharmaceutical composition as claimed in claim 12, in the form of a tablet, capsule, powder, granules, syrup, solution or suspension.

14. A method for treating bacterial infections, psoriasis or arthritis in mammals comprising administering a therapeutically acceptable amount of compounds of claim 11, or a therapeutically acceptable salt or prodrug thereof.

15. The method as claimed in claim 14 wherein the compound is administered orally, nasally, parenterally, topically, transdermally, or rectally.

16. A method for treating toxicity due to chemotherapy in a patient comprising administering a therapeutically acceptable amount of compounds of claim 11, or a therapeutically acceptable salt or prodrug thereof.

17. The method as claimed in claim 16 wherein the compound is administered orally, nasally, parenterally, topically, transdermally, or rectally.

18. A medicine for treating bacterial infections, psoriasis, arthritis in mammals comprising administering a therapeutically acceptable amount of compounds described in any preceding claims, or a therapeutically acceptable salt or prodrug thereof.

19. A medicine for treating toxicity due to chemotherapy in a patient comprising administering a therapeutically acceptable amount of compound described in any preceding claims, or a therapeutically acceptable salt or prodrug thereof.

20. The medicine as claimed in any preceding claims wherein the compound is administered orally, nasally, parenterally, topically, transdermally, or rectally.

21. A process for the preparation of a compound of formula (I) as claimed in claim 1, where all symbols are as defined earlier, and including their derivatives, their analogs, their tautomeric forms, their stereoisomers, their pharmaceutically acceptable salts, their pharmaceutically acceptable solvates, which comprises:
i. by reacting a compound of formula (1a) with a compound of formula (1b) 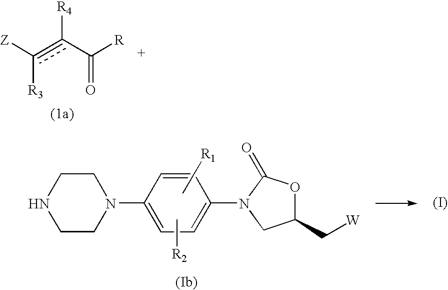
where all symbols are as defined earlier and R represents OH, halide or an acyloxy group, to yield compound of formula (I).
ii) by reacting a compound of formula (1c) with a compound of formula (1b) 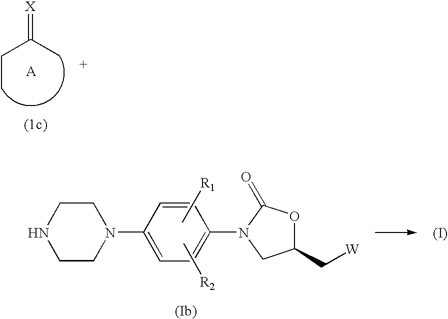
where all symbols are as defined earlier, to yield compounds of formula (I).
iii) Reacting a compound of formula (1m) with a compound of formula (1b) to give compound of formula (1n): 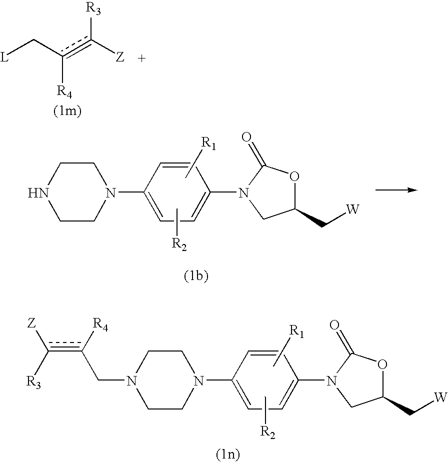
where all symbols are as defined earlier; The compound (In) represents compound of formula (1), where Y represents G₃ as defined in claim 1.

22. A process of converting compounds of formula (I) to further compounds of formula (1), which comprises:
a) reacting of a compound of formula (1a) with a compound of formula (1d) to yield (1e), 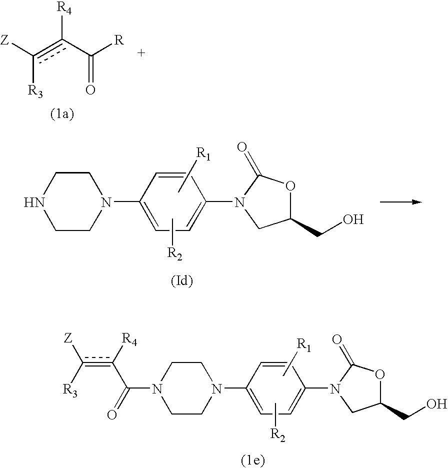
b) Converting a compound of formula (1e) to (1f) where L represents a leaving group such as —OMs, —OTs, halides etc. 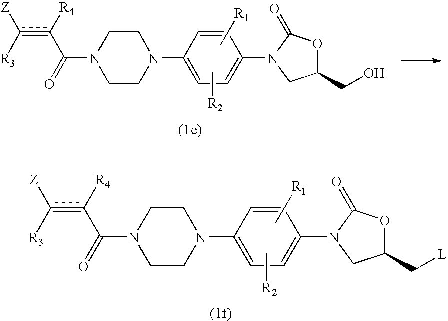
c) Converting compound (1f) to (1g) 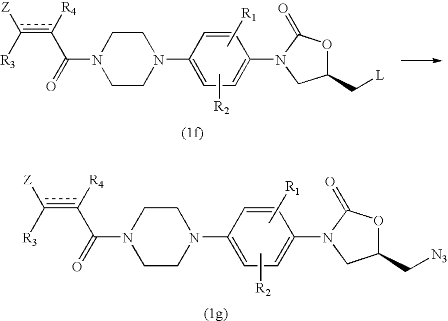
d) Converting compound (1g) to (1h) 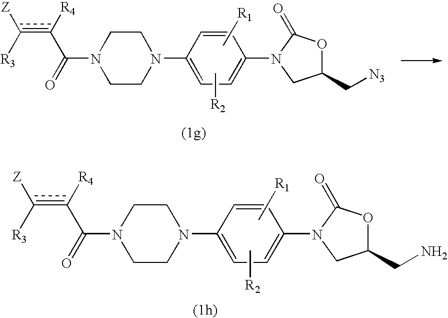
e) Converting (1h) to (1i) 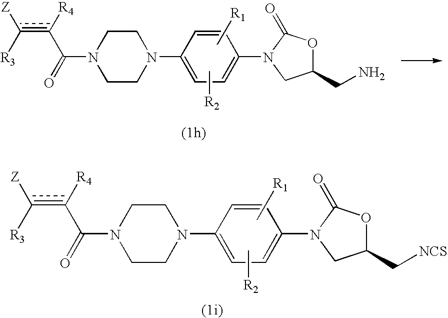
f) Converting (1i) to (1j) 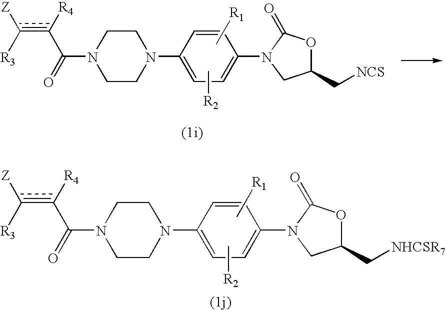
Alternatively,
g) Converting compound (1i) to (1k) 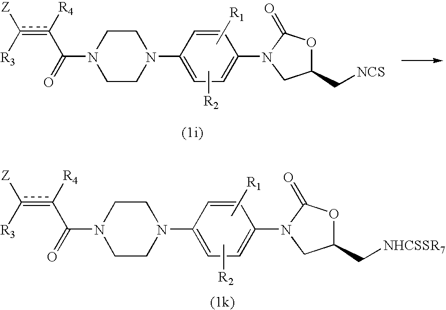
Alternatively
h) Converting compound (1i) to (1l) 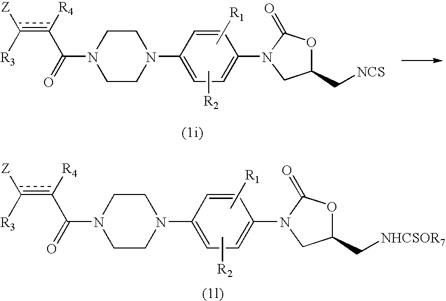
where all symbols are as defined earlier and compounds of formula (Ie), (Ig), (Ih), (Ii), (Ij), (Ik), (Il), represent compounds of formula (I), and W represents OH, N₃, NH₂, NCS, NHCSR₇, NHCSSR₇, NHCSOR₇ respectively, and Y represents G₂ with X=O.
